(19) United States
(12) Patent Application Publication
(10) Pub. No.: US 2024/0369540 A1
(43) Pub. Date: Nov. 7, 2024

(54) MECHANISMS AND PREDICTORS OF ADJUVANTICITY AND ANTIBODY DURABILITY

(71) Applicant: The Board of Trustees of the Leland Stanford Junior University, Stanford, CA (US)

(72) Inventors: Bali Pulendran, Redwood City, CA (US); Thomas Hagan, Redwood City, CA (US); Mario Cortese, Redwood City, CA (US); Sheng-Yang Wu, Redwood City, CA (US)

(21) Appl. No.: 18/567,325

(22) PCT Filed: Jun. 14, 2022
(86) PCT No.: PCT/US2022/033428

§ 371 Date: Dec. 5, 2023

Related U.S. Application Data
(60) Provisional application No. 63/210,794, filed on Jun. 15, 2021.

(51) Int. Cl. G01N 33/50

(52) U.S. Cl. CPC G01N 33/5088; G01N 33/5023; G01N 2500/00

(57) ABSTRACT

Methods are provided herein for vaccine development, characterization and validation. Using the response signatures disclosed herein, methods are provided for optimization, selection and benchmarking of vaccines, including adjuvants for vaccines. The methods include a prediction of response durability. e.g. the longevity of an antibody response, for a candidate vaccine or vaccine adjuvant; and assessment of similarity to a benchmark reference vaccine.

### CROSS REFERENCE TO RELATED APPLICATION

The present application claims the benefit of and priority to U.S. Provisional Patent Application No. 63/210,794, filed Jun. 15, 2021, the entire disclosure of which is hereby incorporated by reference in its entirety.

### STATEMENT REGARDING FEDERALLY SPONSORED RESEARCH

This invention was made with Government support under contract AI090023 awarded by the National Institutes of Health. The Government has certain rights in the invention.

### BACKGROUND

With over 80 million cases and 1.7 million deaths around the world as of December 2020, COVID-19 has demonstrated the grave threat that global pandemics represent for public health. The ability to quickly develop vaccines that induce protective immunity against novel pathogens is crucial in controlling and preventing such pandemics. As vaccine components that enhance the magnitude, breadth, and durability of the immune response, adjuvants are powerful tools for modern vaccine development. By enabling antigen-sparing, adjuvants also allow for more rapid vaccine production, a critical factor during response to pandemics. For more than 70 years, insoluble aluminum salts (alum) were the only licensed adjuvant, however in the past 3 decades there has been a large expansion in adjuvants available in licensed vaccines. These include oil-in-water emulsion-based adjuvants (MF59, AS03), adjuvants containing the TLR4 agonist 3-O-desacyl-4′-monophosphoryl lipid A (MPL) (AS01, AS04), and CpG 1018, a TLR9 agonist CpG oligonucleotide. Many of these adjuvants, including AS03, MF59, and CpG 1018, have been made available by their owners for use in COVID-19 vaccines, and at least 10 developers have indicated plans to create adjuvanted COVID-19 vaccines. Despite this large growth in adjuvant technology, in many cases the molecular mechanisms by which these adjuvants boost immune responses to vaccination remain unclear.

AS03 is a squalene-based oil-in-water emulsion containing α-tocopherol (Vitamin E), and has been shown to increase the breadth and magnitude of CD4+ T cell and antibody responses against multiple strains of influenza, even compared with MF59. Recent work in mice demonstrated that AS03 induced alterations in expression of lipid metabolism-related genes in the draining lymph nodes, as well as increased endoplasmic reticulum (ER) stress in macrophages, which drove elevated cytokine production and improved antibody responses. Additionally, the similar oil-in-water emulsion-based adjuvant MF59 has been shown to induce a local release of extracellular ATP, and to depend functionally on MyD88 in an inflammasome-independent fashion, suggesting that these type of adjuvants produce some degree of cell injury or stress that result in release of damage-associated molecular patterns (DAMPs) and induction of innate immune responses. However, the molecular pathways through which AS03 promotes these responses remain poorly defined.

In addition to boosting the initial immune response to vaccination, adjuvants, including AS03 and others, can also improve the longevity of the resulting immunity. Whereas some vaccines, particularly live viral vaccines such as smallpox or yellow fever, can induce lifelong antibody responses, others, such as those against pertussis and influenza, only promote transient responses and immunity that wanes over time, resulting in a loss of protection and need for booster vaccinations. With regards to humoral immunity, long-lived plasma cells have been identified as key mediators of durable antibody responses, but the mechanisms required to drive robust long-lived plasma cell differentiation and persistent antibody responses to vaccination are not well understood.

Although live attenuated vaccines such as smallpox or yellow fever vaccines induce durable antibody (Ab) responses that can last a lifetime, waning immunity has been documented with several vaccines, including the mRNA vaccines against COVID-19, and subunit vaccines against influenza, malaria, Bordetella pertussis, Salmonella enterica serovar Typhi, and Neisseria meningitidis and other pathogens. Why some vaccines provide lifetime protection and others protect for only a few months remains one of the great mysteries of immunology. Currently, the duration of immune protection for new vaccines is difficult to predict during vaccine product development and can only be ascertained by a “wait and see” approach. Therefore, a grand challenge for vaccinology is to be able to predict how long a vaccine will be protective before, by defining early signatures (gene signatures or cell based signatures) in the blood, induced within a few days of vaccination, that predict the durability of immune response and protection.

### SUMMARY

Methods are provided for vaccine development and validation. Using the genetic signatures disclosed herein, methods are provided for optimization of vaccines, including adjuvants for vaccines, and predicting the durability of antibody responses. The methods include a prediction of response durability, e.g. the longevity of an antibody response, for a candidate vaccine or vaccine adjuvant. Vaccines of interest include, for example, live virus vaccines. subunit vaccines, mRNA vaccines, viral vector vaccines, etc.

The methods provide a means of predicting durability of response in a short period of time following immunization, e.g. with less than about 14 days, less than about 10 days, e.g. up to or including at 7 days. This information allows a rapid benchmarking and stratification of vaccine effectiveness, providing a significant benefit of shortening the time required for evaluation.

In an embodiment, a method is provided for predicting the durability of an immune response to a candidate vaccine, the method comprising administering the candidate vaccine, which may comprise an adjuvant, to a mammal; determining an early gene signature from immune cells, for example peripheral blood mononuclear cells (PBMCs); and predicting durability of response from the early gene signature. In some embodiments the immune cells comprise platelets. In some embodiments the early gene signature is determined by mRNA content from platelets. In some embodiments the early gene signature is determine from about 7 to about 10 days following immunization. In some embodiments the mammal is a mouse. In some embodiments the mammal is a non-human primate. In some embodiments the mammal is a human. In some embodiments, an analysis of plasma metabolomics is performed on the mammal. In some embodiments, a candidate vaccine or adjuvant is selected for development based on the ability to provide an early gene signature indicate of greater antibody longevity.

In an embodiment, a method is provided for predicting the durability of an immune response to a candidate vaccine, the method comprising administering the candidate vaccine, which may comprise an adjuvant, to a mammal; and determining the RNA content of platelets in the recipient following vaccination. In some embodiments RNA content of platelets is determined from about 7 to about 10 days following immunization. In some embodiments the mammal is a mouse. In some embodiments the mammal is a non-human primate. In some embodiments the mammal is a human. In some embodiments, analysis of platelet RNA content is performed by flow cytometry. The fold change in platelet RNA content can be compared to the baseline level prior to vaccination, where a durable immune response is associated with an increase of at least about 5-fold, at least about 10-fold, at least about 20-fold relative to baseline. In some embodiments, platelets are defined as CD41⁺CD61⁺ cells after the exclusion of CD3⁺, CD8⁺, CD20⁺ and CD14⁺ cells. In some embodiments, a candidate vaccine or adjuvant is selected for development based on the ability to increase platelet RNA content indicative of greater antibody longevity.

In other embodiments, methods are provided for determining whether a candidate adjuvant provides for a core response induced specifically by a target high performing reference adjuvant, e.g. AS03. By using the day 1 changes in expression of three of these genes, TGM2, ANKRD22, and KREMEN1, adjuvant use was predicted. In some embodiments a method of selecting a candidate adjuvant with desirable properties is provided, the method comprising the method comprising administering a vaccine with the candidate adjuvant, to a mammal; determining an a core response signature from immune cells, for example peripheral blood mononuclear cells (PBMCs); and predicting whether the candidate adjuvant induces the core response by day 1 chages in expression. In some embodiments the mammal is a mouse. In some embodiments the mammal is a non-human primate. In some embodiments the mammal is a human. In some embodiments, a candidate adjuvant is selected for development based on the ability to provide a high performing core response at day 1 post vaccination.

An in-depth multi-omics analysis of cellular, transcriptional, and metabolic responses to a vaccine, with and without adjuvant, was performed, and a key set of genes induced specifically by adjuvant in immune cells was identified. Pathway analysis of these genes shows a role for apoptosis in the adjuvant mechanism of action; and plasma metabolomics analysis shoed the adjuvant-induced perturbations in lipid and fatty acid metabolism were highly associated with expression of the apoptotic signature. An early gene signature capable of successfully predicting antibody response longevity was derived in a cohort of vaccines. Subsequent single cell profiling revealed differences in RNA content among platelets as a major driver of this signature, which reflectes cell adhesion-related durability of antibody response.

Differentially expressed genes (DEGs) post-vaccination were determined, with the large majority of DEGs observed at day 1 post-prime and boost. To identify the specific pathways activated in response to vaccination, a gene set enrichment analysis (GSEA) was performed on genes ranked by post-vaccination fold change, using a set of blood transcriptional modules (BTMs). Merging BTM enrichment scores according to high-level functional categories revealed that adjuvant increased expression of a broad range of innate and adaptive immune cells and pathways on day 1 and 7 post-prime and boost vaccination, with strong enrichment of BTMs related to monocyte and dendritic cell (DC) activation at early time-points after each immunization, while day 7 responses were mostly dominated by robust B cell and plasma cell transcriptional responses.

To transcriptional signatures associated with a persistent antibody response, GSEA was performed on genes ranked by their correlation with the day 100/day 42 residual antibody titer. Expression of cell cycle-related modules on day 7 post-prime and cell adhesion/platelet activation-related modules on days 1-7 post-boost were associated with increased persistence. In particular, genes within the platelet activation/actin binding module showed strong agreement in their correlations with antibody persistence.

Transcriptional differences, both quantitative and qualitative, were observed in the innate immune responses following prime and boost immunization with adjuvant. A blood transcriptional signature of cellular migration associated with a more persistent antibody response to adjuvanted vaccination was identified and used to successfully predict antibody durability. CITE-seq analysis revealed that waning antibody responders showed a much sharper decrease in platelet RNA content after the second vaccination compared to more persistent responders.

By performing a meta-analysis of adjuvanted and unadjuvanted vaccine datasets, a common set of genes induced specifically by a target adjuvant was identified. By using the day 1 changes in expression of three of these genes, TGM2, ANKRD22, and KREMEN1, adjuvant use was predicted. Early transcriptional changes in the ‘core’ genes were strongly associated with frequencies of activated Tfh cells in the periphery 7 days after vaccination, showing participation for these genes in mechanisms of immunogenicity.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1. AS03 induces potent early transcriptional signatures which are enhanced after a booster vaccination (A) Study overview. A total of 50 healthy subjects aged 21-45 years old were randomized 2:1 to receive two doses 21 days apart of a monovalent, split-virion, inactivated H5N1 clade 2.1 A/Indonesia/05/2005 influenza vaccine, administered with (n=34) or without (n=16) the AS03 adjuvant. Biologic samples were collected and analyses performed at regular intervals (gray squares) as illustrated in the diagram. (B) Number of DEGs (p<0.01 and log 2 FC>0.2) post-vaccination in adjuvanted (orange) and nonadjuvanted (green) subjects. (C-D) Average blood transcriptional module (BTM) enrichment scores by cell type/pathway on day 1 (dark) and day 7 (light) after prime (C) and boost (D). (E) Enrichment scores of interferon-related BTMs on days 1-7 after prime (top) and boost (bottom). (F) Scatter plot of day 1 fold changes (x prime, y-boost), for BTMs differentially expressed (FDR<0.03) in adjuvanted subjects on day 1 between prime and boost. BTMs are color-coded as indicated in the legend. (G) Genes in BTM M111.1; each “edge” (gray line) represents a coexpression relationship (as described in Li et al., Nat. Immunol. 2014); colors represent the day 1 fold change after prime (left) and boost (right). (H) Scatter plot of day 3 fold changes (x-prime, y-boost), for BTMs differentially expressed (FDR<0.03) in adjuvanted subjects on day 3 between prime and boost. BTMs are color-coded as indicated in the legend. (I) Genes in BTM 89.0; each “edge” (gray line) represents a coexpression relationship; colors represent the day 3 fold change after prime (left) and boost (right).

FIG. 2. Adjuvanted H5N1 vaccination promotes protective H5-head directed antibody responses whose durability is associated with a transcriptional signature of cellular migration (A) Microneutralization (MN) titers against the H5N1 A/Indonesia vaccine strain in adjuvanted (orange) and non-adjuvanted (green) subjects. Geometric means are presented in thick lines, while shades are for geometric standard deviations (SD). (B) H5 head: stem IgG binding capacity ratio as measured by surface plasmon resonance (SPR). (C) Day 42 post-vaccination fold change in IgG affinity against the H5 head (left) and stem (right) as measured by SPR. (D) Heat map of BTMs whose post-vaccination activity is associated with antibody persistence in both H5N1+AS03 (orange) and TIV (pink) vaccine responses. Gene set enrichment analysis (GSEA, FDR<0.05; 1,000 permutations) (Subramaniam et al. 2004) was used to identify positive (red), negative (blue), or no (gray) enrichment of BTMs within pre-ranked gene lists, where genes were ranked according to their correlation with the residual of the day 100 versus day 42 (H5N1+AS03) or day 180 versus day 28 (TIV) antibody response. (E-F) Genes in BTM M196; each “edge” (gray line) represents a coexpression relationship (as described in Li et al., Nat. Immunol. 2014); colors represent the correlation of the day 7 gene expression with the day 100 (H5N1+AS03, E) or 180 (TIV, F) antibody response residual (positive-red, negative-blue) vaccination. (G) Scatterplot of actual versus predicted day 100 antibody response residuals in the CCHI dataset. Predicted day 100 antibody response residuals were generated using a linear regression based approach trained on day 7 transcriptional correlates of persistent antibody responses in the H5N1+AS03 and TIV datasets. See Methods section for further details. (H) Bar plot of genes from BTMs selected by the predictive model (G) whose expression on day 7 significantly correlates with antibody longevity (day 100 or day 180 response residual) in the H5N1+AS03 and TIV datasets. Bars represent the meta-correlation coefficient from both datasets. *p<0.05; **p<0.01; ***p<0.001; ****p<0.0001.

FIG. 3. CITEseq analysis reveals a platelet origin for transcriptional signature of antibody persistence (A) UMAP representation of PBMCs from all analyzed samples (n=12, 3 ‘persistent’ and 3 ‘waning’ responders, day 21 and day 28 samples from each subject) colored by manually annotated cell type. (B) UMAP representation of PBMCs from all analyzed samples showing the per-cell sum of expression for all genes in the predictive signature of antibody persistence (FIG. 2H). (C) Left panel: boxplot of pseudobulk expression of the antibody persistence signature among persistent and waning responders. Right panel: line graph of changes in pseudobulk expression of the antibody persistence signature when a given cell cluster is removed from the pseudobulk calculation. One-sided, unpaired t-test was used to compare groups. (D) Platelet fraction of total cells in persistent and waning responders on days 21 and 28 post first vaccination. (E) Fraction of total reads within the platelet cluster in persistent and waning responders on days 21 and 28 post first vaccination. (F) Bar chart of significantly enriched BTMs (FDR<0.05) via overrepresentation testing of DEGs (p≤0.05 & fc≥0.25) between persistent and waning responders within plasmablasts at day 28 after vaccination. (G) Heatmaps of genes within M238, M219, and M216 (left), M4.1 (center), and M250 (right) modules. Colors represent row-wise z scores of average expression within plasmablasts.

FIG. 4. Blood circulation of activated, vaccine-induced Tfh cells correlates with neutralizing antibody titers and antibody affinity maturation (A) Frequency of activated Tfh cells post-vaccination in adjuvanted (orange) and non-adjuvanted subjects (green), defined as percentage of PD1+ICOS+ cells within the CXCR5+ CD4+ T cell population. (B) Correlation of the day 28/21 fold change in activated Tfh cell frequencies with the day 42/21 fold increase in MN titers. (C) Correlation of the day 28/21 fold change in activated Tfh cell frequencies with the day 42/21 fold increase in IgG affinity against the H5 head. (D) Average log₂ fold change of genes in activated (PD-1+ICOS+) versus non-activated (PD-1-ICOS-) Tfh1 (x-axis) and Tfh2 (y-axis) cells. The top 20 genes with the greatest average absolute fold change are annotated in red. (E) Estimated frequency of monocytes in non-activated and activated Tfh based on digital cytometry of transcriptional profiles using CIBERSORT. (F) BTMs significantly enriched (FDR<0.05) in activated versus non-activated Tfh cells. CIBERSORTx was used to estimate CD4 T cell specific expression in sorted Tfh transcriptional profiles, and then GSEA (Subramaniam et al. 2004) was used to identify enriched BTMs using genes ranked by their fold change between activated and non-activated Tfh. See Methods section for further details. (G) Genes in BTM M219; each “edge” (gray line) represents a coexpression relationship (as described in Li et al., Nat. Immunol. 2014); colors represent the fold change in activated versus non-activated Tfh. (H) Genes in BTM M4.2; each “edge” (gray line) represents a coexpression relationship; colors represent the fold change in activated versus non-activated Tfh. (I) Clustered heatmap of the top 40 genes by fold change between activated and non-activated Tfh.

FIG. 5. An early molecular signature is associated with multiple markers of post-boost immune response (A) Schematic showing the identified associations between day 1 gene signatures, and increases in day 28 activated Tfh frequencies, day 42 lgG affinity against the H5 head, and day 42 microneutralization titers. (B) 3D scatter plot of the post-boost fold changes in activated Tfh frequencies (day 28), IgG affinity against the H5 head (day 42), and microneutralization titers (day 42) in adjuvanted (orange) and non-adjuvanted (green) subjects. (C) Barplot of BTMs commonly associated (FDR<0.05) with all 3 parameters (day 28 activated Tfh, day 42 H5 head IgG affinity, and day 42 MN titers). GSEA was performed on genes ranked by correlation with each parameter. (D) 3D scatter plot of day 1 BTM associations with post-boost fold changes of each immune parameter. The axes represent the GSEA enrichment scores of BTMs within pre-ranked gene lists, where genes were ranked according to their correlation with each immune parameter. (E) Heatmap of genes whose expression on day 1 significantly correlates (p<0.001) with all 3 immune parameters. Colors represent the day I/O fold change.

FIG. 6. Meta-analysis of influenza vaccine trials reveals an AS03-specific transcriptional signature which correlates with activated Tfh cell frequencies in the periphery (A) Identification of an AS03-specific gene signature. Data were incorporated from both the prime and boost doses of our trial, as well as publicly available data from a previous study of responses to AS03-adjuvanted H1N1 vaccination, and compared in a pairwise fashion with gene expression data from multiple TIV trials. See Methods section for further details. (B) Heatmap of genes differentially expressed (p<0.005 and log 2 FC>0.2) in all pairwise comparisons between AS03-adjuvanted and non-adjuvanted influenza vaccine trials. Of these, 3 ‘core’ genes (ANKRD22, KREMEN1, and TGM2) shared a large number of co-correlating genes. (C) Predicted adjuvant status of adjuvanted (orange) and non-adjuvanted (green) vaccine recipients in an independent trial. An artificial neural network-based machine learning classification algorithm was trained using day 1 expression data of ‘core’ AS03-specific genes within our study to predict vaccine status (adjuvanted vs. non-adjuvanted), and tested on an independent dataset containing expression data from a previously published study of responses to H5N1 vaccination with or without AS03. The dot plot displays the outcome of 10 randomized bootstrap trials, with each dot representing the ensemble vote of the classifier (1-adjuvanted, 0-non-adjuvanted) within a single trial. See Methods section for further details. (D) Classification accuracy of the ‘core’ AS03-gene classifier trained within our study and applied to two independent datasets. See Methods section for further details. (E) Correlation of day 22 expression of the 3 ‘core’ AS03-specific genes with the day 28/21 fold change in activated Tfh cell frequencies. (F) Correlation of day 1 expression of the TMEM159 gene with the day 42/0 fold change in MN titers. (G) Correlation of day 1 expression of the TMEM159 gene with the day 63/−7 fold change in MN titers in a publically available dataset where AS03 was co-administered with a monovalent H1N1 vaccine.

FIG. 7. Generation of early apoptotic signals after AS03 vaccination is associated with perturbation in fatty acid metabolism and oxidation (A-B) Differentially expressed genes (FDR<0.05) in apoptosis-related pathways (Reactome database) between the AS03+H5N1 group vs H5N1 and TIV datasets (pairwise comparisons) on day 1 (A) and day 22 (B) post vaccination. Genes belonging to specific apoptosis sub-pathways are color-coded in green (intrinsic pathway), magenta (extrinsic pathway), blue (execution phase), or orange (regulation). (C) Post-prime metabolic trajectories along the first two principal components for adjuvanted and non-adjuvanted subjects relative to day 0. Here metabolic trajectories refer to the trajectory of each subject according to the changes in abundance across all differential metabolite features (p<0.01) throughout the time course (days 1-7) when projected in the principal component space. (D) Post-boost metabolic trajectories along the first two principal components for adjuvanted and unadjuvanted subjects relative to day 21. (E) Metabolic pathways significantly enriched (p<0.05) in adjuvanted subjects on day 1 following vaccination. Circle size represents number of detected differentially abundant metabolites within the pathway. (F) Heatmap of metabolic pathways associated with apoptotic gene expression or post-boost immune responses on day 22. Mummichog software was used to identify enriched pathways based on metabolite features correlated (p<0.05 by Spearman correlation) with apoptotic gene expression or immune responses. Color and circle size represent the −log 10 p value of significant pathway enrichments (p<0.1) by permutation test. Pathways shown are those with an enrichment p value<0.01 with at least one feature.

FIG. 8. (A) Scatterplot of the mean log 2 FC of all BTMs in adjuvanted subjects on day 1 (x axis) and in nonadjuvanted subjects on day 3 (y axis). (B) Kinetics of differential BTMs between day 3 prime and boost. Lines represent average module fold change among adjuvanted subjects. The 10 BTMs with the greatest fold change on day 24 are plotted (same as those labeled in FIG. 1G). (C) Hemagglutination inhibition (HAI) titers against the H5N1 A/Indonesia vaccine strain in adjuvanted (orange) and non-adjuvanted (green) subjects. Geometric means are presented in thick lines, while shades are for geometric standard deviations (SD). (C) MN titers against heterologous clade 2 H5N1 strains. Geometric means are presented in thick lines, while error bars represent geometric standard deviations (SD). (D) H5 head and stem IgG binding capacity in resonance units (RU) as measured by surface plasmon resonance (SPR). Median values and interquartile ranges are shown in boxplots, violin plots show sample distributions. (F) Correlation of the day 42/21 fold change in IgG antibody binding against the H5 head and MN titers. (G) Correlation of the day 42/21 fold change in IgG affinity against the H5 head and MN titers.

FIG. 9. (A) Kinetics of HAI titers in response to H5N1+AS03 and TIV vaccination. Lines represent the geometric mean, and shaded areas represent the geometric standard deviation. TIV titers are from young adults (<65 years old) vaccinated with the 2010 and 2011 seasonal influenza vaccine (n=42) (Nakaya et al., 2015). (B) Correlation of the day 100 and day 42 HAI titers. (C) Heatmap of the mean log 2 FC of plasma cell and cell cycle BTMs in adjuvanted subjects. (D) Scatterplot of the day 28/day 21 mean log 2 FC of M156.0 (x axis) versus the day 100/day 42 HAI residual in adjuvanted subjects.

FIG. 10. (A) Per-cluster cell proportions for all analyzed cells before QC filtering. Left panel: proportion of cells in each cluster from day 21 and 28 samples. Right panel: proportion of cells in each cluster from each subject. (B) Scatterplots of day 28/21 FCs among day 28 DEGs via microarray (x axis) and pseudobulk estimates via CITEseq (y axis) for each subject. Statistics were generated using Pearson correlation. (C) Boxplots of day 28/21 FCs within platelets of antibody persistence signature genes for each subject using cell-wise normalized expression.

FIG. 11. (A) Per-cell QC metrics by cluster before QC filtering. (B) DEGs in each cluster compared to all other clusters before QC filtering. (C) CITE-seq antibody abundance in each cell before QC filtering.

FIG. 12. (A) Gating strategy for sorting of four different CD4+ CXCR5+ Tfh populations: quiescent Tfh1, quiescent Tfh2, activated Tfh1, and activated Tfh2. (B) NES of BTMs significantly enriched (FDR<0.05 and NES>2.5) in sorted activated versus unactivated Tfh cells prior to deconvolution using CIBERSORTx (Newman et al. 2019). GSEA (Subramaniam et al. 2004) was used to identify enriched BTMs using genes ranked by their average fold change between sorted activated and unactivated Tfh samples. (C) Estimated relative cellular fractions of various immune cells in sorted Tfh samples via CIBERSORT (Newman et al. 2015).

FIG. 13. (A) Heatmap of BTMs commonly enriched (FDR<0.001) in response to both TIV and H5N1+AS03 (prime or boost) on day 1 or 7 following vaccination. Color represents the NES, nonsignificant scores are shaded grey. (B) Difference in average NES between AS03 and TIV datasets of BTMs uniquely enriched in AS03 datasets. GSEA (cite Subramanian et al., 2005) was used to identify enrichment of BTMs using ranked gene lists, where genes were ordered by t-statistic based on day 1 versus day 0 fold change in AS03 and TIV datasets (see FIG. 5A and methods). BTMs shown are those significantly enriched (FDR<0.05) in all AS03 datasets but in no TIV datasets. (C) Genes in BTM M23; each “edge” (gray line) represents a coexpression relationship; colors represent the average day 1 fold change in all AS03 or TIV studies (see FIG. 5A and methods). (D) Overlap coefficient of significantly correlating partner genes (FDR<0.1) on day 1 between all ‘AS03-specific’ genes. (E) Estimated gene expression of ‘AS03-specific’ genes in different cell types on day 1 for the adjuvanted group.

FIG. 14A-M. A positive correlation between the fold change in platelet RNA content at day 7 versus day 0 post last immunization and the persistence of antibody response in the studies of humans, Rhesus macaques and mice. (A) Experimental timeline of platelet staining and HAI assay in seasonal influenza vaccination. Subjects were vaccinated with one dose of TIV (Fluzone®, Sanofi Pasteur Inc., in 2010-11 season) (B) Scatterplots of the day 180/day 28 HAI residual versus the day 7/day 0 log 2 fold change of RNA content (median RNA dye intensity) in whole platelet and % RNA⁺ platelets in vaccinated subjects. (C) Experimental timeline of platelet staining and neutralizing assay in Rhesus macaques immunized with RBD or hexamer of SARS-CoV-2 Spike immunogen admixed with AS03. (D) Line graph of the kinetics of SARS-COV2019 virus neutralizing antibody titers in serum. (E) Scatterplots of the day 180/day 42 nAb residual versus the day 28 (d7)/Baseline (d0) log 2 FC of RNA content in whole platelet and % RNA⁺ platelets in adjuvanted subjects. (F) Experimental timeline of platelet staining, neutralizing assay, and EPISPOT in Rhesus macaques immunized with the HIV-1 clade C-derived 1086.C gp140 immunogen mixed with R848 or MPL (TLR-4-targeted monophosphoryl lipid A)+R848. (G) Line graph of the kinetics of tier 1A MW965.26 HIV-1 pseudo-virus neutralizing antibody titers in serum. (H, I) Scatterplots of the week 19 (d7)/Baseline (d0) log 2 FC of RNA content in whole platelet versus the week 42/week 20 nAb residual and bone marrow ASC numbers in adjuvanted subjects. (J) Experimental timeline of platelet staining, ELISA, and ELISPOT in C57BL/6J mice immunized with SARS-COV-2 (2019-nCOV) Spike immunogen admixed with AS03. (K) Line graph of the kinetics of anti-Spike binding antibody titers in serum. (L, M) Scatterplots of the day 7/day 0 log 2 FC of RNA content in whole platelets versus the day 42/day 7 residual and the bone marrow ASC numbers.

FIG. 15. (A, D, F, H) The gating strategy for cell-free platelets in thawed PBMCs of human subjects in 2010 seasonal influenza vaccination (A), Rhesus macaques immunized with SARS-COV-2 Spike immunogen admixed with AS03 (D), Rhesus macaques immunized with HIV gp140 immunogen admixed with R848 or MPL+R848 (F), or cell-free platelets in freshly-prepared platelet-enrich plasma of B6 mice immunized with 2019-n-COV Spike immunogen admixed with AS03 (H). (B) The % platelets at days 0 and 7, and the fold change in the % platelets at day 7 versus day 0 in the gated population. (C, E, G, I) The fold changes in RNA content (median RNA dye intensity) in whole platelets or % RNA⁺ platelets in vaccinated subjects at day 7 post-last immunization versus on day of last immunization (C, I) or baseline (E, G).

Table 1. Demographics information for the 50 subjects enrolled in the two arms of the study.

Table 2. Geometric mean titers (GMT), 95% confidence intervals (CI), and seroconversion rates for both HAI and MN titers for the unadjuvanted and AS03-adjuvanted groups. Seroconversion rate is defined as the percentage of vaccines with a 4-fold or greater post-vaccination increase in titer over baseline levels.

### DETAILED DESCRIPTION

Compositions and methods are provided for classification of vaccines, including particularly vaccine adjuvants, for durability of quality of response, based on changes in gene expression at early time points following vaccination. Patterns of response are obtained by quantitating signals in immune cell subsets of interest, after a period of time, e.g. from 1 to 10 days post-vaccination, including day 7 post-vaccination. The pattern of response is indicative of the propensity have a response benchmarked to a reference adjuvant; and for longevity of antibody response out to 100 days or greater. Once a classification has been made, it can be used in the selection and benchmarking of vaccines for therapeutic use. The classification may further include selection of an agent or regimen.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “adjuvant” generally refers to a composition that increases the humoral or cellular immune response of an individual. Adjuvants of interest stimulate the immune system, and increase responsiveness or durability of response to a co-administered antigen.

The terms “subject,” “individual,” and “patient” are used interchangeably herein to refer to a mammal being assessed for response. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc. The methods of the invention can be applied for veterinary purposes.

As used herein, the term “theranosis” refers to the use of results obtained from a diagnostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any that provides information on the state of a disease, condition, or symptom.

The terms “therapeutic agent”, “therapeutic capable agent” or “treatment agent” are used interchangeably and refer to a molecule or compound that confers some beneficial effect upon administration to a subject, including vaccines and vaccine adjuvants. The beneficial effect includes induction of a therapeutic immune response, enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular response, which cellular response may be mediated by antigen-specific T cells or their secretion products), and innate immune cells. An “immunogen” is capable of inducing an immunological response against itself on administration to a mammal or due to autoimmune disease.

The term “vaccine”, as used herein, is defined in accordance with the pertinent art and relates to a composition that induces or enhances the protective immunity of an individual to a particular disease caused by a pathogen. Without wishing to be bound by theory, it is believed that a protective immunity arises from the generation of neutralizing antibodies, or from the activation of cytotoxic cells of the immune system, or both. In order to induce or enhance a protective immunity, a vaccine comprises as an immunogenic antigen a part of the pathogen causing said disease or a nucleic acid molecule encoding this immunogenic antigen. Upon contact with the immunogenic antigen, the immune system of the individual is triggered to recognise the immunogenic antigen as foreign and to destroy it. The immune system subsequently remembers the contact with this immunogenic antigen, so that at a later contact with the disease-causing pathogen an easy and efficient recognition and destruction of the pathogen is ensured.

Vaccines known and used in the art include, for example, inactivated pathogen vaccines; live-attenuated pathogen vaccines; messenger RNA (mRNA) vaccines; subunit, recombinant, polysaccharide, and conjugate vaccines; toxoid vaccines; and viral vector vaccines. Inactivated vaccines use a killed version of the pathogen that causes a disease, e.g. Hepatitis A, influenza, rabies, etc. Live vaccines use an attenuated form of the pathogen that causes a disease, e.g. measles, mumps, rubella (MMR combined vaccine), rotavirus, smallpox, chickenpox, yellow fever. mRNA vaccines encode pathogen proteins that trigger an immune response, e.g. SRS-CoV2. Subunit, recombinant, polysaccharide, and conjugate vaccines use specific pathogen molecules, e.g. Hib (Haemophilus influenzae type b), Hepatitis B, HPV (Human papillomavirus), Bordetella pertussis, pneumococcal disease, meningococcal disease, Varivella Zoster virus. Toxoid vaccines use a toxin made by the pathogen, e.g. Diphtheria, and tetanus. Viral vector vaccines use a modified version of a different virus as a vector to deliver sequences encoding pathogen protein. Several different viruses have been used as vectors, including influenza, vesicular stomatitis virus (VSV), measles virus, and adenovirus. Viral vectors are in use currently for SARS-COV2 vaccination.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers include expression levels of a gene of interest. Markers can also include combinations of any one or more of the foregoing measurements, including temporal trends and differences. Broadly used, a marker can also refer to an immune cell subset.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a sample comprising circulating immune cells. A sample can include, without limitation, an aliquot of body fluid, whole blood, PBMC (white blood cells or leucocytes), tissue biopsies, synovial fluid, lymphatic fluid, ascites fluid, and interstitial or extracellular fluid. “Blood sample” can refer to whole blood or a fraction thereof, including blood cells, white blood cells or leucocytes. Samples can be obtained from a subject by means including but not limited to venipuncture, biopsy, needle aspirate, lavage, scraping, surgical incision, or intervention or other means known in the art.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

“Affinity reagent”, or “specific binding member” may be used to refer to an affinity reagent, such as a polynucleotide, antibody, ligand, etc. that selectively binds to a genetic sequence, protein or marker of the invention. The term “affinity reagent” includes any molecule, e.g., peptide, nucleic acid, small organic molecule. In some embodiments, the affinity reagent is a polynucleotide

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′,F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of pre-existing conditions. For example, the development of immunity can be accomplished by administration of the agent. The treatment of ongoing disease, where the treatment stabilizes or improves the clinical symptoms of the patient, is of particular interest.

### Methods of the Invention

Analysis, at a single cell level or multiple cell level, of cellular biological samples obtained from an individual is used to obtain a determination of changes in immune cell gene expression associated with immunization. It is surprisingly found that changes occurring in gene expression of these immune cells is predictive of the propensity to develop a durable antibody response to a vaccine. In some embodiments, the immune cells are platelets.

The sample can be any suitable type that allows for the analysis of one or more cells, preferably a blood sample, PBMC sample, or fractions thereof, e.g. platelets. Samples can be obtained once or multiple times from an individual. Multiple samples can be obtained from different locations in the individual (e.g., blood samples, bone marrow samples and/or lymph node samples), at different times from the individual, or any combination thereof. In an embodiment, a baseline, or “day 0” sample is obtained prior to immunization, and a test sample is obtained from about 7 to about 10 days following immunization, e.g. at about day 7, about day 8, about day 9, about day 10, and may be from about day 6 to about 11, from about 7 about 10, from about 7 to about 9, from about 7 to about 8 days.

When samples are obtained as a series, e.g., a series of blood samples obtained during, the samples can be obtained at fixed intervals, at intervals determined by the status of the most recent sample or samples or by other characteristics of the individual, or some combination thereof. It will be appreciated that an interval may not be exact, according to an individual's availability for sampling and the availability of sampling facilities, thus approximate intervals corresponding to an intended interval scheme are encompassed by the invention. Generally, the most easily obtained samples are fluid samples. In some embodiments the sample or samples is blood.

One or more cells or cell types, or samples containing one or more cells or cell types, can be isolated from body samples. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells can be obtained. Alternatively, a heterogeneous cell population can be used, e.g. circulating peripheral blood mononuclear cells.

In some embodiments of the invention, different gating strategies are used in order to analyze a specific cell population (e.g., only CD4⁺ T cells, only platelets, etc.) in a sample of mixed cell population. These gating strategies can be based on the presence of one or more specific surface markers. The following gate can differentiate between dead cells and live cells and the subsequent gating of live cells classifies them into, e.g. myeloid blasts, monocytes and lymphocytes. A clear comparison can be carried out by using two-dimensional contour plot representations, two-dimensional dot plot representations, and/or histograms.

Samples may be obtained at one or more time points. Where a sample at a single time point is used, comparison is made to a reference “base line” level for the presence of the activated form of the signaling protein of interest, which may be obtained from a normal control, a pre-determined level obtained from one or a population of individuals, from a negative control for ex vivo activation, and the like.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells can be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In an embodiment, a method is provided for predicting the durability of an immune response to a candidate vaccine, the method comprising administering the candidate vaccine, which may comprise an adjuvant, to a mammal; and determining the RNA content of platelets in the recipient following vaccination.

In some embodiments, analysis of platelet RNA content is performed in a one-step flow cytometry analysis, e.g. by fluorescence activated flow cytometry. In such methods, a sample, e.g. a peripheral blood sample, is labeled with reagents that can distinguish platelets from other cells in the sample; and labeled with an RNA selective stain. The population of cells is then analyzed by flow cytometry and gated on the platelet population to determine the RNA contnt of platelets. The cells can be fresh or frozen, and may be fixed prior to analysis.

In such embodiments, a sample from an individual is contacted with one or a cocktail of directly or indirectly labeled binding agents, e.g. labeled antibodies, that are specific for markers that can distinguish platelets. In some embodiments, the binding agents are specific for CD41 and CD61, where platelets are CD41⁺CD61⁺. In some embodiments, the cocktail of binding agents further comprises an agent specific for one or more of CD3, CD8, CD14, CD19, CD20, CD56, etc., which markers are used to exclude non-platelet cells from analysis. For example, a cocktail of antibodies for staining may comprise detectable labeled anti-CD3, anti-CD19, anti-CD14, anti-CD56, anti-CD41, and anti-CD61 antibodies. Another cocktail of antibodies may comprise anti-CD3, anti-CD8, anti-CD20, anti-CD14, anti-CD41, and anti-CD61 antibodies.

Alternatively, a blood sample can be anticoagulated to obtain platelet-rich plasma, where the plaelet rich plasma is contacted with one or a cocktail of directly or indirectly labeled binding agents, e.g. labeled antibodies, that are specific for markers that can distinguish platelets. In some embodiments, the binding agents are specific for CD41 and CD61, where platelets are CD41⁺CD61⁺. In some embodiments, the cocktail of binding agents further comprises an agent specific for one or more of a red blood cell marker, including without limitation TER119, which marker is used to exclude non-platelet RBC from analysis. For example, a cocktail of antibodies for staining may comprise detectable labeled anti-TER119, anti-CD41, and anti-CD61 antibodies.

The sample is contacted with a dye that is selective for RNA. Suitable dyes for this purpose are commercially available, e.g. RNASelect™ Stain (Invitrogen), which exhibits bright green fluorescence when bound to RNA (absorption/emission maxima ˜490/530 nm), but only a weak fluorescent signal when bound to DNA. Other RNA selective dyes are known in the art, for example styryl dyes E36, E144 and F22, described by Li et al. (2006) Chemistry and Biology 13 (6): 615-623, herein specifically incorporated by reference; and RNA-selective fluorescent dye integrated with a thiazole orange and a p-(methylthio) styryl moiety, described by Lu et al. Chemical Communications 215 (83).

The sample is then analyzed by flow cytometry by gating on the CD41⁺CD61⁺ platelets, optionally excluding RBC and other immune cells, and determining the platelet RNA content. The fold change in platelet RNA content can be compared to the baseline level prior to vaccination, where a durable immune response is associated with an increase of at least about 5-fold, at least about 10-fold, at least about 20-fold relative to baseline.

### Data Analysis

A signature pattern can be generated from a biological sample using any convenient protocol, for example as described below. The readout can be a mean, average, median or the variance or other statistically or mathematically-derived value associated with the measurement, e.g. gene expression, RNA content, etc. The marker readout information can be further refined by direct comparison with the corresponding reference or control pattern. A signature can be evaluated on a number of points: to determine if there is a statistically significant change at any point in the data matrix relative to a reference value; whether the change is an increase or decrease in the binding; whether the change is specific for one or more physiological states, and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a classification regarding the response of the patient from which the sample was obtained/derived. Additionally, a reference or control signature pattern can be a signature pattern that is obtained from a sample of a reference adjuvant.

In certain embodiments, the obtained signature pattern is compared to a single reference/control profile to obtain information regarding the phenotype. In yet other embodiments, the obtained signature pattern is compared to two or more different reference/control profiles to obtain more in depth information. For example, the obtained signature pattern can be compared to a positive and negative reference profile to obtain confirmed information.

Samples can be obtained from the tissues or fluids of an individual. For example, samples can be obtained from whole blood, tissue biopsy, serum, etc. Also included in the term are derivatives and fractions of such cells and fluids

In order to identify profiles, a statistical test can provide a confidence level for a change in the level of markers between the test and reference profiles to be considered significant. The raw data can be initially analyzed by measuring the values for each marker, usually in duplicate, triplicate, quadruplicate or in 5-10 replicate features per marker. A test dataset is considered to be different than a reference dataset if one or more of the parameter values of the profile exceeds the limits that correspond to a predefined level of significance.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients. The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation. Alternatively, any convenient method of statistical validation can be used.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T2 statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These tools and methods can be applied to several classification problems. For example, methods can be developed from the following comparisons: i) all cases versus all controls, ii) all cases versus non-durable response, iii) all cases versus durable response.

In a second analytical approach, variables chosen in the cross-sectional analysis are separately employed as predictors. Given the specific outcome, the random lengths of time each patient will be observed, and selection of proteomic and other features, a parametric approach to analyzing responsiveness can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models can be used, especially since reductions of numbers of covariates to manageable size with the lasso will significantly simplify the analysis, allowing the possibility of an entirely nonparametric approach to survival.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

“Antigen” or “immunogen” refers to any substance that stimulates an immune response. The term includes killed, inactivated, attenuated, or modified live bacteria, viruses, or parasites. The term antigen also includes polynucleotides, polypeptides, recombinant proteins, synthetic peptides, protein extract, cells (including bacterial cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

“Cellular immune response” or “cell mediated immune response” is one mediated by T-lymphocytes or other white blood cells or both, and includes the production of cytokines, chemokines and similar molecules produced by activated T-cells, white blood cells, or both.

“Emulsifier” means a substance used to make an emulsion more stable.

“Emulsion” means a composition of two immiscible liquids in which small droplets of one liquid are suspended in a continuous phase of the other liquid.

“Immune response” in a subject refers to the development of a humoral immune response, a cellular immune response, or a humoral and a cellular immune response to an antigen. Immune responses can usually be determined using standard immunoassays and neutralization assays, which are known in the art.

“Immunogenic” means evoking an immune or antigenic response. Thus an immunogenic composition would be any composition that induces an immune response.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

“Reactogenicity” refers to the side effects elicited in a subject in response to the administration of an adjuvant, an immunogenic, or a vaccine composition. It can occur at the site of administration, and is usually assessed in terms of the development of a number of symptoms. These symptoms can include inflammation, redness, and abscess. It is also assessed in terms of occurrence, duration, and severity. A “low” reaction would, for example, involve swelling that is only detectable by palpitation and not by the eye, or would be of short duration. A more severe reaction would be, for example, one that is visible to the eye or is of longer duration.

“Immunostimulatory composition” refers to a composition that includes an adjuvant, as defined herein and may optionally further include an antigen, in which case it may be more conventionally referred to as a vaccine. Administration of the composition to a subject results in an increased responsive state of myeloid immune cells. The amount of a composition that is therapeutically effective may vary depending on the presence of antigen, the adjuvant, and the condition of the subject, and can be determined by one skilled in the art. A non-antigenic adjuvant composition does not comprise an antigen for the disease of interest.

### Adjuvant Compositions

In some embodiments an adjuvant composition is selected for use or further development. Exemplary adjuvants are oil in water emulsions, and may comprise squalene in the oil phase. For example, AS03 is an adjuvant system composed of α-tocopherol, squalene and polysorbate 80 in an oil-in-water emulsion. MF59 is another immunologic adjuvant that comprises a squalene emulsion. The dose of adjuvant administered may depend on whether an antigen is present, on the antigen with which it is used and the antigen dosage to be applied. It is also dependent on the intended species and the desired formulation. Usually the quantity is within the range conventionally used for adjuvants. For example, adjuvants typically comprises from about 1 μg to about 1000 μg, inclusive, of a 1-mL dose.

The adjuvant formulations can be homogenized or microfluidized. The formulations are subjected to a primary blending process, typically by passage one or more times through one or more homogenizers. Any commercially available homogenizer can be used for this purpose, e.g., Ross emulsifier (Hauppauge, N.Y.), Gaulin homogenizer (Everett, Mass.), or Microfluidics (Newton, Mass.). In one embodiment, the formulations are homogenized for three minutes at 10,000 rpm. Microfluidization can be achieved by use of a commercial mirofluidizer, such as model number 110Y available from Microfluidics, (Newton, Mass.); Gaulin Model 30CD (Gaulin, Inc., Everett, Mass.); and Rainnie Minilab Type 8.30H (Miro Atomizer Food and Dairy, Inc., Hudson, Wis.). These microfluidizers operate by forcing fluids through small apertures under high pressure, such that two fluid streams interact at high velocities in an interaction chamber to form compositions with droplets of a submicron size. In one embodiment, the formulations are microfluidized by being passed through a 200 micron limiting dimension chamber at 10,000+/−500 psi.

The routes of administration for the adjuvant compositions include parenteral, oral, oronasal, intranasal, intratracheal, topical, etc. Any suitable device may be used to administer the compositions, including syringes, droppers, needleless injection devices, patches, and the like. The route and device selected for use will depend on the composition of the adjuvant, the antigen, and the subject, and such are well known to the skilled artisan.

The adjuvant compositions can further include one or more immunomodulatory agents such as, e.g., quaternary ammonium compounds (e.g., DDA), and interleukins, interferons, or other cytokines. These materials can be purchased commercially. The amount of an immunomodulator suitable for use in the adjuvant compositions depends upon the nature of the immunomodulator used and the subject. However, they are generally used in an amount of about 1 μg to about 5,000 μg per dose. For a specific example, adjuvant compositions containing DDA can be prepared by simply mixing an antigen solution with a freshly prepared solution of DDA.

The adjuvant compositions can further include one or more polymers such as, for example, DEAE Dextran, polyethylene glycol, and polyacrylic acid and polymethacrylic acid (eg, CARBOPOL®). Such material can be purchased commercially. The amount of polymers suitable for use in the adjuvant compositions depends upon the nature of the polymers used. However, they are generally used in an amount of about 0.0001% volume to volume (v/v) to about 75% v/v. In other embodiments, they are used in an amount of about 0.001% v/v to about 50% v/v, of about 0.005% v/v to about 25% v/v, of about 0.01% v/v to about 10% v/v, of about 0.05% v/v to about 2% v/v, and of about 0.1% v/v to about 0.75% v/v. In another embodiment, they are used in an amount of about 0.02 v/v to about 0.4% v/v. DEAE-dextran can have a molecular size in the range of 50,000 Da to 5,000,000 Da, or it can be in the range of 500,000 Da to 2,000,000 Da. Such material may be purchased commercially or prepared from dextran.

The adjuvant compositions can further include one or more Th2 stimulants such as, for example, Bay R1005™ and aluminum. The amount of Th2 stimulants suitable for use in the adjuvant compositions depends upon the nature of the Th2 stimulant used. However, they are generally used in an amount of about 0.01 mg to about 10 mg per dose. In other embodiments, they are used in an amount of about 0.05 mg to about 7.5 mg per dose, of about 0.1 mg to about 5 mg per dose, of about 0.5 mg to about 2.5 mg per dose, and of 1 mg to about 2 mg per dose. A specific example is Bay R1005™, a glycolipid with the chemical name “N-(2-deoxy-2-L-leucylamino-β-D-glucopyranosyl)-N-octadecyldodecanamide acetate.” It is an amphiphilic molecule which forms micelles in aqueous solution.

Some examples of bacteria causing disease for which immune responsiveness may be obtained include, for example, Aceinetobacter calcoaceticus, Acetobacter paseruianus, Actinobacillus pleuropneumoniae, Aeromonas hydrophila, Alicyclobacillus acidocaldarius, Arhaeglobus fulgidus, Bacillus pumilus, Bacillus stearothermophillus, Bacillus subtilis, Bacillus thermocatenulatus, Bordetella bronchiseptica, Burkholderia cepacia, Burkholderia glumae, Campylobacter coli, Campylobacter fetus, Campylobacter jejuni, Campylobacter hyointestinalis, Chlamydia psittaci, Chlamydia trachomatis, Chlamydophila spp., Chromobacterium viscosum, Erysipelothrix rhusiopathieae, Listeria monocytogenes, Ehrlichia canis, Escherichia coli, Haemophilus influenzae, Haemophilus somnus, Helicobacter suis, Lawsonia intracellularis, Legionella pneumophilia, Moraxellsa sp., Mycobactrium bovis, Mycoplasma hyopneumoniae, Mycoplasma mycoides subsp. mycoides LC, Clostridium perfringens, Odoribacter denticanis, Pasteurella (Mannheimia) haemolytica, Pasteurella multocida, Photorhabdus luminescens, Porphyromonas gulae, Porphyromonas gingivalis, Porphyromonas salivosa, Propionibacterium acnes, Proteus vulgaris, Pseudomnas wisconsinensis, Pseudomonas aeruginosa, Pseudomonas fluorescens C9, Pseudomonas fluorescens SIKW1, Pseudomonas fragi, Pseudomonas luteola, Pseudomonas oleovorans, Pseudomonas sp B11-1, Alcaliges eutrophus, Psychrobacter immobilis, Rickettsia prowazekii, Rickettsia rickettsia, Salmonella typhimurium, Salmonella bongori, Salmonella enterica, Salmonella dublin, Salmonella typhimurium, Salmonella choleraseuis, Salmonella newport, Serratia marcescens, Spirlina platensis, Staphlyoccocus aureus, Staphylococcus epidermidis, Staphylococcus hyicus, Streptomyces albus, Streptomyces cinnamoneus, Streptococcus suis, Streptomyces exfoliates, Streptomyces scabies, Sulfolobus acidocaldarius, Syechocystis sp., Vibrio cholerae, Borrelia burgdorferi, Treponema denticola, Treponema minutum, Treponema phagedenis, Treponema refringens, Treponema vincentii, Treponema palladium, and Leptospira species, such as the known pathogens Leptospira canicola, Leptospira grippotyposa, Leptospira hardjo, Leptospira borgpetersenii hardjo-bovis, Leptospira borgpetersenii hardjo-prajitno, Leptospira interrogans, Leptospira icterohaemorrhagiae, Leptospira pomona, and Leptospira bratislava, and combinations thereof.

Examples of viruses causing disease for which immune responsiveness may be obtained include, for example, SARS-Cov1, SARS-Cov2, and other coronaviruses, Avian herpesviruses, Bovine herpesviruses, Canine herpesviruses, Equine herpesviruses, Feline viral rhinotracheitis virus, Marek's disease virus, Ovine herpesviruses, Porcine herpesviruses, Pseudorabies virus, Avian paramyxoviruses, Bovine respiratory syncytial virus, Canine distemper virus, Canine parainfluenza virus, canine adenovirus, canine parvovirus, Bovine Parainfluenza virus 3, Ovine parainfluenza 3, Rinderpest virus, Border disease virus, Bovine viral diarrhea virus (BVDV), BVDV Type I, BVDV Type II, Classical swine fever virus, Avian Leukosis virus, Bovine immunodeficiency virus, Bovine leukemia virus, Bovine tuberculosis, Equine infectious anemia virus, Feline immunodeficiency virus, Feline leukemia virus (FeLV), Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, virus, Porcine epidemic diarrhea virus, Porcine hemagglutinating encephalomyletitis virus, Porcine parvovirus, Porcine Circovirus (PCV) Type I, PCV Type II, Porcine Reproductive and Respiratory Syndrome (PRRS) Virus, Transmissible gastroenteritis virus, Turkey coronavirus, Bovine ephemeral fever virus, Rabies, Rotovirus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, and combinations thereof.

Examples of parasites causing disease for which immune responsiveness may be obtained include, for example, Anaplasma, Fasciola hepatica (liver fluke), Coccidia, Eimeria spp., Neospora caninum, Toxoplasma gondii, Giardia, Dirofilaria (heartworms), Ancylostoma (hookworms), Trypanosoma spp., Leishmania spp., Trichomonas spp., Cryptosporidium parvum, Babesia, Schistosoma, Taenia, Strongyloides, Ascaris, Trichinella, Sarcocystis, Hammondia, and Isopsora, and combinations thereof. Also contemplated are external parasites including, but not limited to, ticks, including Ixodes, Rhipicephalus, Dermacentor, Amblyomma, Boophilus, Hyalomma, and Haemaphysalis species, and combinations thereof.

Oil, when added as a component of an adjuvant, generally provides a long and slow release profile. In the present invention, the oil can be metabolizable or non-metabolizable. The oil can be in the form of an oil-in-water, a water-in-oil, or a water-in-oil-in-water emulsion.

Oils suitable for use in the present invention include alkanes, alkenes, alkynes, and their corresponding acids and alcohols, the ethers and esters thereof, and mixtures thereof. The individual compounds of the oil are light hydrocarbon compounds, i.e., such components have 6 to 30 carbon atoms. The oil can be synthetically prepared or purified from petroleum products. The moiety may have a straight or branched chain structure. It may be fully saturated or have one or more double or triple bonds. Some non-metabolizable oils for use in the present invention include mineral oil, paraffin oil, and cycloparaffins, for example.

The term oil is also intended to include “light mineral oil,” i.e., oil which is similarly obtained by distillation of petrolatum, but which has a slightly lower specific gravity than white mineral oil.

Metabolizable oils include metabolizable, non-toxic oils. The oil can be any vegetable oil, fish oil, animal oil or synthetically prepared oil which can be metabolized by the body of the subject to which the adjuvant will be administered and which is not toxic to the subject. Sources for vegetable oils include nuts, seeds and grains.

Other components of the compositions can include pharmaceutically acceptable excipients, such as carriers, solvents, and diluents, isotonic agents, buffering agents, stabilizers, preservatives, vaso-constrictive agents, antibacterial agents, antifungal agents, and the like. Typical carriers, solvents, and diluents include water, saline, dextrose, ethanol, glycerol, oil, and the like. Representative isotonic agents include sodium chloride, dextrose, mannitol, sorbitol, lactose, and the like. Useful stabilizers include gelatin, albumin, and the like.

Surfactants are used to assist in the stabilization of the emulsion selected to act as the carrier for the adjuvant and antigen. Surfactants suitable for use in the present inventions include natural biologically compatible surfactants and non-natural synthetic surfactants. Biologically compatible surfactants include phospholipid compounds or a mixture of phospholipids. Preferred phospholipids are phosphatidylcholines (lecithin), such as soy or egg lecithin. Lecithin can be obtained as a mixture of phosphatides and triglycerides by water-washing crude vegetable oils, and separating and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, and phosphatidylethanolamine. The phospholipids may be isolated from natural sources or conventionally synthesized.

Non-natural, synthetic surfactants suitable for use in the present invention include sorbitan-based non-ionic surfactants, e.g. fatty-acid-substituted sorbitan surfactants, fatty acid esters of polyethoxylated sorbitol (TWEEN™), polyethylene glycol esters of fatty acids from sources such castor fatty as oil; polyethoxylated acid, polyethoxylated isooctylphenol/formaldehyde polymer, polyoxyethylene fatty alcohol ethers (BRIJ™); polyoxyethylene nonphenyl ethers (TRITON™), polyoxyethylene isooctylphenyl ethers (TRITON™ X).

As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. The carrier(s) must be “acceptable” in the sense of being compatible with the other components of the compositions and not deleterious to the subject. Typically, the carriers will be will be sterile and pyrogen-free, and selected based on the mode of administration to be used. It is well known by those skilled in the art that the preferred formulations for the pharmaceutically acceptable carrier which comprise the compositions are those pharmaceutical carriers approved in the applicable regulations promulgated by the United States (US) Department of Agriculture or US Food and Drug Administration, or equivalent government agency in a non-US country. Therefore, the pharmaceutically accepted carrier for commercial production of the compositions is a carrier that is already approved or will be approved by the appropriate government agency in the US or foreign country.

The compositions optionally can include compatible pharmaceutically acceptable (i.e., sterile or non-toxic) liquid, semisolid, or solid diluents that serve as pharmaceutical vehicles, excipients, or media. Diluents can include water, saline, dextrose, ethanol, glycerol, and the like. Isotonic agents can include sodium chloride, dextrose, mannitol, sorbitol, and lactose, among others. Stabilizers include albumin, among others.

The compositions can also contain antibiotics or preservatives, including, for example, gentamicin, merthiolate, or chlorocresol. The various classes of antibiotics or preservatives from which to select are well known to the skilled artisan.

Kits may be provided. Kits may further include cells or reagents suitable for isolating and culturing cells in preparation for conversion; reagents suitable for culturing T cells; and reagents useful for determining the epigenomic effect of a vaccine adjuvant. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### System Biological Analysis of Vaccination Adjuvanted with AS03 Reveals Mechanisms of Adjuvanticity and Antibody Durability in Humans

In order to elucidate the molecular mechanisms by which AS03 induces robust and durable immune responses in humans, here we performed an in-depth multi-omics analysis of cellular, transcriptional, and metabolic responses to a prepandemic H5N1 avian influenza vaccine with and without AS03 in a cohort of healthy adults. Through a meta-analysis of influenza vaccine datasets, we were able to identify a key set of genes induced specifically by AS03. Pathway analysis of these genes suggests a critical role for apoptosis in AS03's mechanism of action. Furthermore, plasma metabolomics analysis revealed that AS03-induced perturbations in lipid and fatty acid metabolism were highly associated with expression of the AS03-specific apoptotic signature. We were able to establish an early gene signature capable of successfully predicting antibody response longevity in an independent blinded cohort of H5N1+AS03 vaccinees. Subsequent single cell profiling revealed differences in RNA content among platelets as a major driver of this cell adhesion-related longevity signature. Together, these results provide important insight into the mechanisms by which the pandemic adjuvant AS03 can induce potent and lasting immunity to vaccination and can help guide targeted development of novel adjuvants to improve vaccines against future pandemics.

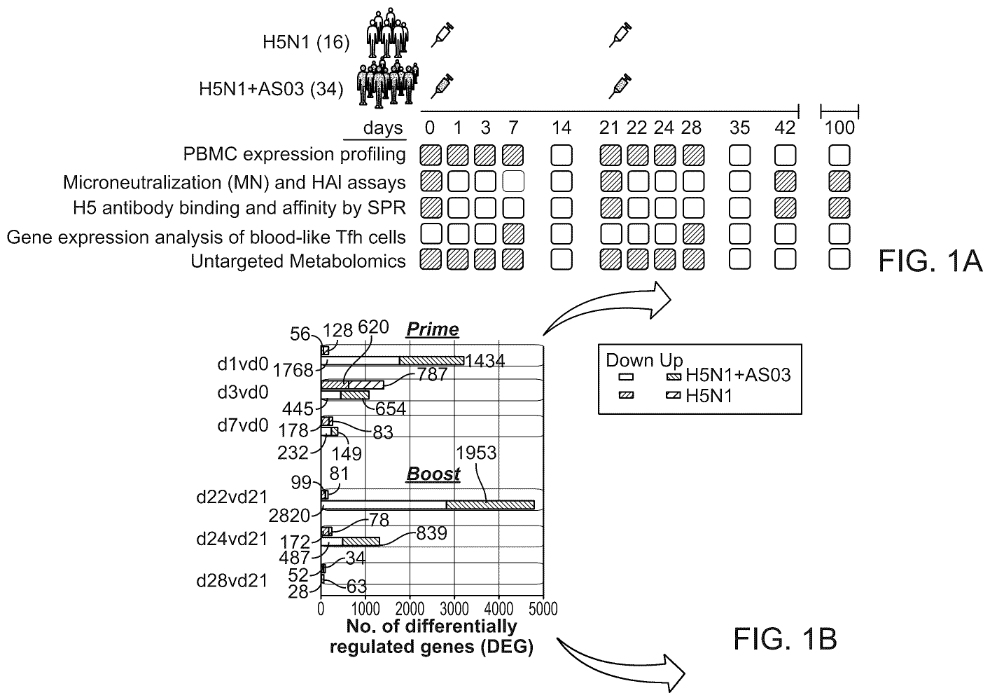

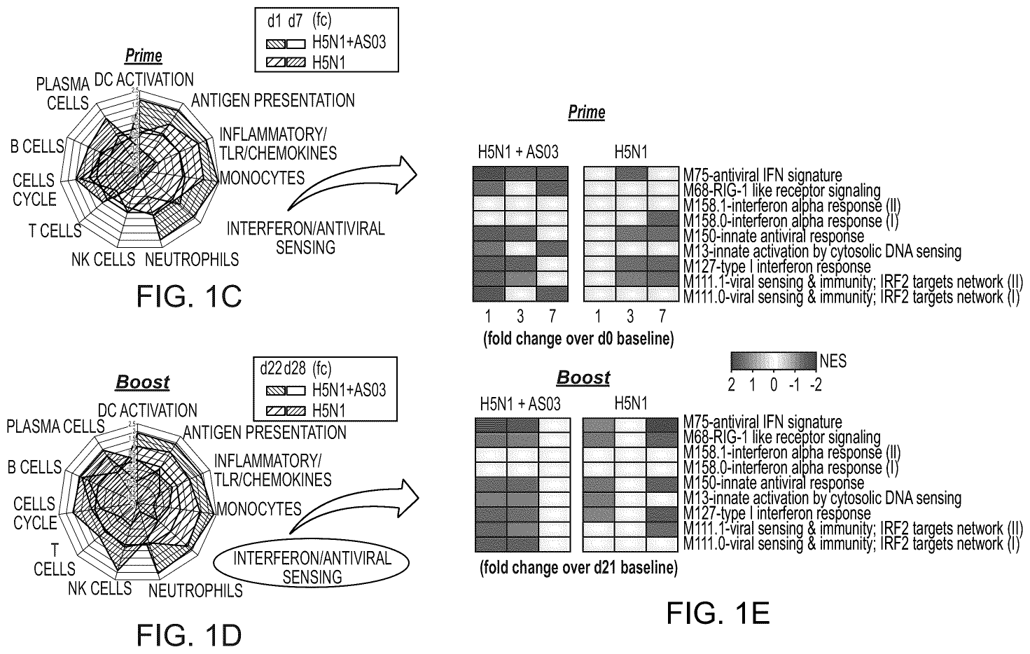

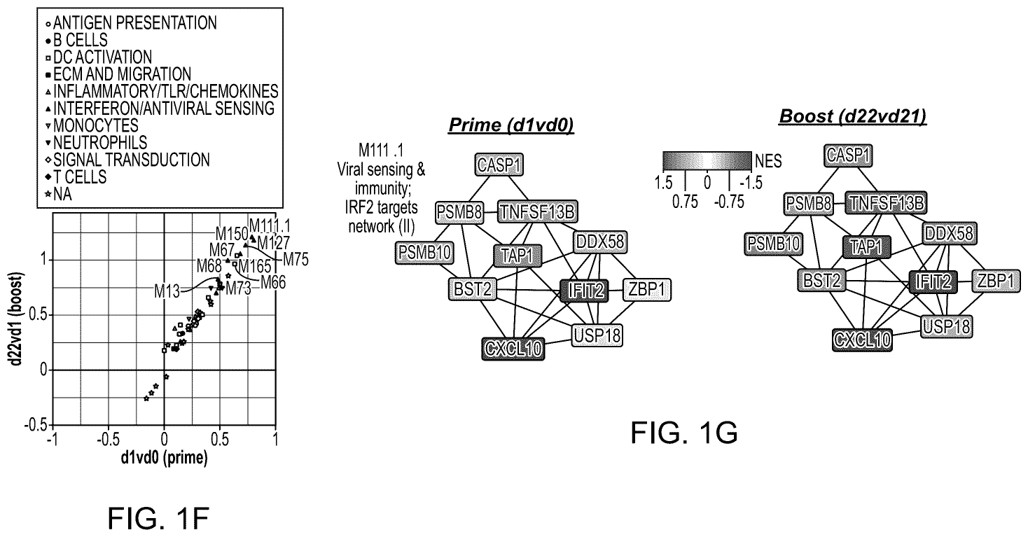

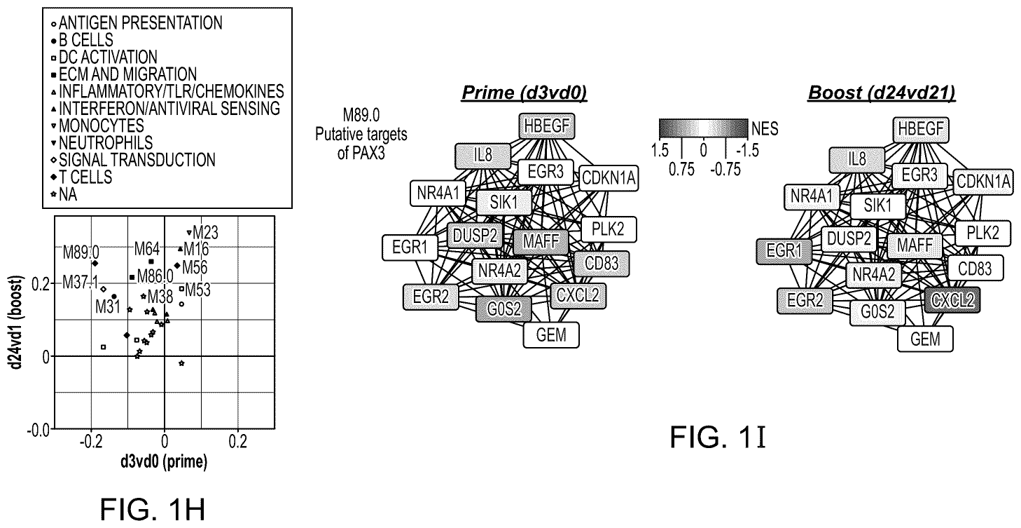

AS03 induces potent early transcriptional signatures which are enhanced after a booster vaccination. We randomized in a 2:1 ratio a total of 50 healthy subjects aged 21-45 years old to receive two doses 21 days apart of a monovalent, split-virion, inactivated H5N1 clade 2.1 A/Indonesia/05/2005 influenza vaccine, administered with (n=34) or without (n=16) the AS03 adjuvant (FIG. 1A and Supplemental Table 1). In order to obtain a comprehensive picture of innate and adaptive immune responses to adjuvanted versus unadjuvanted H5N1 vaccination, we performed gene expression analyses on peripheral blood mononuclear cells (PBMCs) and measured key immunological parameters of vaccine induced B- and T-cell responses on the first day of vaccination and at regular intervals thereafter, as illustrated in FIG. 1A.

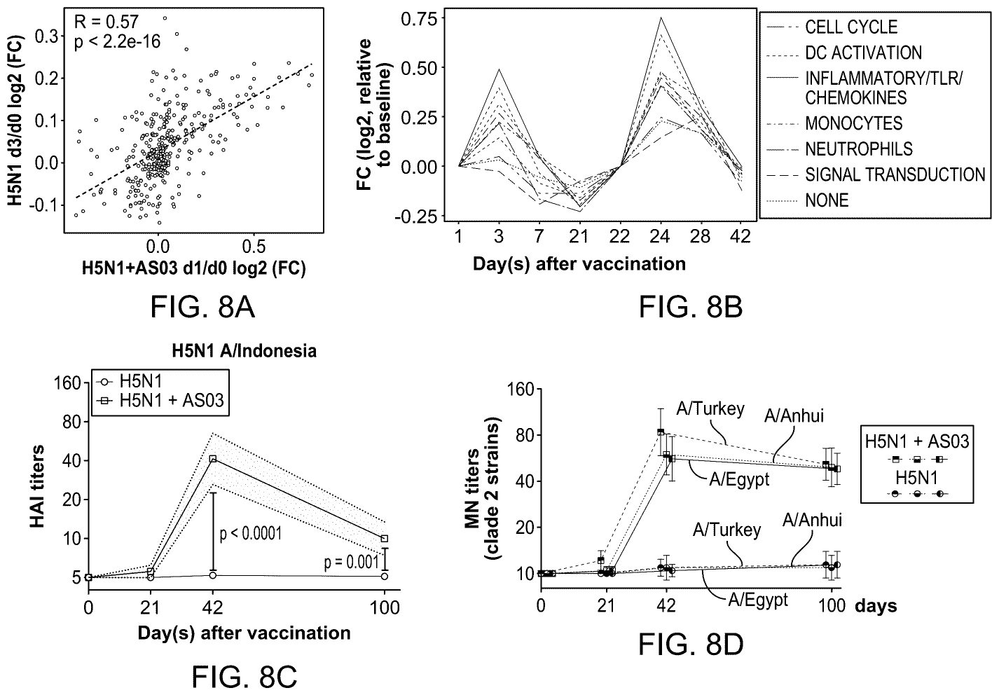

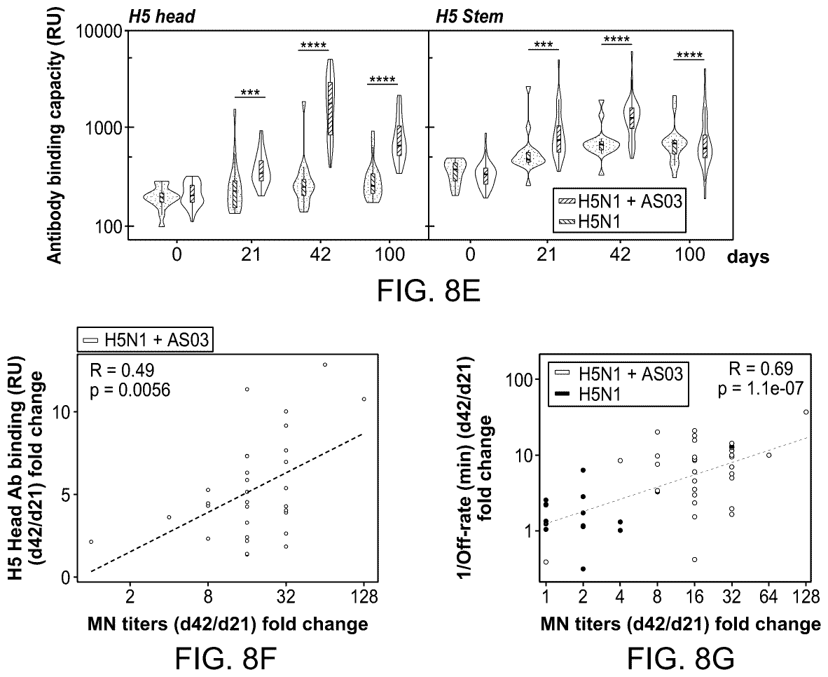

We started our investigation by examining the impact of AS03 on gene expression in PBMCs. First, we identified differentially expressed genes (DEGs) post-vaccination in both the adjuvanted and unadjuvanted groups. H5N1+AS03 induced a much more robust transcriptional response than H5N1 alone, with the large majority of DEGs observed at day 1 post-prime and boost (FIG. 1B). By examining the overlap in DEGs between both groups at different time points, we observed that the peak response in the unadjuvanted group at day 3 post prime shared many genes with day 1 prime and boost responses in the adjuvanted group (FIG. 8A), indicating that the kinetics of the response is delayed in unadjuvanted subjects.

To identify the specific pathways activated in response to H5N1 vaccination, we performed gene set enrichment analysis (GSEA) on genes ranked by post-vaccination fold change. As a basis for this analysis, we used a set of blood transcriptional modules (BTMs) previously identified by our group through large-scale network integration of publicly available human blood transcriptomes. Merging BTM enrichment scores according to high-level functional categories revealed that AS03 increased expression of a broad range of innate and adaptive immune cells and pathways on day 1 and 7 post-prime and boost vaccination, respectively (FIGS. 1C and 1D). Particularly, we found strong enrichment of BTMs related to monocyte and dendritic cell (DC) activation at early time-points after each immunization, while day 7 responses were mostly dominated by robust B cell and plasma cell transcriptional responses. On the other hand, unadjuvanted subjects showed very little immune activation after the prime dose, and required a boost dose to achieve modest enrichment of innate responses such as antigen presentation and interferon signaling, as detailed in FIG. 1E. Accordingly, B cell and plasma cell modules were only mildly modulated in the unadjuvanted group 7 days after prime (FIG. 1C), but more strongly regulated after boost (FIG. 1D), although to a lesser extent than the adjuvant group.

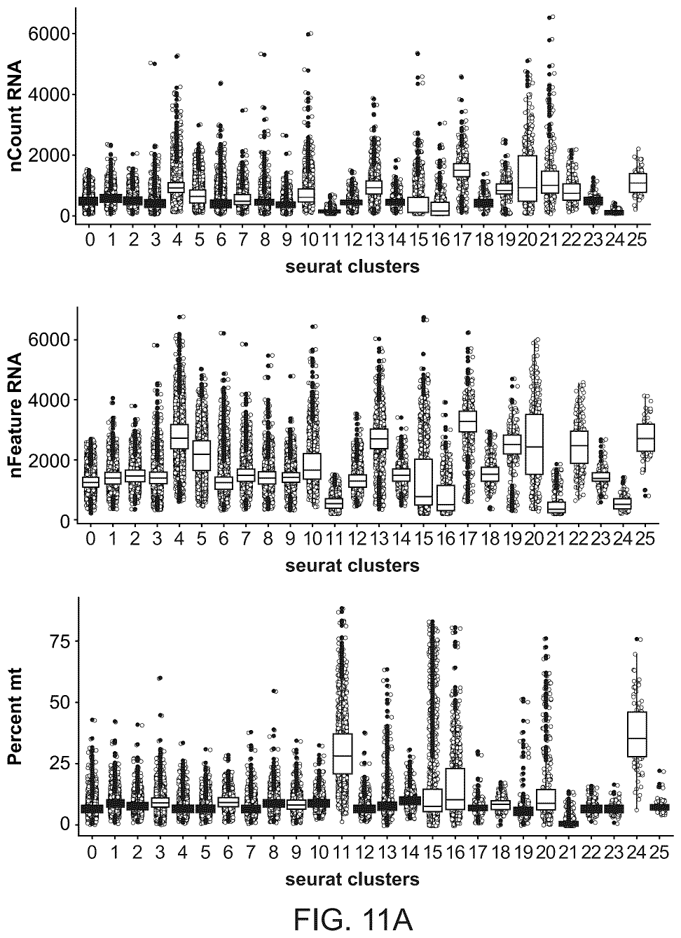

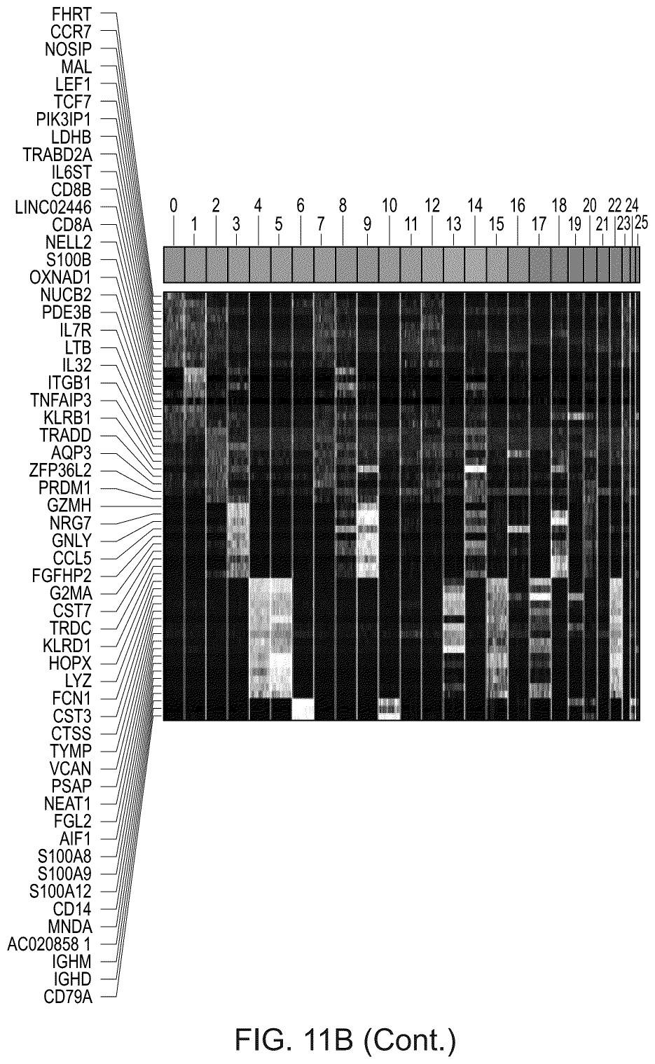

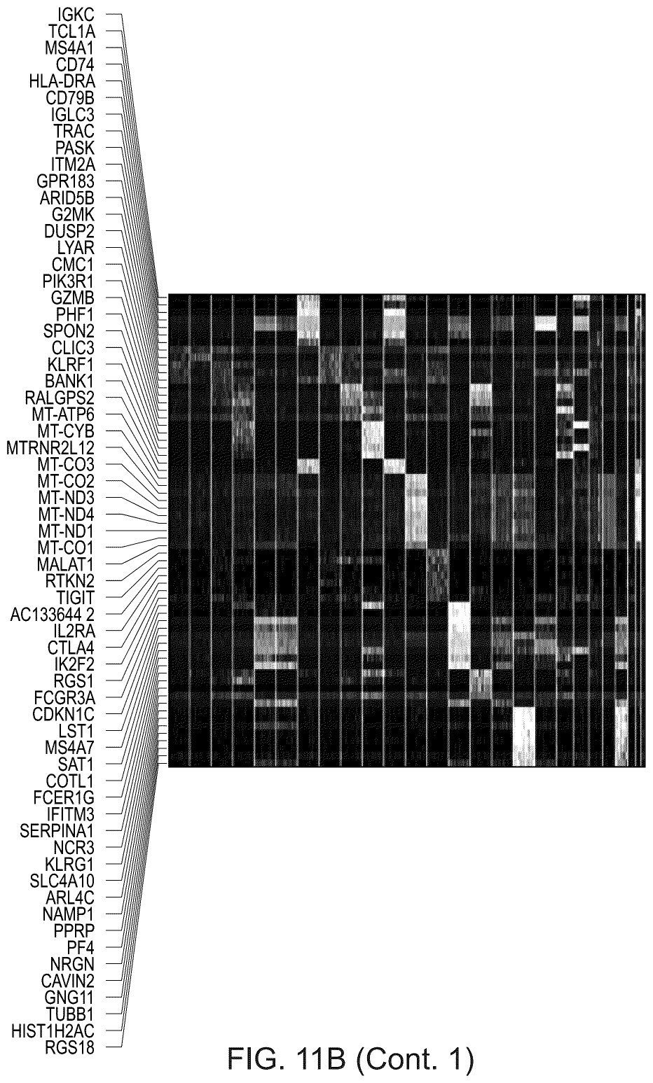

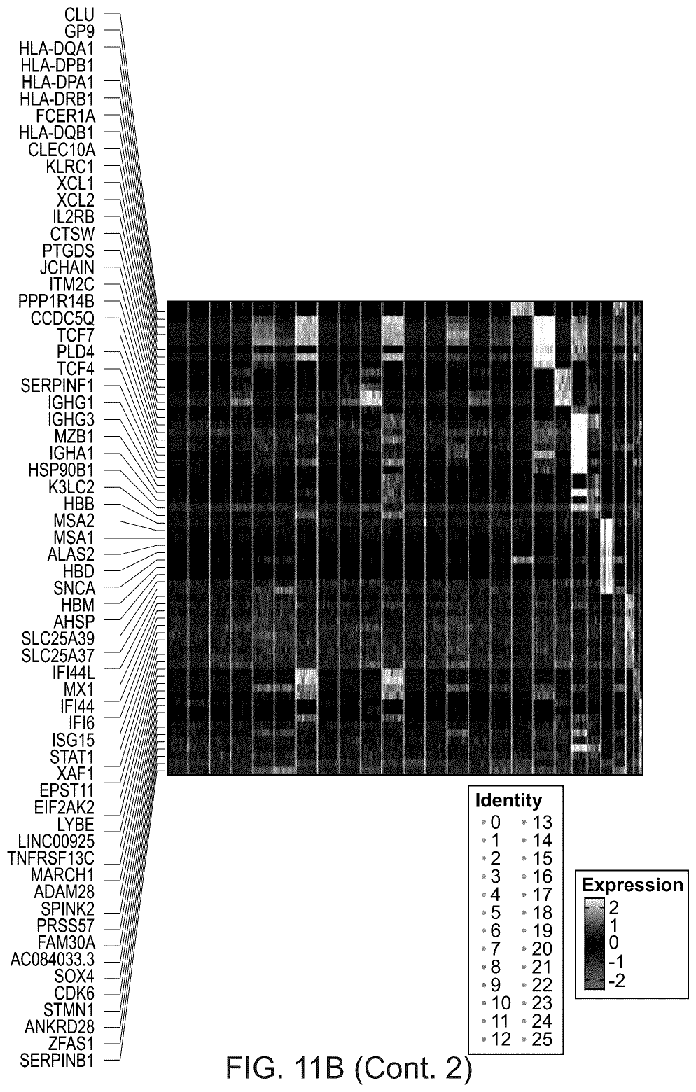

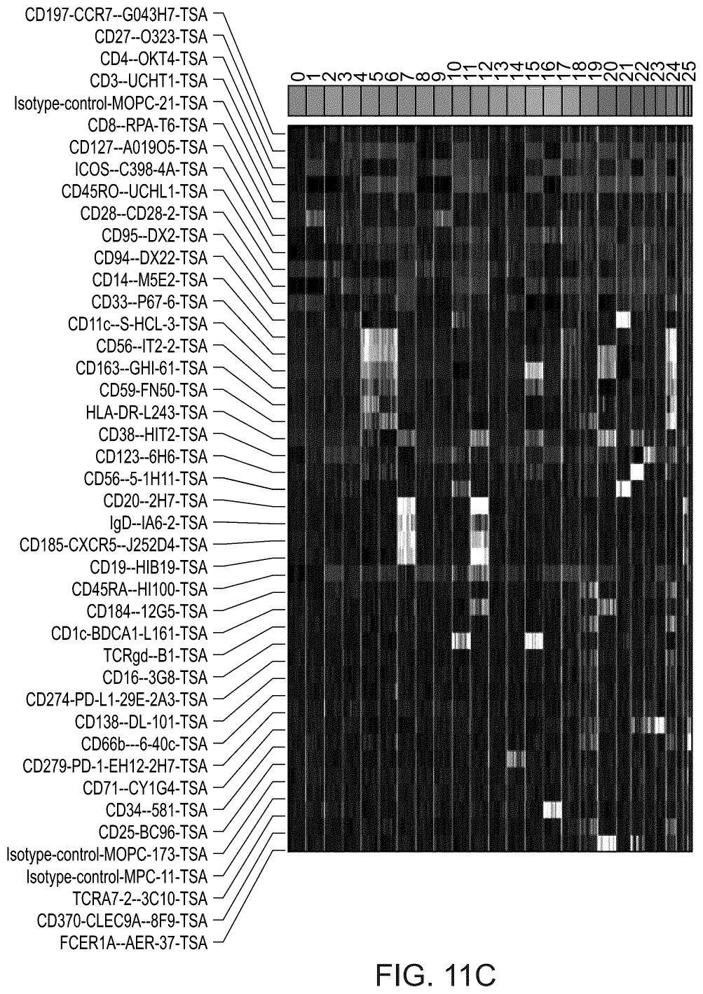

Notably, while analyzing the effect of AS03 on gene expression, we noticed significant differences in transcriptional activity after each dose of adjuvanted vaccine. Indeed, we found several BTMs related to interferon signaling and DC activation to be more strongly upregulated on day 1 after the booster dose (d22) compared to day 1 post prime (FIGS. 1F and 1G), despite a broad similarity overall in the type of immune responses elicited by AS03 after each dose. Gene expression analysis on day 3 and day 24 responses, however, revealed more qualitative differences between prime and boost immunizations, with multiple BTMs negatively enriched after the first dose but positively enriched after a second vaccination with AS03 (FIG. 1H). Importantly, genes under the control of the transcriptional factor PAX3 encoding for molecules with important chemotactic functions for the recruitment of granulocytes, monocytes, and macrophages such as IL-8 and HBEGF, appeared downregulated 3 days post prime but still robustly upregulated on day 3 after boost (FIG. 11 and FIG. 8B), thus suggesting previously unappreciated implications for the prime-boost regimen with AS03 on the quality, magnitude, and persistence of innate immune responses to H5N1 vaccination.

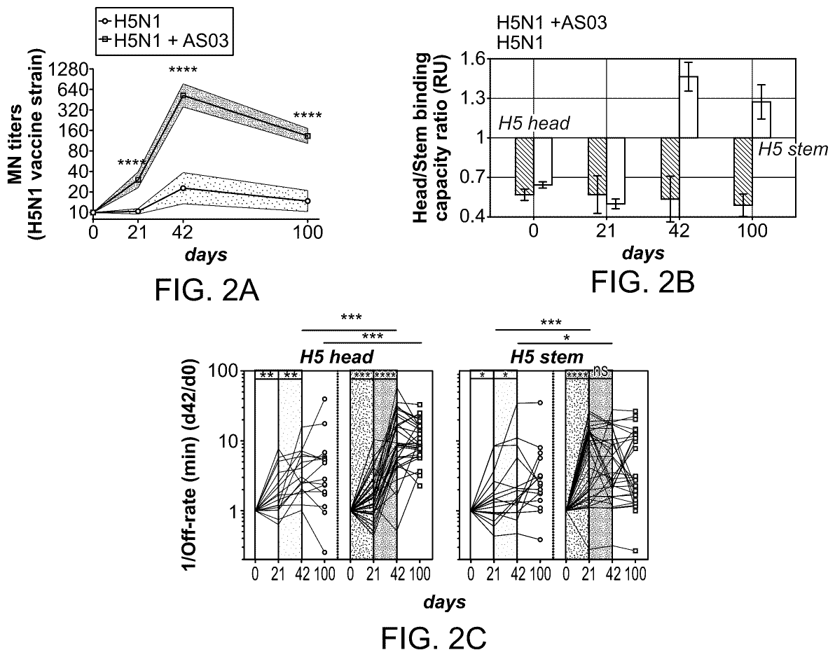

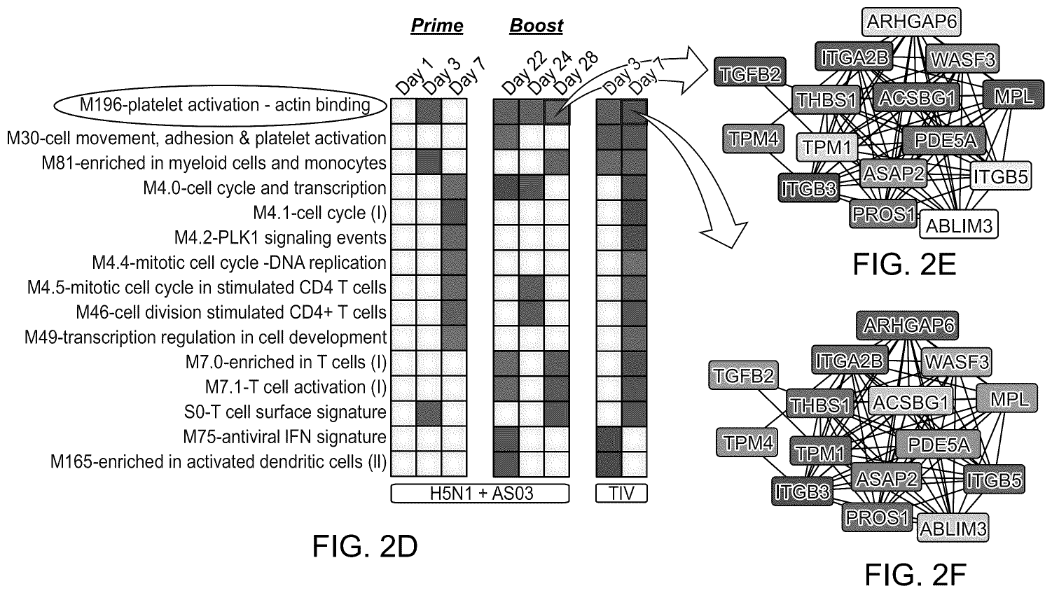

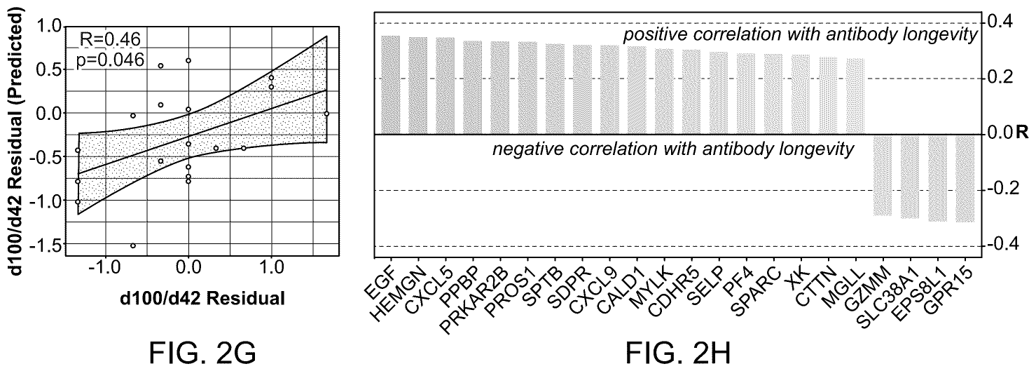

Adjuvanted H5N1 vaccination promotes protective H5-head directed antibody responses whose durability is associated with a transcriptional signature of cellular migration. AS03 has been previously reported to enhance antibody responses in humans in the context of influenza vaccination. Accordingly, we observed a significant increase in H5N1 A/Indonesia-specific microneutralization (MN) titers at every measured time-point following immunization with AS03 (FIG. 2A). A booster vaccination resulted in a 70% seroconversion rate by day 42, as determined by HAI titers (FIG. 8C and Supplemental Table 2). In contrast, subjects in the unadjuvanted group showed significantly lower MN and HAI responses, with no vaccine recipient achieving a 4-fold increase in HAI titers over baseline levels. In addition to inducing neutralizing antibodies against the vaccine strain, AS03-adjuvanted vaccination promoted broad cross-clade neutralizing antibodies against three heterologous H5N1 strains belonging to clade 2, namely clade 2.2.1 A/Turkey, clade 2.2.1 A/Egypt, and clade 2.3.4 A/Anhui (FIG. 8D), but not against a clade 1 A/Vietnam strain, indicating that AS03 induces broad antibody-mediated cross-protection against phylogenetically close, but not distant H5N1 viruses.

Furthermore, we used surface plasmon resonance (SPR) real time kinetics assays to quantitate total antibody binding and polyclonal sera antibody affinity against recombinant HA1 (head) and HA2 (stem) domains derived from the boosting H5N1 vaccine strain. SPR measurements showed that subjects who received the AS03-adjuvanted H5N1 vaccine exhibited significantly higher levels of HA-binding antibodies to both H5 head and stem subunits after both prime and boost when compared with subjects in the unadjuvanted group (FIG. 8E). Importantly, while both cohorts generated antibodies mostly targeting the H5 stem domain after the prime immunization, H5 head: stem antibody ratio changed drastically in the AS03 group following the second immunization, with a substantial increase in the concentration of polyclonal antibodies directed against the H5 head domain that persisted over time (FIG. 2B) and correlated with MN titers (FIG. 8F). In contrast, study participants who received H5N1 alone generated only a marginal increase in their concentration of H5 head-specific antibodies following vaccination, with antibody responses in this vaccine group mostly confined to a moderate increase in H5 stem-specific antibody titers. Further, dissociation rates of antigen-antibody complexes revealed that, although comparable to the unadjuvanted group after prime, antibody affinity for the H5 head domain was significantly higher in the AS03 group post boost (FIG. 2C) and correlated with MN titers (FIG. 8G). Predictably, H5N1 alone was able to induce only limited antibody affinity maturation for the H5 head and stem domains (FIG. 2C), thus suggesting recurrence of a memory recall response rather than epitope spreading of the antibody repertoire.

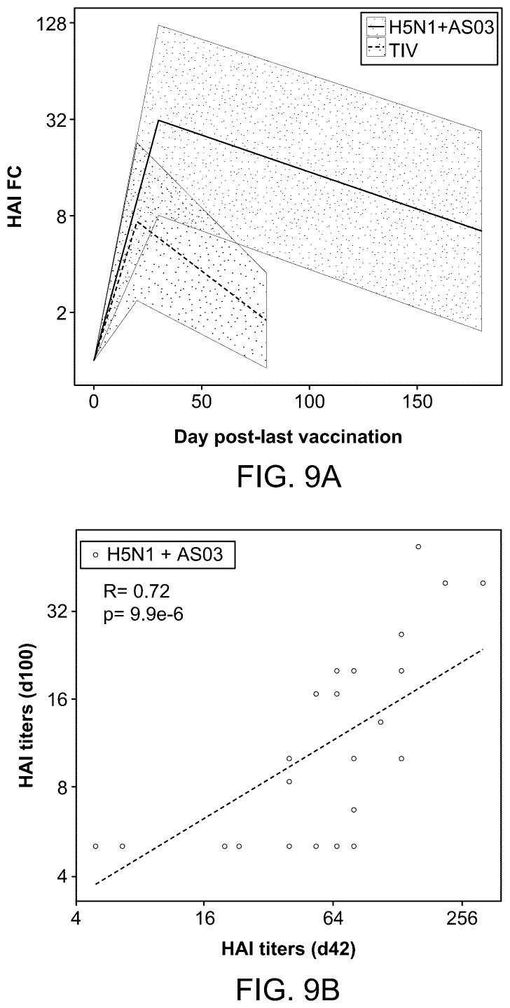

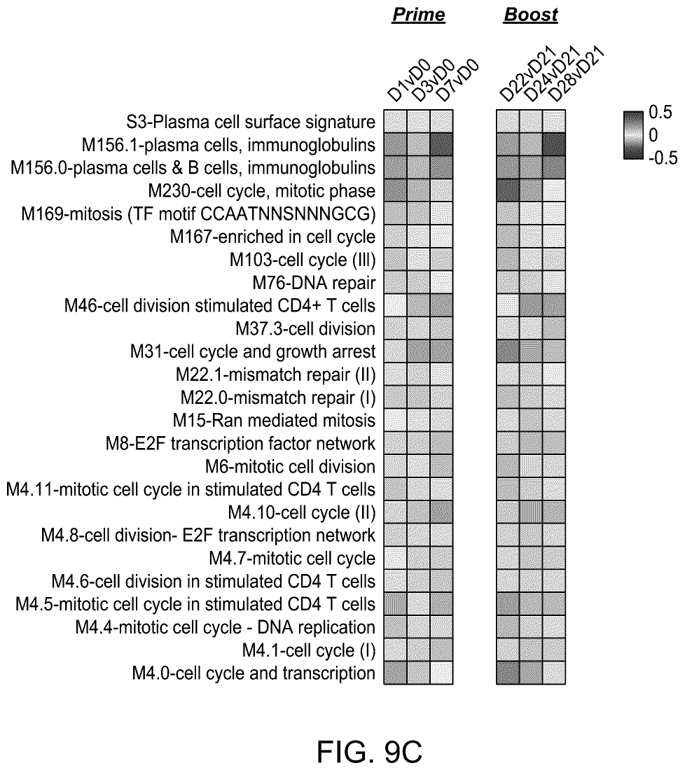

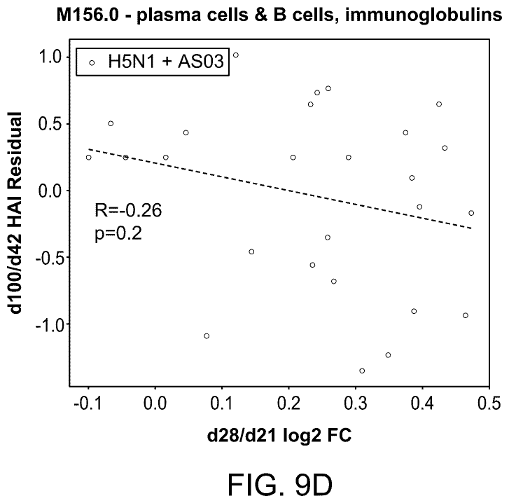

The recent COVID-19 crisis has reinforced the importance to develop pandemic vaccines that could induce long-lasting protection, particularly in a scenario where multiple epidemic waves might occur and vaccine demand might exceed supply, as in the early phases of a pandemic onset. Here we observed a four-fold decrease in geometric mean MN and HAI titers between day 42 and day 100 post-prime H5N1+AS03 vaccination (FIG. 2A and FIG. 8C). A comparison between antibody responses to H5N1+AS03 and those to TIV showed that antibody titers against avian influenza exhibited a similar reduction in magnitude within several months post-vaccination to those against seasonal influenza strains (FIG. 9A). As a measure of the relative persistence of antibody responses, we calculated residuals from a linear fit between the day 42 and day 100 HAI titers (FIG. 9B). This approach removes the dependence of the day 100 titers on the initial day 42 response, and the residual can be viewed as a ‘normalized’ day 100 titer, with positive values indicating subjects with a more persistent response than average.

First, we determined whether the day 7 “plasmablast signature” observed by us and others in previous studies with seasonal influenza vaccination which was shown to be a correlate of HAI titers at day 28, was correlated with durability of antibody response. As seen previously in our study with seasonal flu vaccine, we did not observe a correlation of this with durability (Supplemental FIGS. 2C and 2D).

In order to identify transcriptional signatures associated with a persistent antibody response, we performed GSEA on genes ranked by their correlation with the day 100/day 42 residual (FIG. 2D). The enriched signatures appeared to differ between doses, with expression of cell cycle-related modules on day 7 post-prime and cell adhesion/platelet activation-related modules on days 1-7 post-boost being associated with increased persistence. We compared these results with previously identified signatures of persistence in TIV vaccination and found that there was significant overlap (FIG. 2D). In particular, genes within the platelet activation/actin binding module M196 showed strong agreement in their correlations with antibody persistence in both studies (FIGS. 2E and 2F), indicating a common mechanism in the development of long-lived antibody responses to influenza vaccination.

Further, to validate the robustness of these signatures of antibody persistence, we used a machine learning approach to train a classifier capable of predicting antibody persistence in an independent blind test set. In brief, we trained a linear regression model based on BTM-level features to predict the day 100/42 HAI residual, using the H5N1+AS03 data as a training set and the 2010/2011 TIV data as a validation set (see Methods for details). Using day 28/21 gene expression data, we were able to build a model whose predicted day 100/42 HAI residuals significantly correlated with the measured residuals in the blind test set (FIG. 2G). Notably, analysis of the genes within these predictive modules showed that many of those most highly associated with antibody persistence were involved in cellular adhesion/migration (CTTN, CDHR5, MYLK) and/or highly expressed in platelets (SELP, PROS1, PF4, PPBP) (FIG. 2H), indicating a potential role for cellular migration and platelets in the establishment of durable antibody responses to AS03-adjuvanted vaccination.

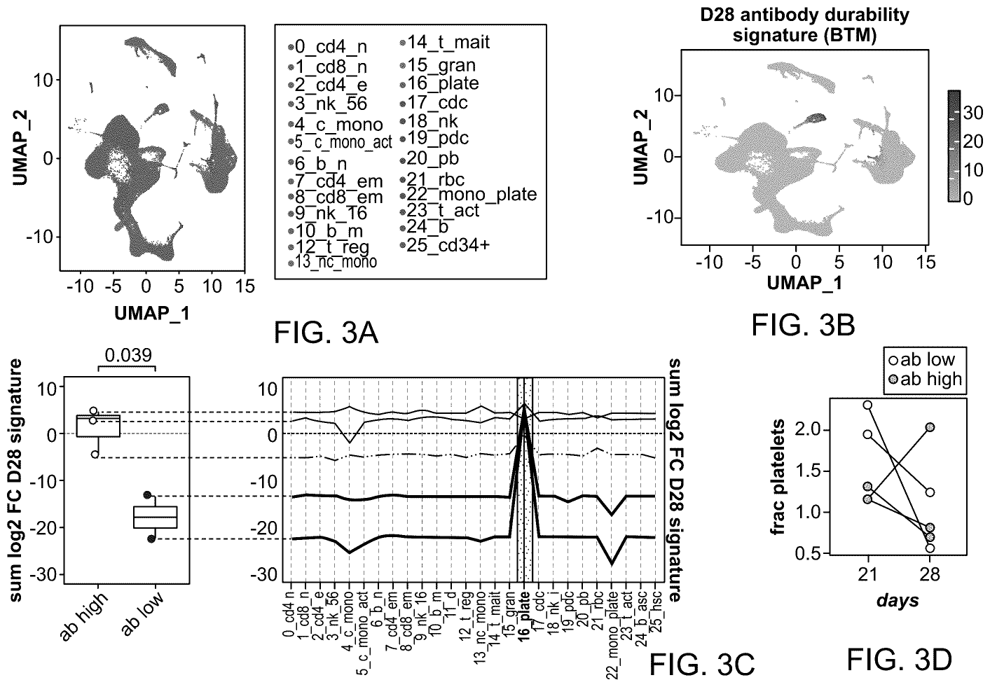

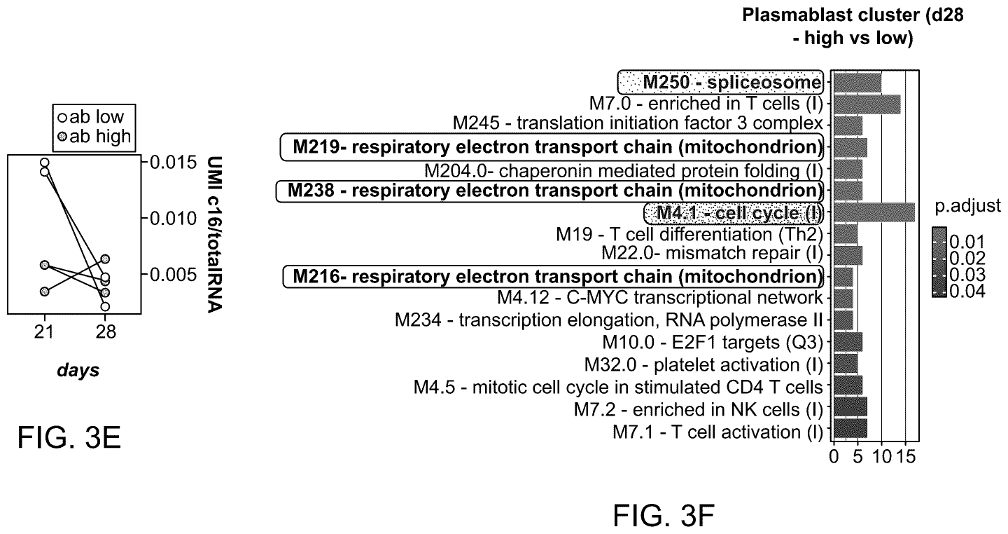

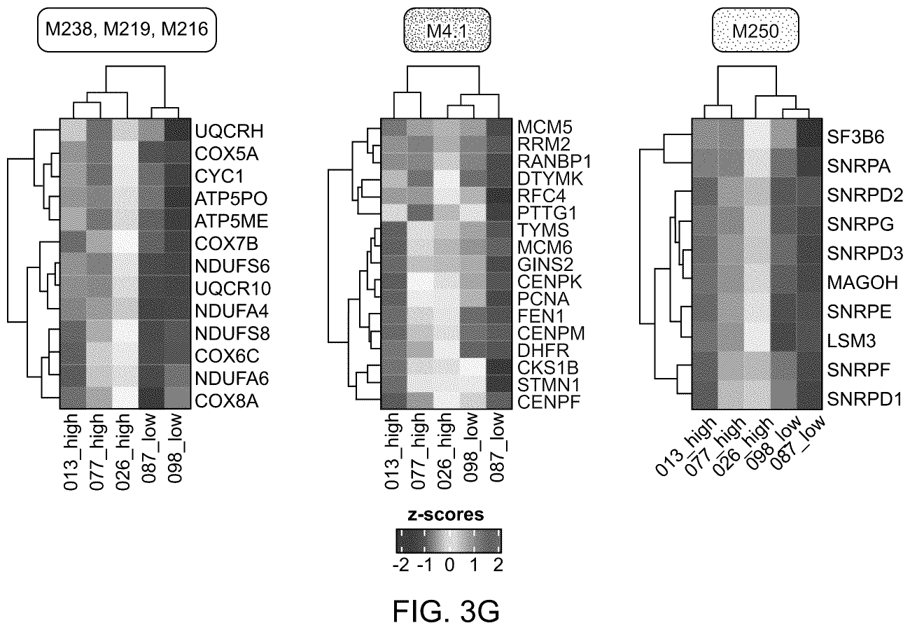

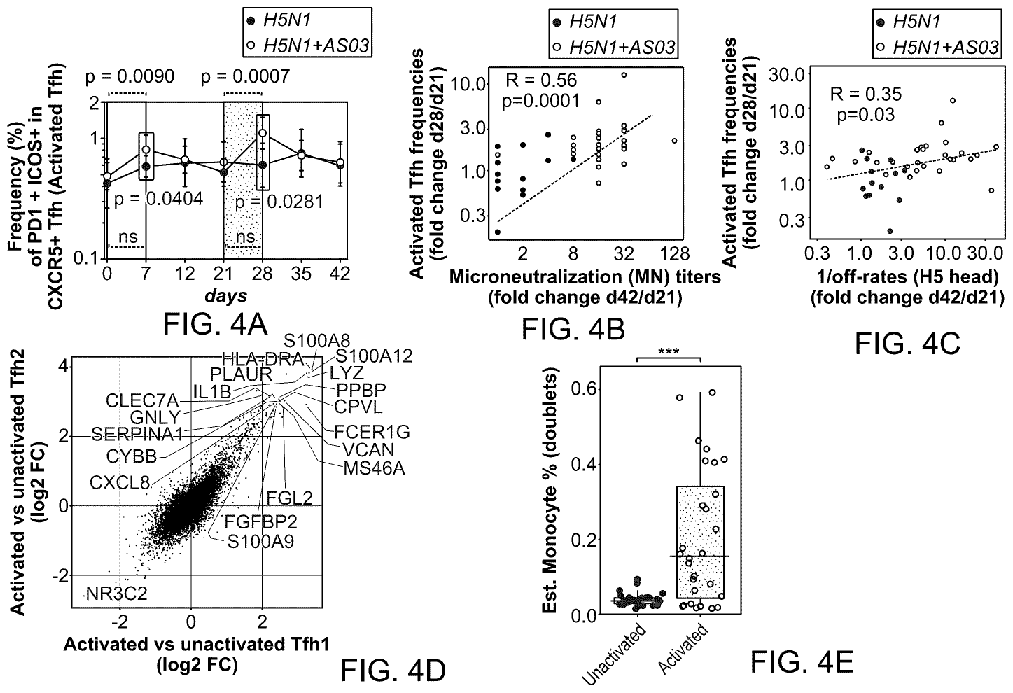

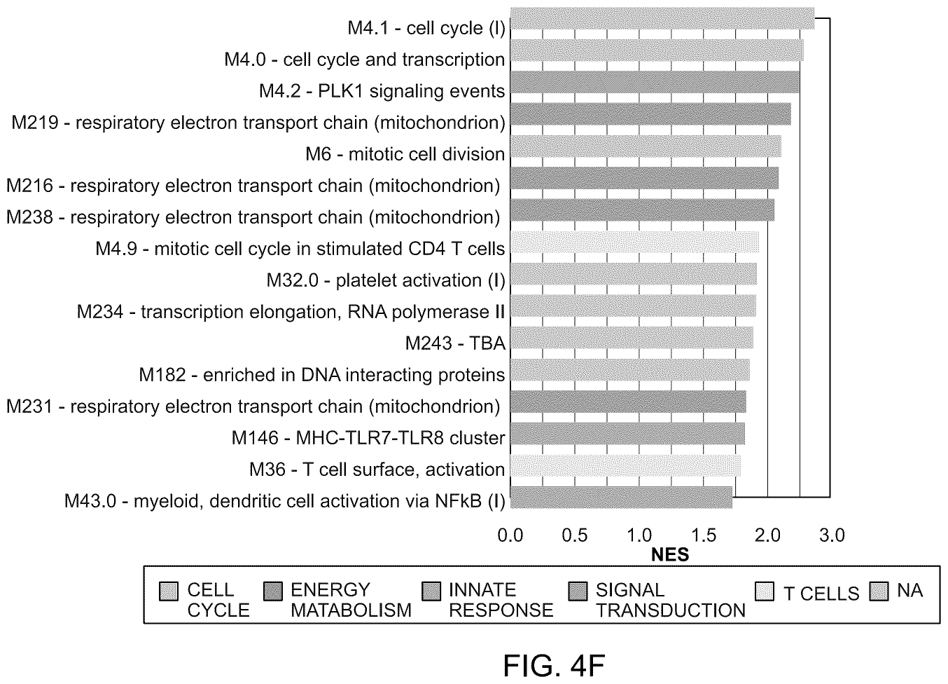

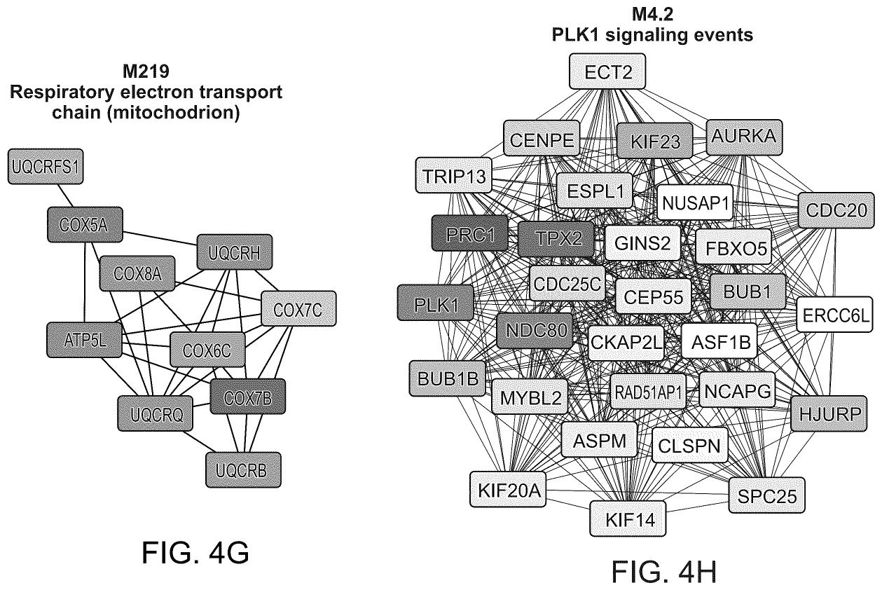

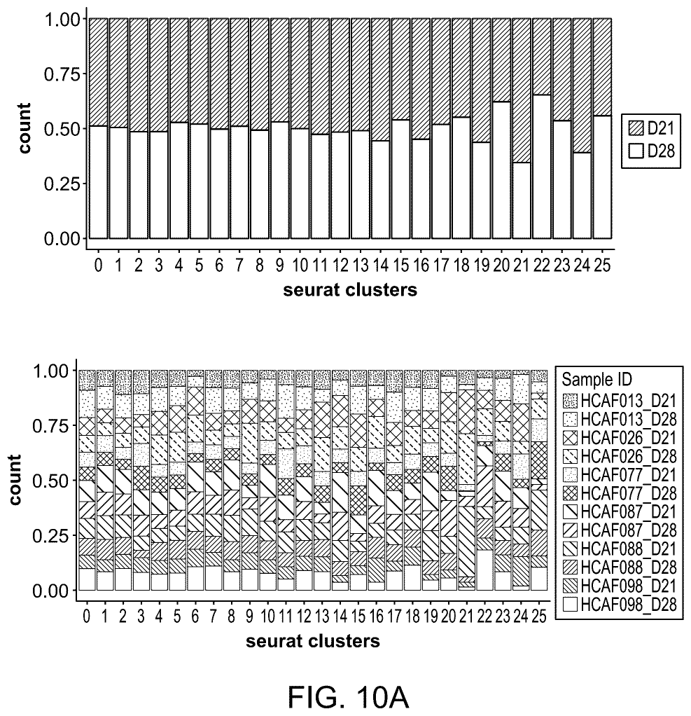

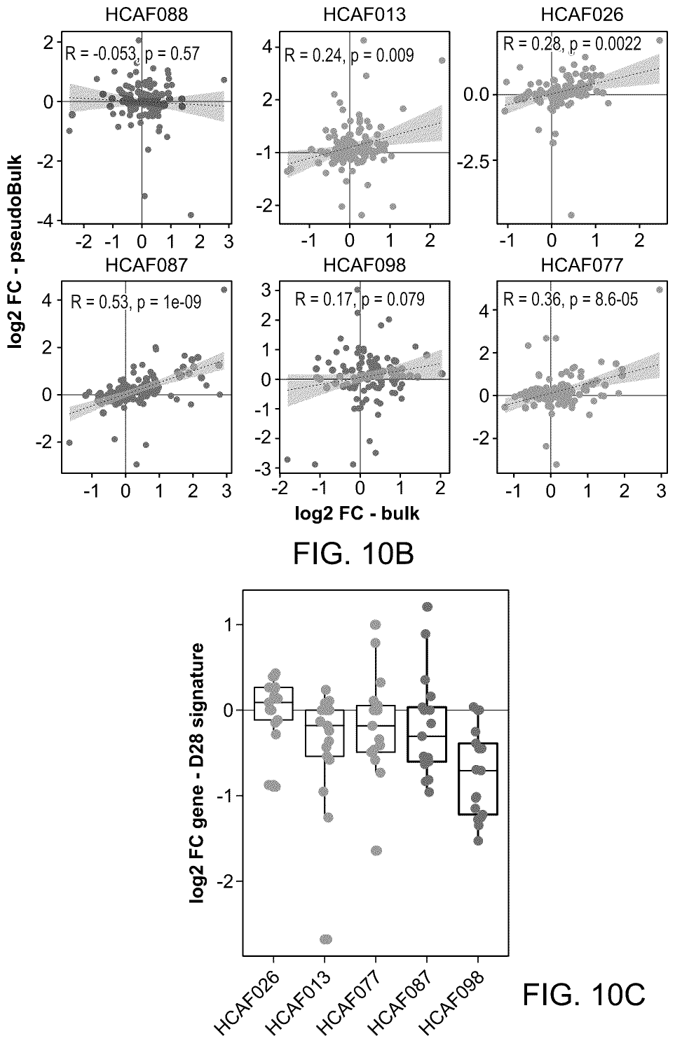

CITEseq analysis reveals a platelet origin for transcriptional signature of antibody persistence. Prompted by our findings, we sought to examine the cellular origins of this newly identified transcriptional signature of antibody persistence. To accomplish this, we performed CITE-seq (cellular indexing of transcriptomes and epitopes by sequencing) and constructed the single-cell protein and transcriptome landscape of PBMCs on day 21 and day 28 from 3 ‘persistent’ and 3 ‘waning’ antibody responders to H5N1+AS03 vaccination (day 100/42 HAI residual >0 and <0, respectively). After initial preprocessing, we obtained transcriptomes for 62,789 cells. Through dimensionality reduction via UMAP and graph-based clustering (see Methods for details), we were able to identify 26 distinct cell clusters which were evenly distributed over all samples and time points (FIG. 10A). Following cell-type annotation and per cell quality control processing to filter out low quality cells (see Methods section and Supplemental FIGS. 4A-C), we constructed the single immune cell landscape (FIG. 3A) and found that the antibody longevity-related genes in the predictive signature (as reported in FIG. 2H) were indeed highly expressed in platelets, as previously suggested by bulk PBMC gene expression analysis, and in a small cluster of cells that shared characteristics of both monocytes and platelets (FIG. 3B).

Next, we set out to investigate how the differences in the predictive gene signature between persistent and waning responders emerged at the single cell level. To ensure that the CITE-seq analysis captured the same immune response dynamics as the microarray data, we compared day 28/21 FCs of DEGs measured by microarray with pseudobulk estimates from CITE-seq (FIG. 10B). In this comparison, 5 subjects showed strong correlations between the two measurements, while 1 subject exhibited no agreement and was therefore removed from downstream analysis. Overall, in accordance with the bulk PBMC microarray results, persistent responders had significantly higher pseudobulk expression of the antibody durability signature compared to waning responders (FIG. 3C, left panel). Importantly, by removing 1 cell cluster at a time from the pseudobulk calculation, we found that exclusion of the platelet cluster from the analysis resulted in an almost complete loss of the transcriptional differences observed between persistent and waning antibody responders (FIG. 3C, right panel). This ‘leave-one-out approach’ demonstrated once again that the durability signature observed in the bulk PMBC analysis was almost exclusively originating from platelets.

However, we found no clear difference in platelet frequencies between persistent and waning responders (FIG. 3D). Additionally, when comparing per-cell normalized expression within the platelet population, we did not observe a significant difference in the day 28/21 gene signature between the two groups (FIG. 10C). In contrast, we recorded a large decrease in total reads within the platelet population specifically among waning responders between day 21 and day 28 that did not occur among persistent responders (FIG. 3E), thus indicating a reduction in RNA content within platelets, but not platelet frequencies, as the major source for the transcriptional differences captured in the antibody durability signature between persistent and waning responders.

Beyond identifying the cellular origins of the predictive antibody durability signature, we also asked whether we could identify additional cell-specific differences between persistent and waning responders that were not initially detected via bulk expression measurements. To this end, we performed an unbiased comparison and determined the DEGs between cells from persistent and waning responders at each time point and within each cluster followed by an overrepresentation analysis for genes included in the BTMs. As the primary cell type responsible for antibody responses to vaccination, of particular interest were differences among plasmablasts in spliceosome and electron transport expression (FIG. 3F). Comparing the genes within these modules, we observed that plasmablasts of persistent responders had elevated expression of genes encoding for small nuclear ribonucleoproteins as well as mitochondrial respiratory electron transport complexes compared to waning responders (FIG. 3G). These results are consistent with recent findings which indicate oxidative phosphorylation and mitochondrial remodeling as an important metabolic process necessary for B cell activation and antibody production by plasmablasts.

Frequency of vaccine-induced T follicular helper cells in blood correlates with neutralizing antibody titers and antibody avidity. The generation of neutralizing antibodies with high affinity to the H5 antigen promoted by AS03 prompted us to investigate the role of T follicular helper (Tfh) cells in the context of H5N1 vaccination. Tfh cells are crucial for affinity maturation of B cells in the germinal center (GC) and have been previously monitored following immunization with AS03 in animal models. Although bone fide GC Tfh cells are not commonly detected in peripheral blood, we measured a population of circulating Tfh-like CXCR5+ CD4+ T cells previously described to be functionally similar to GC Tfh cells before and after H5N1 vaccination. We found that immunization with H5N1+AS03 resulted in increased frequencies of activated, PD-1+ ICOS+, blood Tfh cells on day 7 day after each immunization (FIG. 4A). Strikingly, the increase in activated Tfh observed after boost was consistent and correlated with increase in both MN titers (FIG. 4B) and antibody affinity for the H5 head domain (FIG. 4C). On the contrary, we detected no significant change in activated Tfh cell frequencies following vaccination in the unadjuvanted group.

To further characterize vaccine-induced blood Tfh cells, we FACS-sorted PD-1+ ICOS+ CXCR5+ CD4+ activated- and PD-1-ICOS-quiescent CXCR5+ CD4+ T cells on days 7 and 28 and profiled their gene expression using Clariom S technology. Additionally, since previous studies had described the ability of CXCR3 and CCR6 markers in discriminating between peripheral non-efficient Tfh1 (CXCR3+ CCR6−) and efficient Tfh2 (CXCR3-CCR6−) or Tfh17 (CXCR3-CCR6+) cells based on their B cell helper potential (Schmitt et al., 2014), we sought to explore whether Tfh cell polarization could explain the differences in activated Tfh frequencies observed between the two vaccine groups after immunization.

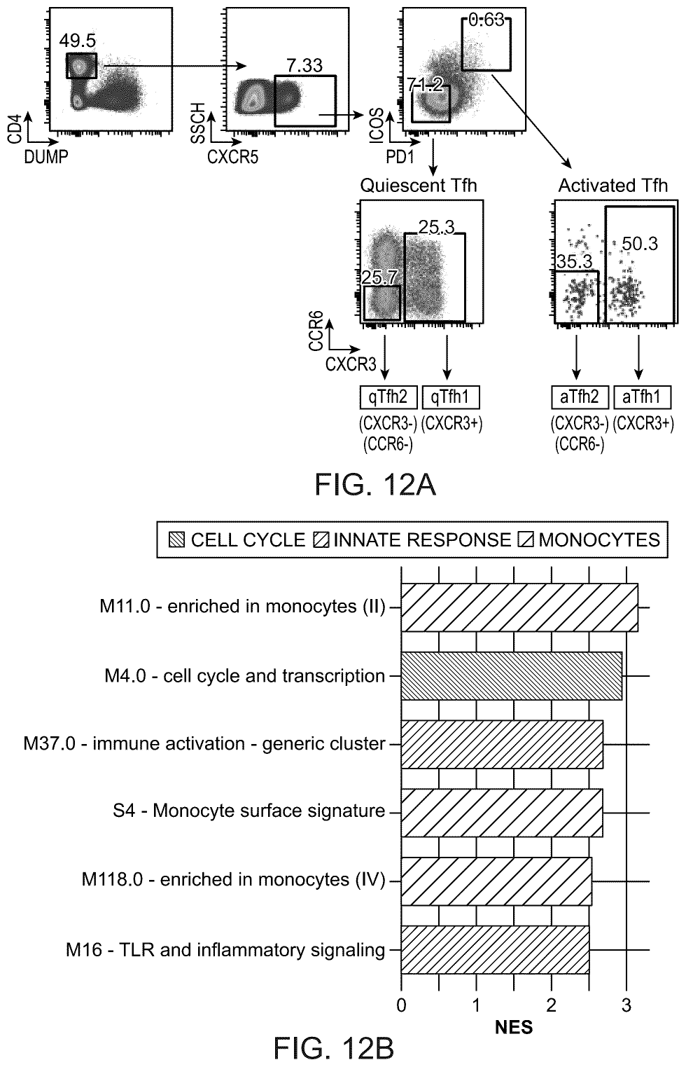

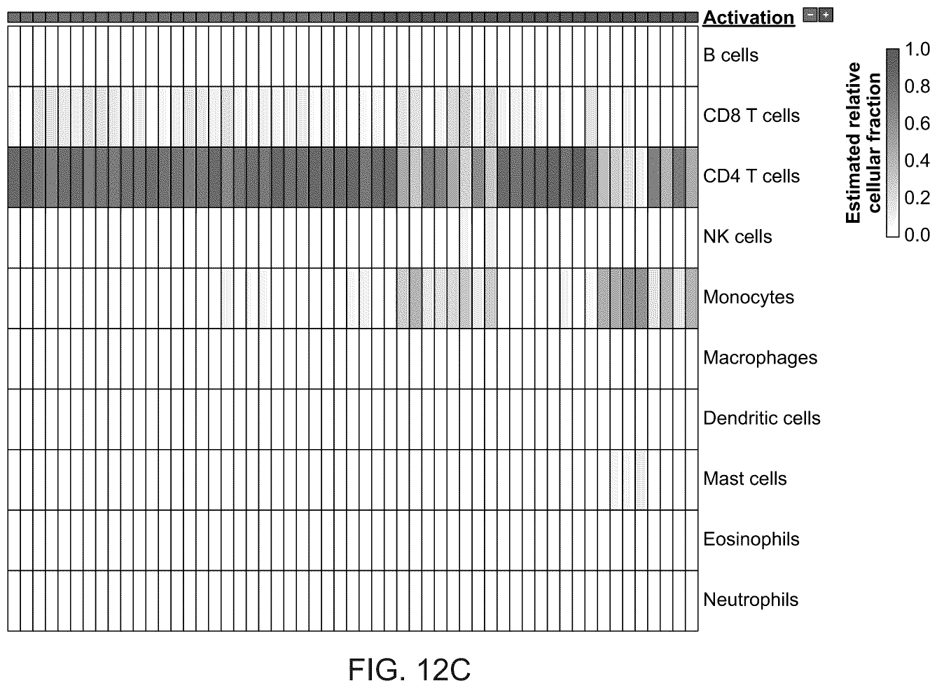

Gene expression analysis of sorted Tfh subsets (as presented in FIG. 12A) revealed significant transcriptional differences between activated versus quiescent Tfh cells, which were consistent in both Tfh1 and Tfh2 populations (FIG. 4D). Activated Tfh upregulated the expression of the FGL2 gene, which encodes fibrinogen-like protein 2, an important immune regulator of both innate and adaptive responses constitutively secreted by both CD4+ and CD8+ T cells (Marazzi et al., 1998), as well as FGFBP2 (previously known as KSP37), whose product is selectively produced by lymphocytes with cytotoxic potential. Surprisingly, we also noticed strong modulation of genes encoding for proteins with known antimicrobial properties typical of innate immune cells, such as lysozyme (LYZ) and IL1B, in addition to C-type lectin receptor CLEC7A, normally found on myeloid cells, and the S100 calcium binding proteins S100A8, S100A9, and S100A12, abundantly present in monocytes. Accordingly, GSEA analysis of DEGs revealed a significant enrichment of monocyte-related BTMs, thus suggesting possible infiltration of other cell types besides blood Tfh in our sorted populations (FIG. 12B). These results were clearly unexpected, since our goal was the selective isolation of peripheral CXCR5+ CD4+ T cell subsets in different activation states. In order to investigate these findings further, we used CIBERSORT, a computational method developed to deconvolve cell type proportions using cell type-specific gene expression references. In agreement with our GSEA analysis, the CIBERSORT algorithm identified a strong enrichment of monocyte-specific genes in the activated Tfh populations (FIG. 12C), thus estimating a significant and selective presence of monocytes among the sorted activated, but not quiescent, blood Tfh cells (FIG. 4E). Recent work described for the first time the existence of CD4+ memory T cell-monocyte complexes in human blood. Notably, these CD3+ CD14+ T cell-monocyte doublets populate the live singlet gate when observed using conventional flow cytometry and form during active immune responses. The origin as well as the biological role of these cellular associations in the peripheral circulation are currently unknown.

Given our initial interest in the transcriptional mechanisms associated with Tfh activation, we used CIBERSORTx to estimate CD4 T cell-specific expression in sorted Tfh cells (hence computationally excluding the monocyte component), and then ran GSEA to identify BTMs enriched in activated versus quiescent Tfh. Not surprisingly, we found strong enrichment in cell cycle- and energy metabolism-related transcriptional modules, indicating a higher metabolic state and increased proliferation for activated Tfh cells versus their quiescent counterpart (FIGS. 4F and 4G). Our analysis also suggests that the activation process of Tfh cells might revolve around polo-like kinase 1 (PLK1), one of the major kinases controlling T cell survival, differentiation, and expansion (FIGS. 4F and 4H). Finally, gene level analysis of sorted Tfh cell populations followed by unsupervised clustering successfully segregated Tfh subsets based on their activation state, highlighting strong upregulation of genes encoding for histone proteins and interferon-stimulated genes (such as ISG15, known for potentiating IFN-γ production) in activated Tfh cells (FIG. 41). No major gene expression clusters that could discriminate between activated Tfh types (Tfh1 versus Tfh2), or activated Tfh at different time-points (d7 versus d28) were observed. Importantly, while AS03+H5N1 induced significantly higher frequencies of circulating activated Tfh after vaccination compared to H5N1 alone, we found no major differences at the transcriptional level between activated Tfh cells isolated from subjects in the two vaccination groups. Taken together, these data suggest that AS03-adjuvanted H5N1 vaccination promotes higher frequencies of circulating, activated Tfh cells after both prime and boost vaccinations compared to unadjuvanted vaccination. Frequencies of activated Tfh cells positively correlated with neutralizing antibodies and antibody affinity to the H5 head domain in the adjuvant group, thus indicating AS03-driven T cell-dependent mechanisms of B cell affinity maturation and antibody production.

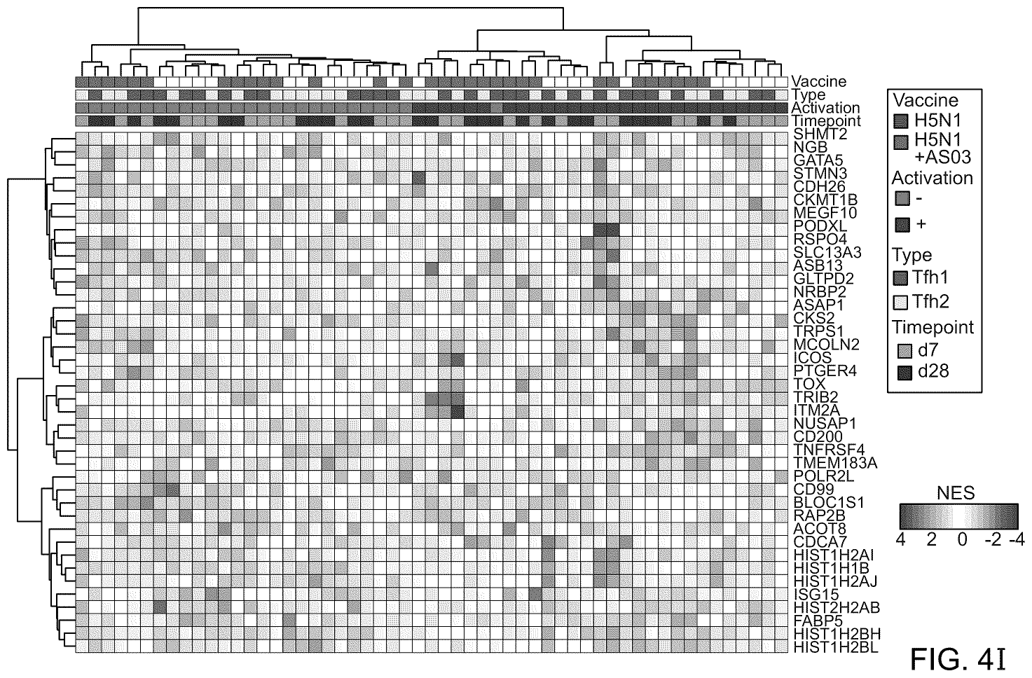

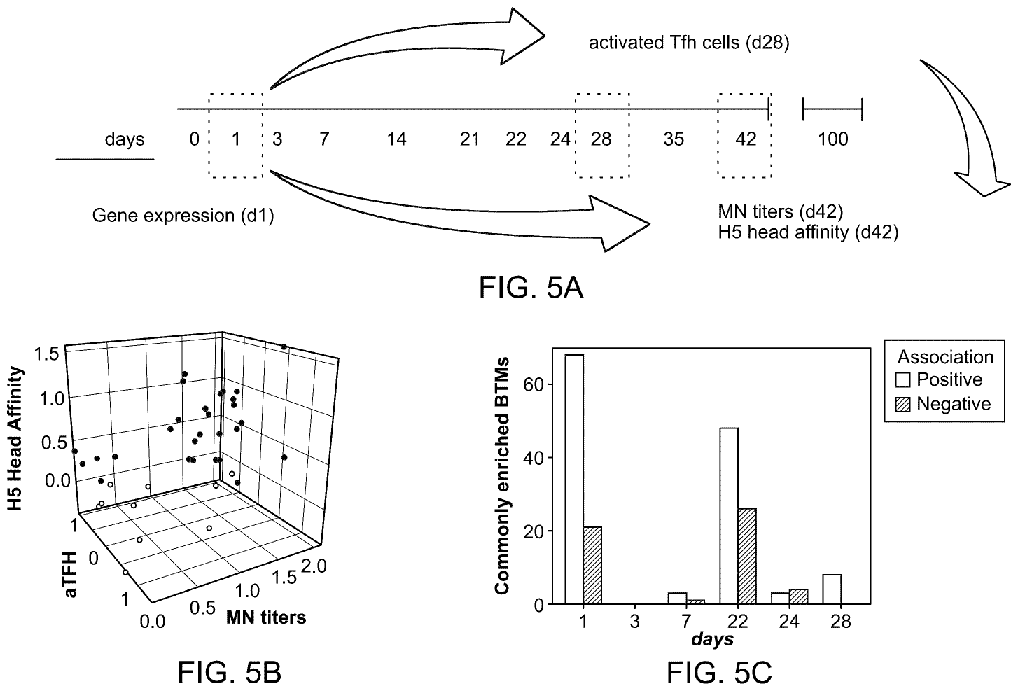

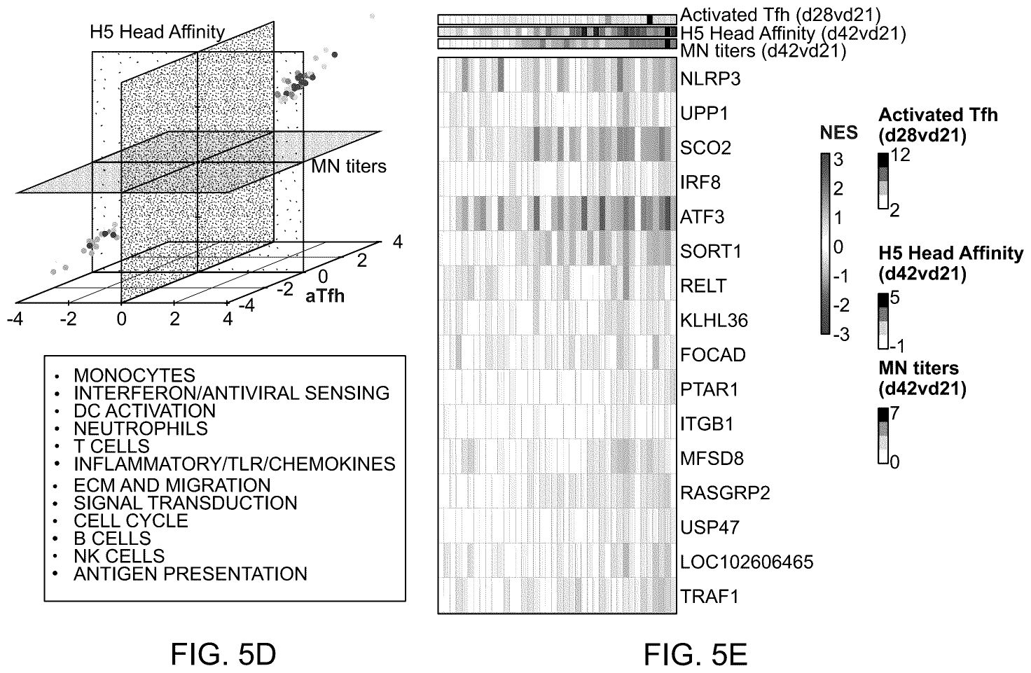

An early molecular signature is associated with multiple markers of post-boost immune response. The strong induction of antibody and Tfh cell responses measured after vaccination in the adjuvant group led us to ask whether it was possible to identify early transcriptional signatures common to the multiple adaptive immunity parameters induced by AS03, specifically increase in i) activated Tfh frequencies; ii) H5 head directed antibody affinity maturation; and iii) neutralizing titers (FIG. 5A). Importantly, these three measurements alone could efficiently segregate subjects by vaccine group, as shown in FIG. 5B, thus implying substantial molecular and cellular differences in the biological mechanisms leading to the generation of adaptive immunity. GSEA performed on genes ranked by correlation with all three parameters revealed that the largest number of commonly enriched modules occurred on day 1 post-prime (FIG. 5C), suggesting that a strong primary response is necessary for the robust adaptive responses induced by AS03 to the boost dose. Many of the commonly enriched modules on day 1 post-prime were associated with inflammatory and interferon signaling, as well as monocyte and DC activation (FIG. 5D). These results are consistent with data from previous systems-based studies with AS03 in clinical settings, where interferon modulation- and monocyte activation-related genes were found to be significantly upregulated within 24 hours after AS03-adjuvanted H1N1 and H5N1 influenza vaccination. Interestingly, strong downregulation in several T and B cell modules on day 1 also correlated with the three highly correlated immune parameters under investigation.

At a gene level, we found that robust day 1 upregulation of the NLRP3 inflammasome gene was strongly associated with both B and T cell responses induced by adjuvanted vaccination with AS03 (FIG. 5E). Alum and squalene-based emulsions have been previously shown to induce NLRP3 inflammasome activation, however the requirement of NRLP3 for these adjuvants to promote adaptive immunity is still under debate. Additionally, we noticed that overexpression of interferon-related genes IRF8 and ATF3 was also positively associated with the potent adaptive immune features promoted by AS03 (FIG. 5E). IRF8 is a transcription and interferon regulatory factor highly expressed in myeloid cells whose function is essential for monocyte and dendritic cell development from their common progenitor. In myeloid cells, IRF8 modulates the expression of Bax and Fas to regulate apoptosis. Interestingly, two papers have recently reported a crucial role for IRF8 in the activation of both NRLC4 and NRLP3 inflammasomes to neutralize bacterial infections in vivo. ATF3, on the other hand, is induced by a wide variety of physiological stresses, and integrates diverse signals stemming from inflammatory events, metabolic stress response, and apoptotic processes. The electron transport chain gene SCO2, which encodes for a metabolic regulator crucial for the generation of ATP and with a role in the prevention of hypoxia-induced cell death, was also highly upregulated after vaccination with AS03 and positively associated with the B and T cell features in analysis. Interestingly, the squalene-based adjuvant MF59, an AS03 analog, has been shown to rely on early ATP production and extracellular release for its mechanisms of adjuvanticity. A potential role for SCO2 in this process at present is unclear. Further, expression of SORT1, which encodes for a protein transporter of the trans-Golgi network that regulates lipid metabolism while also acting as a multi-ligand receptor for inflammatory cytokines including IFN-g and IL-6 in immune cells, also paralleled the increase in the three AS03-induced adaptive immune parameters. Finally, downregulation of TRAF1 on day 1 was negatively associated with increased Tfh activation, antibody-mediated neutralization, and affinity maturation. This gene product acts as a negative regulator of inflammation and, in collaboration with TRAF2 and IAP, mediates anti-apoptotic signals from TNF receptors. Its suppression provides additional evidence that early formation of a pro-inflammatory, pro-apoptotic environment following AS03 injection favors both B and T cell adaptive immune responses to vaccination.

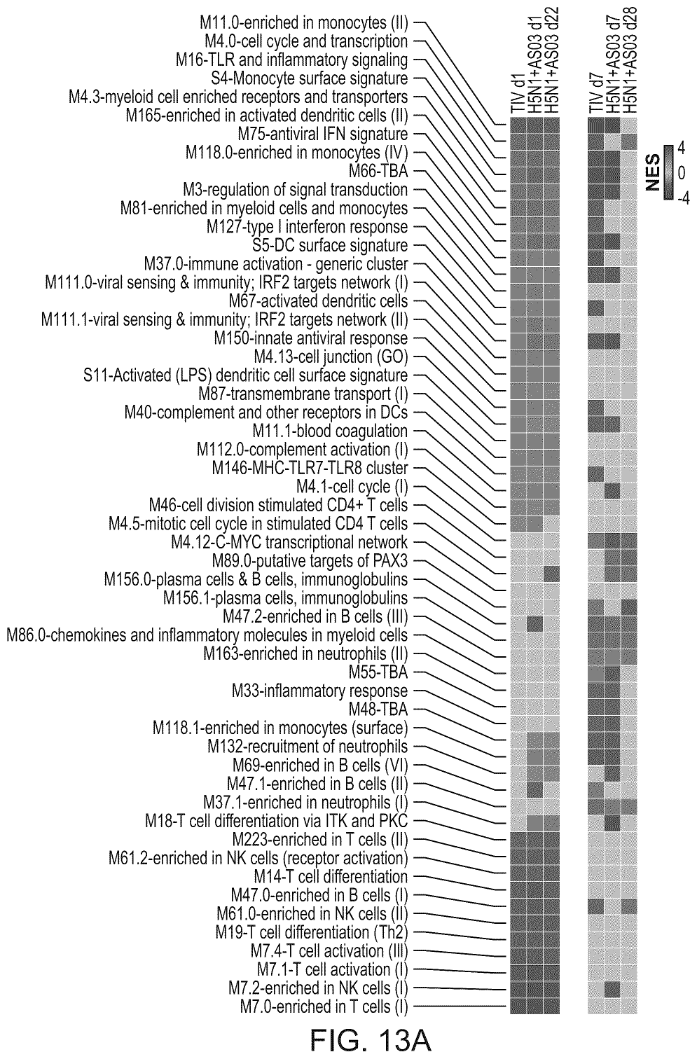

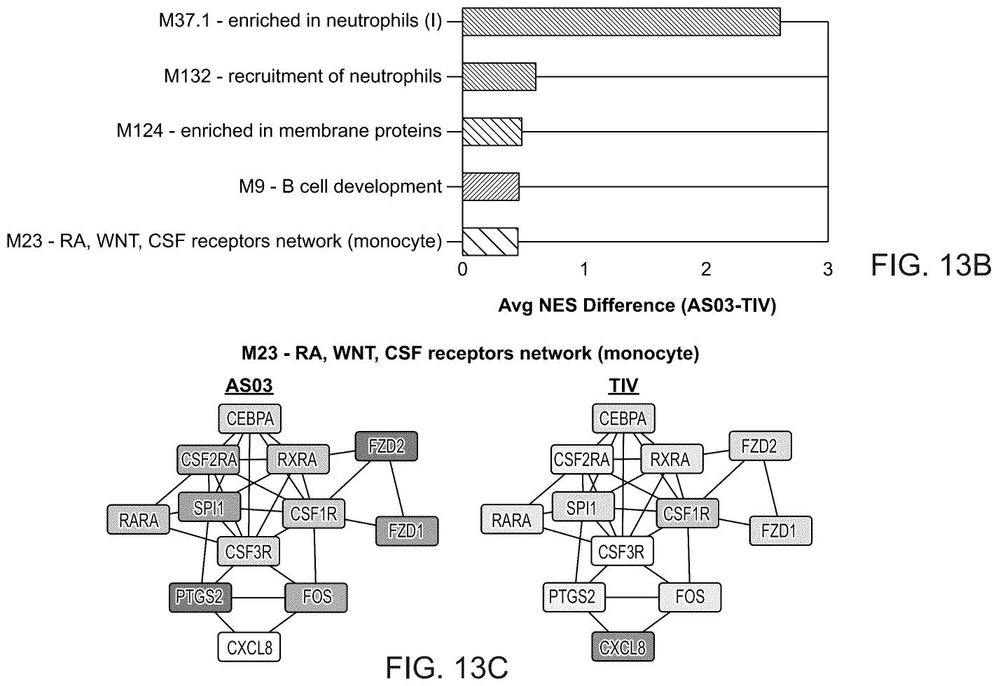

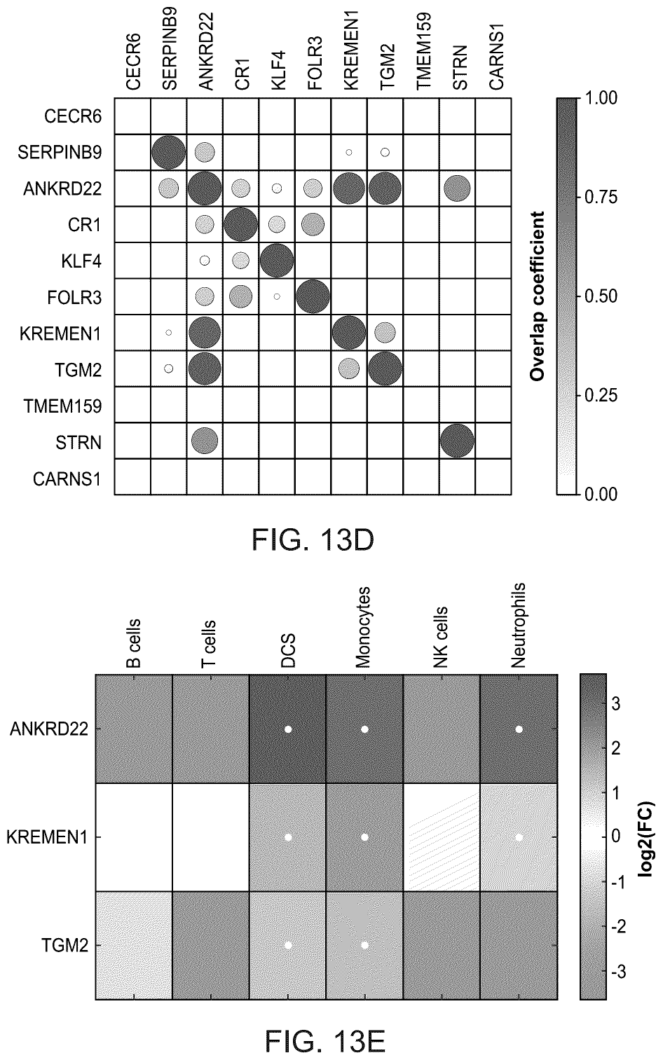

Meta-analysis of influenza vaccine trials reveals an AS03-specific transcriptional signature which correlates with activated Tfh frequencies in the periphery. Although direct comparison between AS03-adjuvanted versus unadjuvanted H5N1 vaccination in this study provided us with valuable insights with regard to the cellular and molecular mechanisms of AS03 adjuvanticity, we sought to extend our investigation to additional influenza datasets available in literature to further dissect the unique role of the AS03 adjuvant in modulating immune responses to vaccination. With this in mind, a key question we wanted to address was the degree to which the immune response to H5N1+AS03 is shared with responses to seasonal influenza strains. To this end, we performed GSEA on genes ranked by post-vaccination fold change on days 1-7 across multiple influenza seasons, using data from our previous work examining responses to trivalent inactivated influenza vaccine (TIV). There was a high degree of overlap among the enriched pathways in response to both vaccines, particularly on days 1 and 7 (FIG. 13A). These results differ from and expand upon earlier findings in this study, where we observed much greater transcriptional differences in both magnitude and kinetics when comparing responses between unadjuvanted vs AS03-adjuvanted H5N1 vaccination. Our data reflect the ability of AS03 to boost naïve immune responses against a flu antigen of avian origin to which humans have otherwise no preexisting immunity, while also promptly elevating the activation status of the immune system to a level comparable to what observed post vaccination with seasonal TIV.

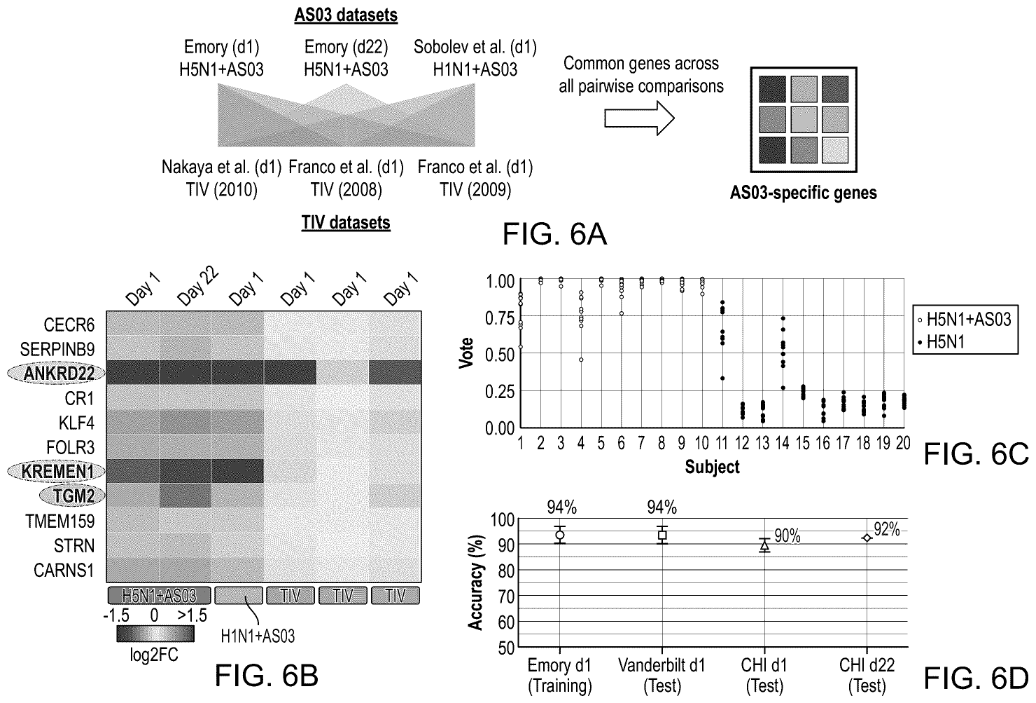

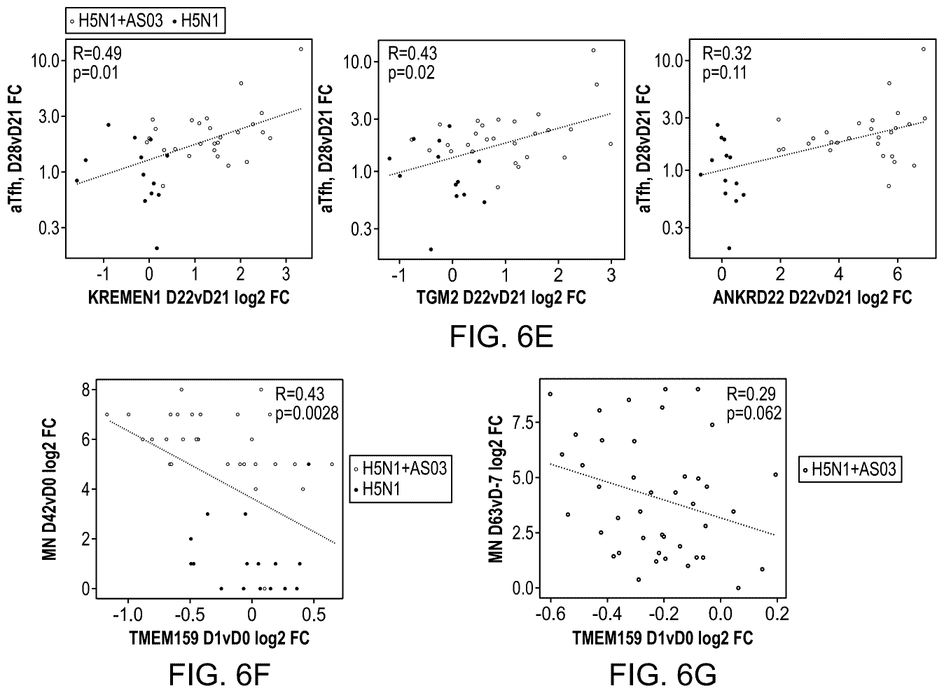

Although responses to H5N1+AS03 and TIV appeared very similar at a broad level, we wondered whether it was possible to identify a transcriptional signature unique to AS03, and therefore not normally present after immunization with seasonal TIV or unadjuvanted H5N1, that may reflect specific mechanisms by which the adjuvant induces a potent immune response. To achieve this goal, we incorporated data from both the prime and boost doses of our trial, as well as publicly available data from a previous study of responses to AS03-adjuvanted H1N1 vaccination, and compared this with gene expression data from multiple TIV trials (FIG. 6A; see also Supplementary Methods). GSEA of genes ranked by average t-statistic between AS03 and seasonal responses revealed multiple neutrophil-related modules, as well as a WNT/retinoic acid receptor (RAR) signaling module (FIG. 13B), that showed significant enrichment in AS03-adjuvanted compared to seasonal responses (FIG. 13C).

We also extended this approach to the gene level by identifying a common set of genes that were differentially expressed in all AS03 datasets when compared to each seasonal dataset in a pairwise fashion (FIG. 6A). We obtained a list of 11 genes (FIG. 6B), many of which, such as ANKRD22, KREMEN1, TGM2, KLF4, TMEM159, STRN are known to be involved in WNT/β-catenin signaling, suggesting a possible role for this pathway in the mechanism of action of AS03. Importantly, to our knowledge none of these 11 genes and their products has been previously linked to the mechanism of action of AS03 or, more generally, has been described to contribute to the generation of immunity to influenza.

To further explore the context for these genes during response to AS03-adjuvanted vaccination, a set of significantly correlating partner genes in all AS03 datasets was determined for each AS03-unique DEG. These partner sets revealed that 3 of the DEGs, ANKRD22, KREMEN1, and TGM2, strongly correlated with each other and shared a large number of co-correlating genes (FIG. 13D). KREMEN1 is known to form a complex with Dickkopf1 (DKK1) and LDL receptor related protein 6 (LRP6) to negatively regulate WNT signaling but the gene has been also further described to work as a dependence receptor that mediates programmed-cell death in a WNT-independent manner by inducing caspase 3 activation. Very little is known about the function of ANKRD22 in immunity, but this gene is highly expressed in several cancerous tissues and its product has been reported to promote cancer progression by favoring metabolic reprogramming of cancer cells. In contrast, the role of TGM2 on the immune system has been better characterized. The gene encodes a multifunctional enzyme belonging to transglutaminases with both pro- and anti-apoptotic roles. In DCs, TGM2 mediates maturation of antigen-presenting cells in response to bacterial LPS and its inhibition significantly reduces cytokine production and DC differentiation. TGM2 activity also plays a crucial role in monocytic differentiation to macrophages and DCs. In agreement with this, deconvolution analysis in our study indicated that strong upregulation of KREMEN1, ANKRD22, and TGM2 1 day following prime and boost vaccinations with AS03 could be traced back to changes in myeloid cell gene expression, particularly DCs and monocytes, but not lymphocytes (FIG. 13E).

In order to confirm the identification of a novel gene signature that could be used as a robust marker of early response to AS03, we used an artificial neural network-based machine learning classification algorithm, trained on expression data of three ‘core’ AS03-specific genes identified from our study, KREMEN1, ANKRD22, and TGM2, to predict vaccine status (adjuvanted vs. unadjuvanted) in three blind test sets containing expression data from independent clinical studies of response to H5N1 vaccination with or without AS03 (see Supplementary Methods). The classifier achieved excellent predictive accuracy (>90%) when tested on gene expression data from sorted monocytes and total PBMCs, thus validating the reproducibility of this AS03-specific signature in external trials (FIGS. 6C and 6D).

Of note, day 1 post-boost expression of KREMEN1, TGM2, and ANKRD22 positively correlated with activated Tfh frequencies at 7 days post-boost (FIG. 6E), possibly suggesting an important role for these genes in the modulation of AS03-induced T cell responses. Interestingly, expression of these three genes was not significantly associated with MN titers (data not shown). These results seem to indicate that AS03 might led to the generation of potent T and B cell responses via multiple, non-overlapping mechanisms. Accordingly, only the expression of one gene in our AS03-specific signature, TMEM159, strongly correlated with MN titers (but not with activated Tfh frequencies) both in our and one other publically available dataset where AS03 was co-administered with an influenza antigen (FIGS. 6F and 6G). Surprisingly, the biological role of the gene product of TMEM159 has been elusive until recently, but elegant work by Chung and colleagues unveiled that this protein, now re-named lipid droplet assembly factor 1 (LDAF1), forms a complex with seipin (encoded by the BSCL2 gene) to determine sites and catalyze lipid droplet formation in the endoplasmic reticulum (ER). In the light of these findings, AS03-induced downregulation of TMEM159 within the first 24 hours after vaccination might indicate the inhibition of novel lipid droplet formation, possibly as a consequence of the accumulation of squalene—a natural precursor of cholesterol which the AS03 adjuvant is mostly composed of—and neutral lipids in the cytosol of immune cells. Although these mechanisms would require further investigation, our results seem to suggest that early AS03-induced biological phenomena underlying TMEM159 downregulation might represent a determining factor for the generation of potent neutralizing antibody responses observed several weeks later (FIGS. 6F and 6G).

AS03 boosts immunogenicity through modulation of immune cell metabolic pathways. The involvement of several of the AS03 ‘core’ genes, including KREMEN1 and TGM2, in biological events related to apoptosis prompted us to explore a potential role for programmed-cell death in the mechanism of action of AS03-adjuvanted vaccines. Contextually, while parsing the transcriptome for early signatures associated to multiple measures of B and T cell immunogenicity, we identified several genes known to be involved in the modulation of cell death and survival, such as IRF8, SCO2, and TRAF1, possibly indicating apoptotic signals as an important factor for the generation of AS03-driven adaptive immunity (FIG. 5).

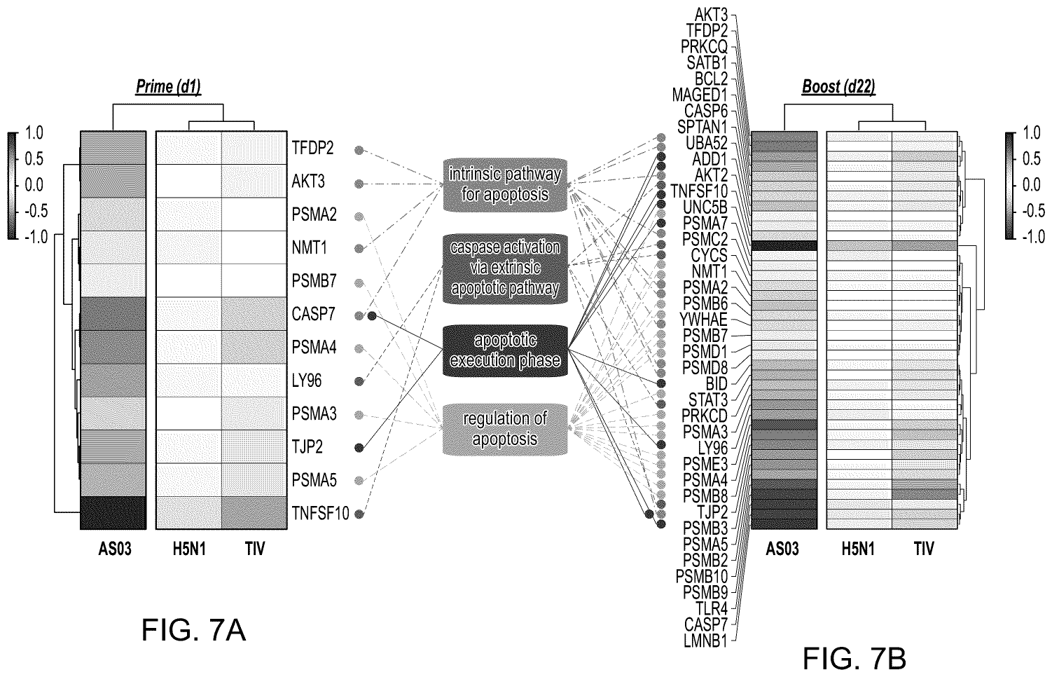

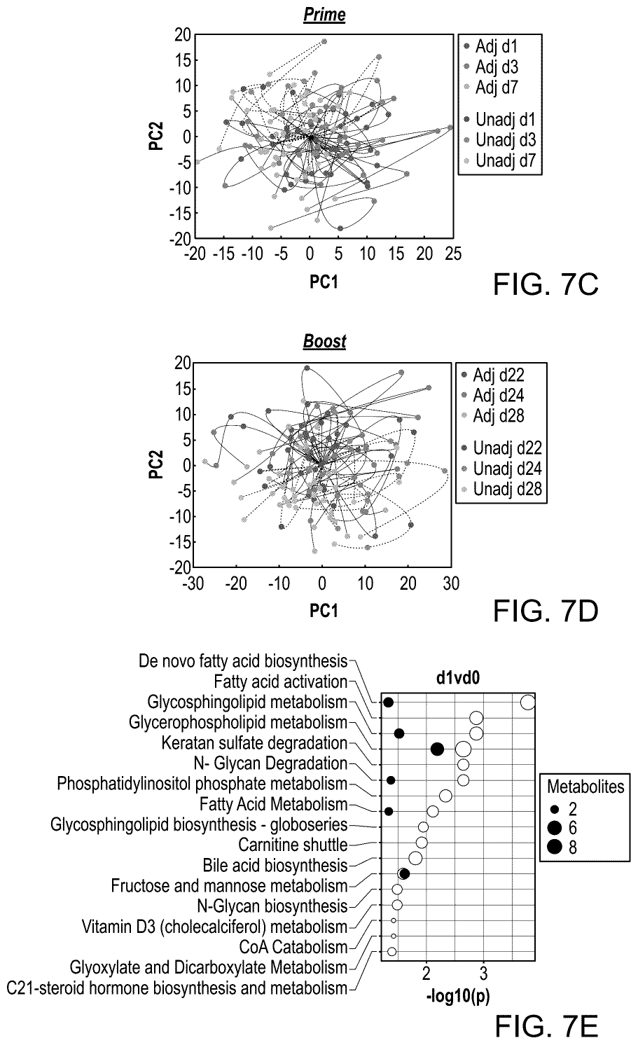

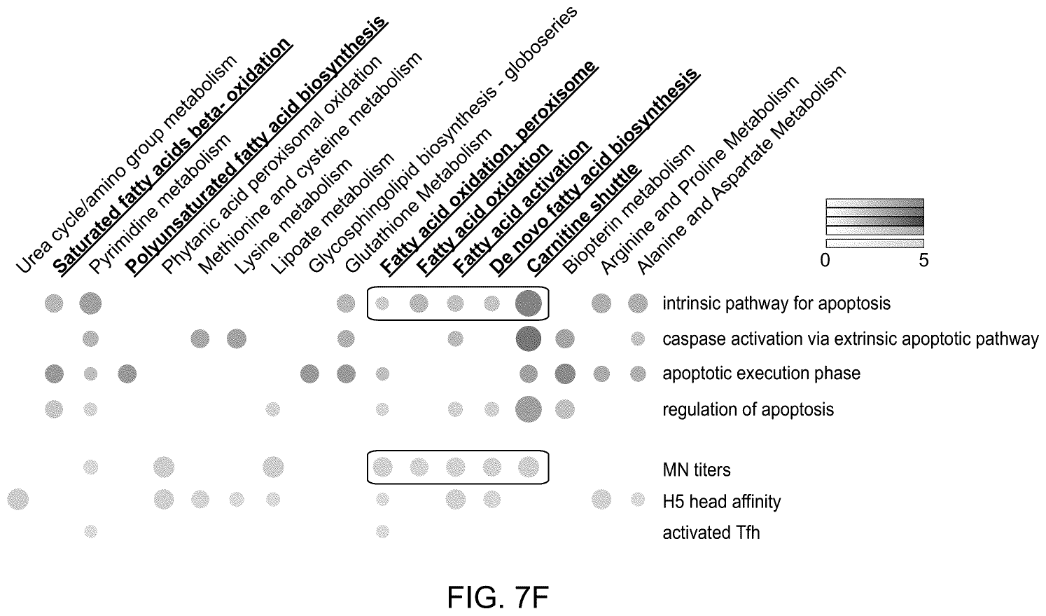

In order to investigate further these mechanisms, we searched the Reactome database for a canonical list of genes involved in different stages of apoptosis and looked for DEGs in the AS03 dataset compared to both unadjuvanted H5N1 or seasonal TIV vaccination in a pairwise fashion. Notably, we discovered significant changes in the expression of 40 apoptosis genes, many of which more strongly regulated on day 1 (day 22) after boost and induced specifically following vaccination with AS03 but not after administration of unadjuvanted vaccines, thus arguing for an intrinsic role of the adjuvant in eliciting mechanisms related to programmed-cell death (FIGS. 7A and 7B). Interestingly, we observed modulation of genes known to participate in both the intrinsic and extrinsic pathways of apoptosis, hinting at the possibility that multiple stimuli, originating inside and outside the cells, might contribute to this process. While transcriptional changes in the extrinsic apoptotic pathway seemed to involve the death receptor TRAIL (TNFSF10) and the TLR4/MD-2 (LY96) axis as indicated by the strong upregulation of these genes, intrinsic mechanisms of apoptosis under mitochondrial control involved downregulation of the genes encoding for the anti-apoptotic molecules BCL-2, AKT2, and AKT3, as well as upregulation of pro-apoptotic molecules BID and caspase activator cytochrome C (CYCS) (FIG. 7B). CASP7, encoding for the executioner caspase 7, was also significantly more upregulated in the AS03+H5N1 group on both day 1 after prime and boost compared to unadjuvanted datasets. In agreement with these findings, we recently reported that immunization with the squalene-based oil-in-water emulsion vaccine adjuvant MF59 induced cell death-associated signaling in lymph node-resident macrophages after MF59 uptake in a murine model of vaccination. Further, here we found that AS03 vaccination promoted a much stronger upregulation of genes encoding for proteasome proteins (PSM-genes), including all three catalytic subunits typical of the immunoproteasome, PSMB8, PSMB9, PSMB10 (also known as LMP7, LMP2, and MECL1, respectively), suggesting adjuvant-driven engagement of a highly efficient proteolytic machinery with distinct immune properties and an established role in the management of oxidative stress.

Previous literature has identified biomarkers of apoptosis as positive predictors of influenza vaccine responsiveness in humans. In light of our findings, we sought to examine the cellular events associated to programmed-cell death signaling arisen following immunization with AS03 that could contribute to immunogenicity. Given the participation of several AS03 ‘core’ genes, such as ANKRD22, TMEM159, KLF4, and TGM2 in key metabolic processes, particularly the control of lipid accumulation and metabolism, we hypothesized that AS03 could promote crucial changes in immune cell metabolism and therefore asked whether we could detect perturbations in the blood metabolome through untargeted high-resolution metabolomics. Indeed, a principal-component analysis (PCA) on fold-change values of differentially abundant metabolite peaks obtained using mummichog software revealed divergence in metabolic trajectories after AS03-adjuvanted vs unadjuvanted H5N1 vaccination, highlighting substantial metabolic differences between vaccine groups, particularly within the first 24 hours after each immunization (FIGS. 7C and 7D). Enrichment analysis of differential features unveiled AS03-induced changes in multiple pathways related to lipid metabolism and fatty acid metabolism and activation, as illustrated in FIG. 7E. Importantly, we noticed significant correlations between the gene expression changes in apoptotic pathways and abundance of specific metabolites (FIG. 7F). Of special interest, we found strong associations between apoptosis-related genes and metabolic perturbations in the fatty acid and carnitine shuttle pathways. The carnitine shuttle represents a system by which long-chain fatty acids, which are impermeable to the mitochondrial membrane, are transported into the mitochondrial matrix to undergo β-oxidation and generate energy under the form of acetyl-CoA (which will then enter the citric acid cycle). Consistently, significant correlations between other metabolic pathways related to β-oxidation of saturated fatty acids and apoptosis gene signatures were also identified, including peroxisomal oxidation, indicating that AS03 might induce rapid and strong changes in cellular fatty acid metabolism within the first 24 hours after vaccination. Being fatty acid oxidation known to be one of the main causes for the generation of reactive oxygen species (ROS), we hypothesize that excessive ROS production as a consequence of increased fatty acid oxidation might represent one of the main signals by which the AS03 adjuvant triggers immunogenic mechanisms of programmed-cell death. In agreement with this hypothesis, AS03-induced metabolic changes in fatty acid activation and metabolism were strongly associated with the generation of vaccine-specific neutralizing antibodies (FIG. 7F).

Here we presented a detailed multi-omics analysis of cellular, transcriptional, and metabolic responses to a prepandemic H5N1 avian influenza vaccine administered with and without the squalene-based emulsion adjuvant AS03 in a cohort of healthy volunteers. By extending our analyses to several other AS03-adjuvanted and unadjuvanted influenza study datasets deposited in public repositories, we were able to greatly expand upon previous systems biological reports on the use of AS03 in humans and identified several gene signatures, metabolic networks, and biological processes, unappreciated to date, whose distinct modulation within the first few days following vaccination with the AS03 adjuvant could be linked to, and in some cases predict, one or multiple measures of vaccine immunogenicity in this and independent clinical studies.

Among the initial findings in this study are the transcriptional differences, both quantitative and qualitative, observed in the innate immune responses following prime and boost immunization with AS03. As an example, this was the case of genes orchestrated by the transcriptional factor PAX3 encoding for important chemoattractants and modulators of innate immune cells, whose upregulation was more pronounced and longer sustained after boost compared to the same time-point after prime immunization. The relatively novel concept of ‘innate immune memory’ (or ‘trained immunity’), a phenomenon by which innate immune cells, such as monocytes, macrophages, or NK cells, can temporarily ‘remember’ previous exposure to endogenous or exogenous stimuli via epigenetic modifications thus altering their behavior to subsequent immunizations, has been only marginally explored in the context of AS03-adjuvanted vaccination strategies. Previous clinical studies, however, have shown how similar mechanisms might apply to other adjuvants, such AS01 and AS02, and antigens. In this context, with several COVID-19 vaccine technology platforms in late stage of development incorporating adjuvants and requiring multiple immunizations to induce and sustain protection over time, there is an unprecedented opportunity to systematically explore and define the effects of trained immunity-related phenomena on vaccination outcomes on a global scale. These studies could be of strategic importance to inform the clinical practice and identify optimal homologous or heterologous prime-boost vaccination regimens, thus enabling more efficient global vaccination campaigns.

Whereas immune responses to natural infection with influenza virus in humans are relatively broad and long-lived, vaccine-induced immunity mostly induces systemic antibody responses that tend to wane over time. A major goal of our study was the investigation of the molecular mechanisms that underlie vaccine-induced durable antibody responses. Here we identified a blood transcriptional signature of cellular migration associated with a more persistent antibody response to AS03-adjuvanted H5N1 vaccination that we later used to successfully predict, in a blind fashion, antibody durability in an independent clinical study using the same vaccine. Notably, CITE-seq experiments identified platelets as the cellular origin of this longevity signature, thus indicating these cells as potential players in the formation of long-lived antibody responses to vaccination. Previous work in mice has demonstrated that bone marrow-resident megakaryocytes, the platelet precursors, constitute a functional component of the microenvironmental niches that are crucial for the generation and maintenance of long-lived plasma cells by interacting and producing the plasma cell survival factors APRIL and IL-6. Whether similar mechanisms might be involved in the generation of long-lived antibody responses to vaccination in humans is unclear at present. Further, CITE-seq analysis revealed that waning antibody responders showed a much sharper decrease in platelet RNA content after the second vaccination compared to more persistent responders. Platelets lack genomic DNA and the ability to synthesize new mRNA; however, they inherit mRNA and ribosomes from the precursor bone marrow-resident megakaryocytes when newly released in the peripheral circulation. The overall decrease in platelet RNA content observed in waning antibody responders might therefore reflect a process of cellular maturation and aging, with progressive loss of the initial megakaryocytic features in favor of a more mature platelet phenotype. Platelets, however, have also the ability to horizontally transfer RNA to other cells, such as monocytes and endothelial cells, and subsequently alter the expression profile of recipient cells to regulate inflammation and vascular homeostasis. Further research is needed to clarify the mechanisms by which platelets and megakaryocytes contribute to long-lasting antibody responses in humans.

Finally, by performing a meta-analysis of AS03-adjuvanted and unadjuvanted influenza vaccine datasets, we were able to identify a common set of genes induced specifically by AS03 which were not previously linked to the adjuvant's mechanism of action or known to contribute to the generation of immunity to influenza. By using the day 1 changes in expression of three of these genes, TGM2, ANKRD22, and KREMEN1, we could predict adjuvant use in external trials with more than 90% accuracy. Remarkably, early transcriptional changes in the AS03 ‘core’ genes were strongly associated with frequencies of activated Tfh cells in the periphery 7 days after vaccination, suggesting a possible participation for these genes in mechanisms of immunogenicity. Contextually, pathway analysis supported a critical role for intrinsic (mitochondrial) and extrinsic pathways of apoptosis in the mode of action of AS03. In agreement with this, we previously found that immunization with the squalene-based emulsion adjuvant MF59 induced apoptotic signals in lymph node-resident macrophages after adjuvant uptake in mice. Importantly, in vivo co-administration of pan-caspase inhibitors and MF59 significantly dampened the production of IgG antibody responses enhanced by the adjuvant, underscoring a crucial role for apoptosis and caspases in the mechanism of action of squalene emulsion adjuvants. Intrinsic stresses, such as DNA damage, hypoxia, excessive production of ROS, or metabolic dysfunctions have all been established as potential causes of mitochondrial apoptosis. Accordingly, plasma metabolomics analysis revealed that AS03-induced early perturbations in lipid and fatty acid oxidation and metabolism were highly associated with expression of the AS03-induced genes involved in mitochondrial apoptosis, as well as with neutralizing antibody titers several weeks later. Of note, we already found fatty acid metabolism to be an important orchestrator of antibody responses to influenza vaccination. Overall, our results are also consistent with earlier work in mice with AS03, where gene signatures of alteration in lipid metabolism could be detected in draining LNs of immunized mice within 2 hours after vaccination. Similarly, it was previously shown that in vitro uptake of other squalene containing-emulsion adjuvants by phagocytic and non-phagocytic cells led to lipid alterations and accumulation of neutral lipids in the form of cytoplasmic lipid droplets.

In conclusion, our findings revealed previously unappreciated biological mechanisms associated with AS03 adjuvanticity and antibody durability following prepandemic H5N1 vaccination in humans, thus underscoring once more the enormous potential of systems biological approaches in accelerating vaccine research and development.

### Example 2

### Use of Platelets to Prognose Durability of Immune Response

### Results

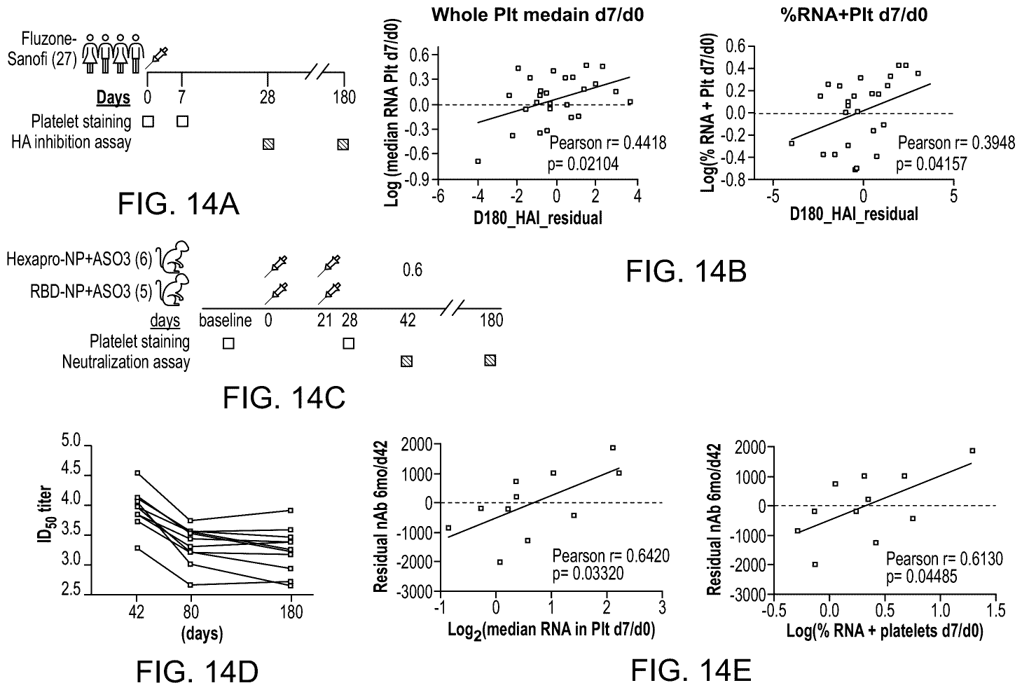

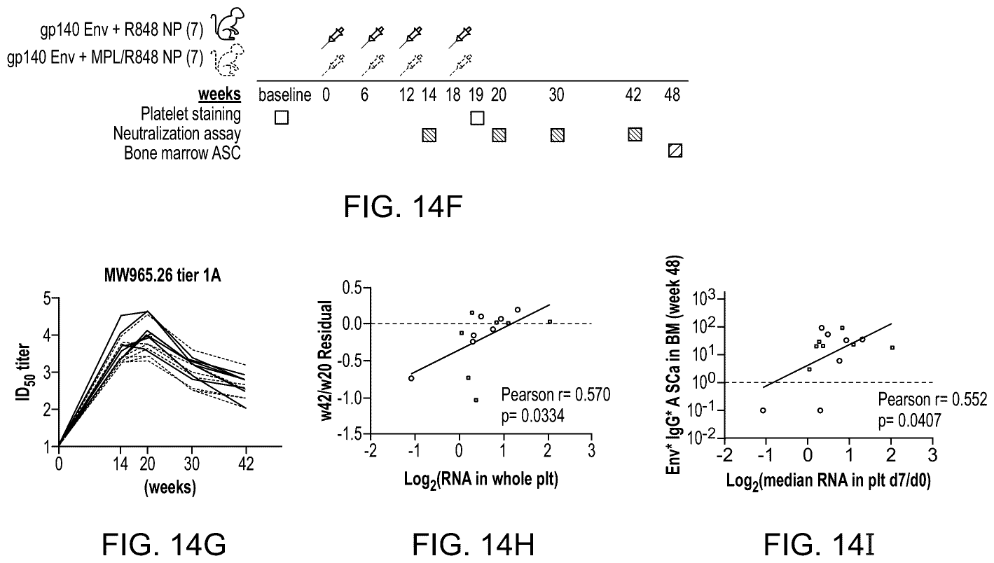

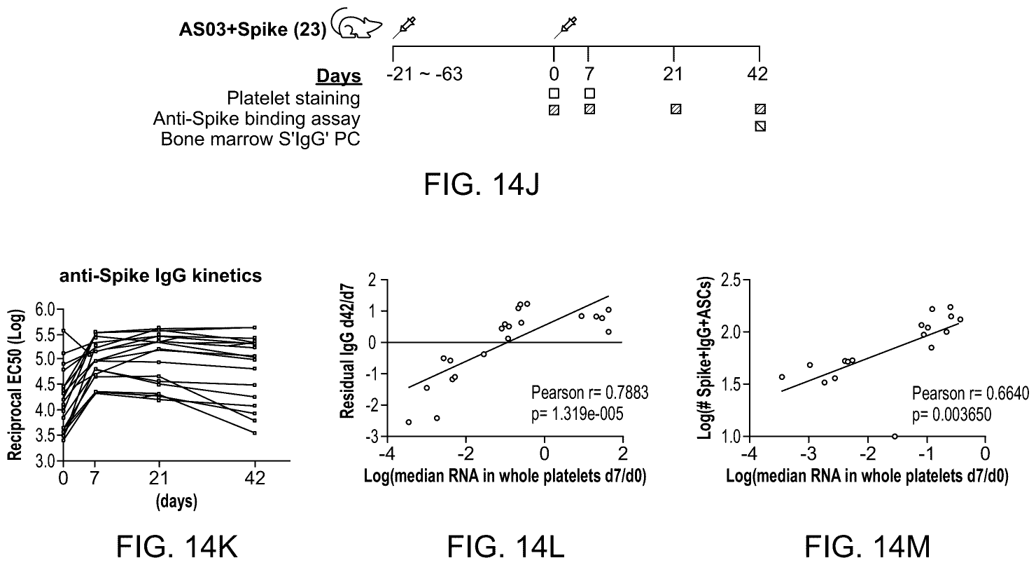

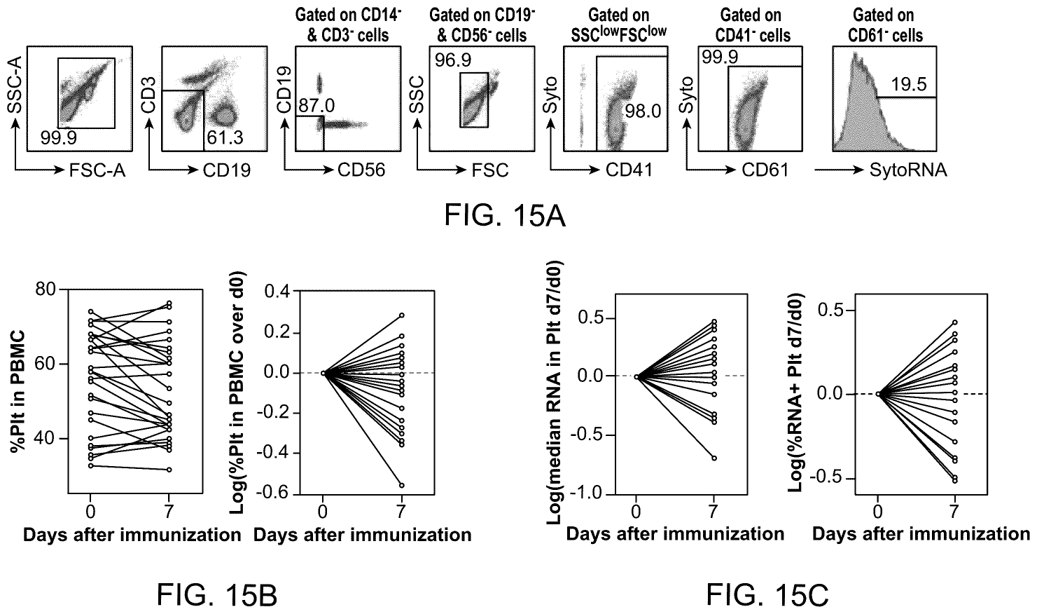

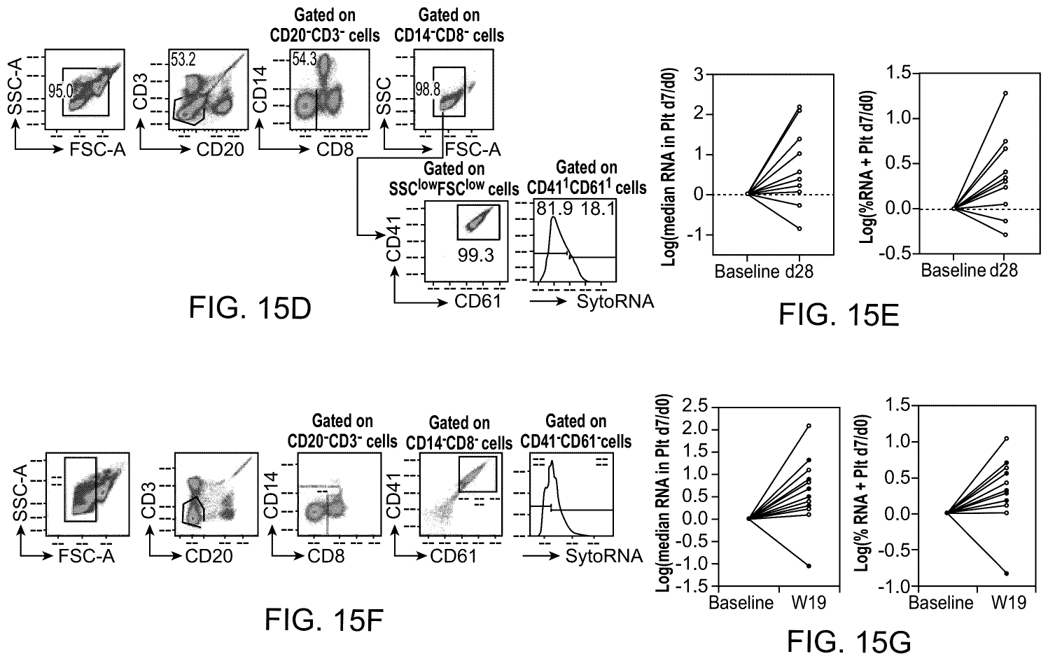

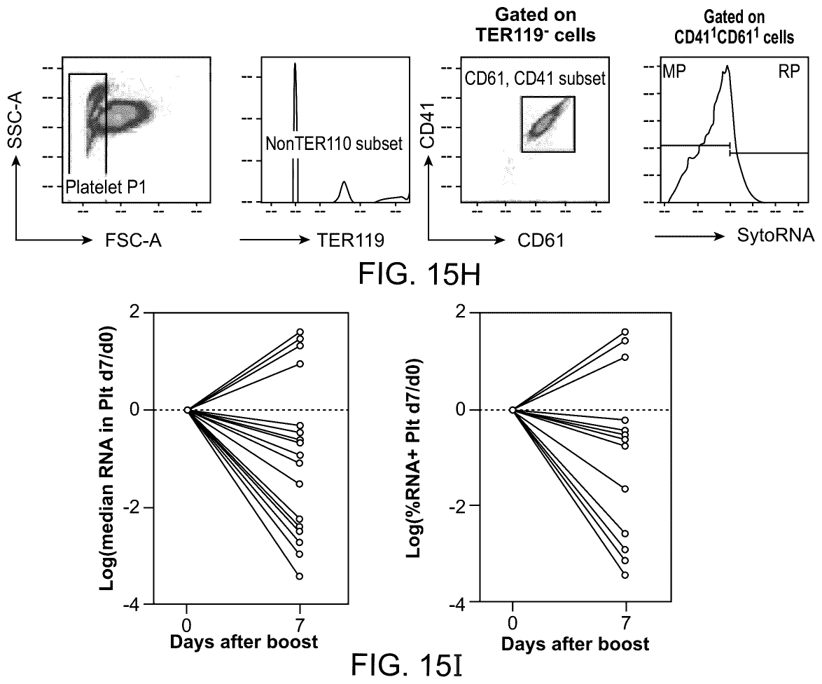

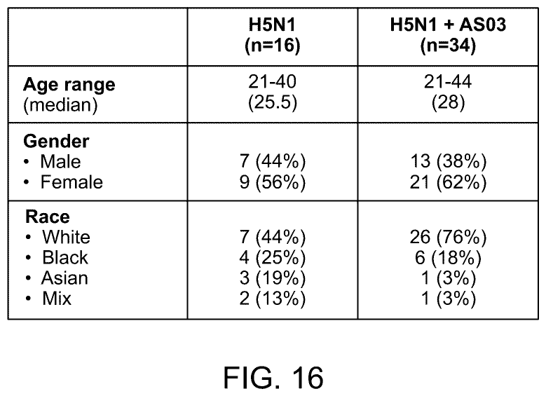

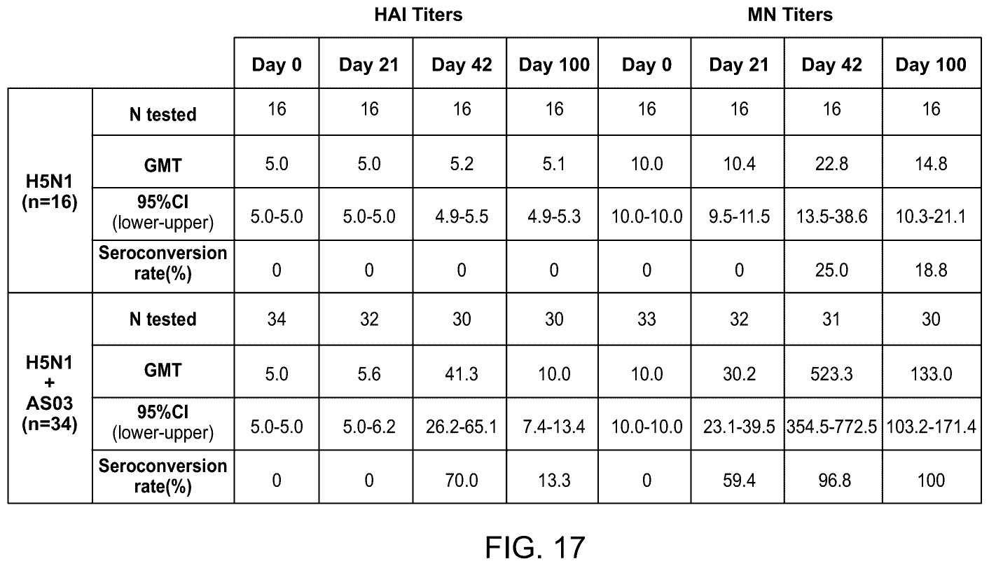

Platelet RNA content is positively correlated with persistence of antibody responses to vaccination. To validate our CITE-seq findings (FIG. 3), we used flow cytometric analysis to assess the RNA content in platelets from subjects immunized with TIV during the 2010-11 influenza season (FIG. 14A). FSC/SSC dot plots of thawed PBMC samples showed CD41⁺CD61⁺ platelet populations, which are characterized by low SSC and FSC values and can be easily distinguished from PBMCs by size (FIG. 15A). Platelet frequencies in these samples were comparable to whole PBMCs and did not differ between days 0 and 7 post TIV vaccination (FIG. 15B), supporting the notion that the platelet signature was arising due to intrinsic differences within platelets rather than differing platelet numbers. Importantly, the fold change of RNA content in whole platelets and % RNA⁺ platelets at day 7 versus day 0 in vaccinees (FIG. 15C) positively correlated to the day 180/day 42 residual (FIG. 14B), confirming the association between platelet RNA content and persistence of antibody responses to vaccination.

To explore the mechanisms underlying this association in more detail, we next examined responses to AS03-adjuvanted SARS-COV-2 subunit Spike protein in Rhesus macaques (FIG. 14C). As with the human samples, the FSC/SSC dot plot of the NHP samples also showed significant platelet counts (FIG. 15D). Neutralizing antibody responses peaked at day 42 and then gradually declined (FIG. 14D). The fold changes of RNA content in whole platelets and % RNA⁺ platelets at day 7 versus baseline were correlated with the day 180/day 42 residual (FIG. 14E and FIG. 15E). Consistently, we observed a positive correlation between the fold change of platelet RNA content at day 7 and the week 42/week 20 residual in NHP subjects vaccinated with R848-adjuvanted HIV subunit gp140 (FIGS. 14F-H and FIGS. 15F, 1G). As long-term antibody titers are predominately generated by long-lived plasma cells (LLPCs) that reside in the bone marrow, the frequency of bone marrow antigen-specific plasma cells can be an indicator of durable neutralizing antibody response (Kasturi et al, 2015, science immunology). Our data showed that the fold change of platelet RNA content at day 7 was also associated with the number of bone marrow ASCs at week 48 (FIG. 141). To support our findings in humans and NHPs, we used mice vaccinated with AS03-adjuvanted SARS-COV2 subunit Spike to evaluate platelet RNA content (FIGS. 14J, 14K and FIGS. 15H, 15I) and found that the fold change of platelet RNA content at day 7 was correlated with the day 42/day 7 residual or the number of ASC at day 42 post boost (FIGS. 14L, 14K).

Previous work in mice has demonstrated that bone marrow-resident megakaryocytes, the platelet precursors, constitute a functional component of the microenvironmental niches that are crucial for the generation and maintenance of long-lived plasma cells (LLPCs) by interacting and producing the plasma cell survival factors APRIL and IL-6 (Winter et al, 2010, Blood). Our results demonstrate that peripheral platelet RNA content can reflect the status of megakaryocytes and other processes in the bone marrow contributing to LLPC survival and point to platelet RNA content as a biomarker for predicting the durability of antibody responses to vaccines.

### Methods

Previously cryopreserved PBMCs from human and Rhesus monkeys were thawed, washed in PBS 1×, and stained in 100 μl of PBS containing 1.5 UM SYTO™ RNASelect™ Green Fluorescent cell Stain (S32703, Invitrogen) at room temperature with an appropriate antibody cocktail. Twenty min later, 300 μl of 1% paraformaldehyde was directly added to samples. Cells were analyzed on a FACS Symphony flow cytometer (BD Biosciences) on the same day. The threshold for the FSC value was set to 4000 to ensure visualization of platelet population. Analysis of flow cytometry files was performed using the FlowJo software (FlowJo, LLC). For the identification of PBMC-free platelets in thawed human PBMCs, a cocktail of anti-CD3-BUV737, anti-CD19-APC, anti-CD14-BV605, anti-CD56-PE, and anti-CD41-BV421, anti-CD61-PE-Cy7 antibodies was used. Platelets were defined as CD41⁺CD61⁺ cells after the exclusion of CD3⁺, CD19⁺, CD14⁺ and CD56⁺ cells. For the identification of PBMC-free platelets in thawed NHP PBMCs, a cocktail of anti-CD3-PE-CF594, anti-CD8-BUV563, anti-CD20-BUV737, anti-CD14-BUV805, and anti-CD41-BV421, anti-CD61-PE-Cy7 antibodies was used. Platelets were defined as CD41⁺CD61⁺ cells after the exclusion of CD3⁺, CD8⁺, CD20⁺ and CD14⁺ cells.

Mouse blood anticoagulated with citrate-dextrose solution (sc-214744, Santa Cruz Biotechnology, Inc.) at a ratio of 6-8:1 was centrifugated at 150/g for 10 min at room temperature to obtain platelet-rich plasma. Twenty μl of freshly prepared platelet-rich plasma was mixed with 80 μl of PBS containing 1.5 M RNASelect™ Stain for 20 min at room temperature with 0.6 μl of anti-TER119-PE, anti-CD41-BV421, and anti-CD61-PE-Cy7 antibodies. Mouse platelets were defined as CD41⁺CD61⁺ cells after the exclusion of TER119⁺ red blood cells.

### REFERENCES

Arunachalam, P. S., Wimmers, F., Mok, C. K. P., Perera, R., Scott, M., Hagan, T., Sigal, N., Feng, Y., Bristow, L., Tak-Yin Tsang, O., et al. (2020). Systems biological assessment of immunity to mild versus severe COVID-19 infection in humans. Science 369, 1210-1220.
Belongia, E. A., Sundaram, M. E., McClure, D. L., Meece, J. K., Ferdinands, J., and VanWormer, J. J. (2015). Waning vaccine protection against influenza A (H3N2) illness in children and older adults during a single season. Vaccine 33, 246-251.
Birsoy, K., Chen, Z., and Friedman, J. (2008). Transcriptional regulation of adipogenesis by KLF4. Cell Metab 7, 339-347.
Burel, J. G., Pomaznoy, M., Lindestam Arlehamn, C. S., Weiskopf, D., da Silva Antunes, R., Jung, Y., Babor, M., Schulten, V., Seumois, G., Greenbaum, J. A., et al. (2019). Circulating T cell-monocyte complexes are markers of immune perturbations. Elife 8.
Burny, W., Callegaro, A., Bechtold, V., Clement, F., Delhaye, S., Fissette, L., Janssens, M., Leroux-Roels, G., Marchant, A., van den Berg, R. A., et al. (2017). Different Adjuvants Induce Common Innate Pathways That Are Associated with Enhanced Adaptive Responses against a Model Antigen in Humans. Front Immunol 8, 943.
Causeret, F., Sumia, I., and Pierani, A. (2016). Kremen1 and Dickkopf1 control cell survival in a Wnt-independent manner. Cell Death Differ 23, 323-332.
Chevalier, N., Jarrossay, D., Ho, E., Avery, D. T., Ma, C. S., Yu, D., Sallusto, F., Tangye, S. G., and Mackay, C. R. (2011). CXCR5 expressing human central memory CD4 T cells and their relevance for humoral immune responses. J Immunol 186, 5556-5568.
Chu, D. W., Hwang, S. J., Lim, F. S., Oh, H. M., Thongcharoen, P., Yang, P. C., Bock, H. L., Drame, M., Gillard, P., Hutagalung, Y., et al. (2009). Immunogenicity and tolerability of an AS03 (A)-adjuvanted prepandemic influenza vaccine: a phase III study in a large population of Asian adults. Vaccine 27, 7428-7435.
Chung, J., Wu, X., Lambert, T. J., Lai, Z. W., Walther, T. C., and Farese, R. V., Jr. (2019). LDAF1 and Seipin Form a Lipid Droplet Assembly Complex. Dev Cell 51, 551-563 e557.
Cortese, M., Sherman, A. C., Rouphael, N. G., and Pulendran, B. (2020). Systems Biological Analysis of Immune Response to Influenza Vaccination. Cold Spring Harb Perspect Med.
Couch, R. B., Bayas, J. M., Caso, C., Mbawuike, I. N., Lopez, C. N., Claeys, C., El Idrissi, M., Herve, C., Laupeze, B., Oostvogels, L., et al. (2014). Superior antigen-specific CD4+ T-cell response with AS03-adjuvantation of a trivalent influenza vaccine in a randomised trial of adults aged 65 and older. BMC Infect Dis 14, 425.
Crotty, S. (2011). Follicular helper CD4 T cells (TFH). Annu Rev Immunol 29, 621-663.
Crotty, S., Felgner, P., Davies, H., Glidewell, J., Villarreal, L., and Ahmed, R. (2003). Cutting edge: long-term B cell memory in humans after smallpox vaccination. J Immunol 171, 4969-4973.
Dai, F., Lee, H., Zhang, Y., Zhuang, L., Yao, H., Xi, Y., Xiao, Z. D., You, M. J., Li, W., Su, X., et al. (2017). BAP1 inhibits the ER stress gene regulatory network and modulates metabolic stress response. Proc Natl Acad Sci USA 114, 3192-3197.
Eckert, R. L., Kaartinen, M. T., Nurminskaya, M., Belkin, A. M., Colak, G., Johnson, G. V., and Mehta, K. (2014). Transglutaminase regulation of cell function. Physiol Rev 94, 383-417.
Eisenbarth, S. C., Colegio, O. R., O'Connor, W., Sutterwala, F. S., and Flavell, R. A. (2008). Crucial role for the Nalp3 inflammasome in the immunostimulatory properties of aluminium adjuvants. Nature 453, 1122-1126.
Ellebedy, A. H., Nachbagauer, R., Jackson, K. J. L., Dai, Y. N., Han, J., Alsoussi, W. B., Davis, C. W., Stadlbauer, D., Rouphael, N., Chromikova, V., et al. (2020). Adjuvanted H5N1 influenza vaccine enhances both cross-reactive memory B cell and strain-specific naïve B cell responses in humans. Proc Natl Acad Sci USA 117, 17957-17964.
Evans, P. M., Zhang, W., Chen, X., Yang, J., Bhakat, K. K., and Liu, C. (2007). Kruppel-like factor 4 is acetylated by p300 and regulates gene transcription via modulation of histone acetylation. J Biol Chem 282, 33994-34002.
Ferdinands, J. M., Fry, A. M., Reynolds, S., Petrie, J., Flannery, B., Jackson, M. L., and Belongia, E. A. (2017). Intraseason waning of influenza vaccine protection: Evidence from the US Influenza Vaccine Effectiveness Network, 2011-12 through 2014-15. Clin Infect Dis 64, 544-550.
Franchi, L., and Nunez, G. (2008). The Nlrp3 inflammasome is critical for aluminium hydroxide-mediated IL-1beta secretion but dispensable for adjuvant activity. Eur J Immunol 38, 2085-2089.
Franco, L. M., Bucasas, K. L., Wells, J. M., Nino, D., Wang, X., Zapata, G. E., Arden, N., Renwick, A., Yu, P., Quarles, J. M., et al. (2013). Integrative genomic analysis of the human immune response to influenza vaccination. Elife 2, e00299.
Furman, D., Jojic, V., Kidd, B., Shen-Orr, S., Price, J., Jarrell, J., Tse, T., Huang, H., Lund, P., Maecker, H. T., et al. (2013). Apoptosis and other immune biomarkers predict influenza vaccine responsiveness. Mol Syst Biol 9, 659.
Garcon, N., Vaughn, D. W., and Didierlaurent, A. M. (2012). Development and evaluation of AS03, an Adjuvant System containing alpha-tocopherol and squalene in an oil-in-water emulsion. Expert Rev Vaccines 11, 349-366.
Gilchrist, M., Thorsson, V., Li, B., Rust, A. G., Korb, M., Roach, J. C., Kennedy, K., Hai, T., Bolouri, H., and Aderem, A. (2006). Systems biology approaches identify ATF3 as a negative regulator of Toll-like receptor 4. Nature 441, 173-178.
Givord, C., Welsby, I., Detienne, S., Thomas, S., Assabban, A., Lima Silva, V., Molle, C., Gineste, R., Vermeersch, M., Perez-Morga, D., et al. (2018). Activation of the endoplasmic reticulum stress sensor IRE1alpha by the vaccine adjuvant AS03 contributes to its immunostimulatory properties. NPJ Vaccines 3, 20.
Grigoryan, L., and Pulendran, B. (2020). The immunology of SARS-COV-2 infections and vaccines. Semin Immunol 50, 101422.
Hagan, T., Cortese, M., Rouphael, N., Boudreau, C., Linde, C., Maddur, M. S., Das, J., Wang, H., Guthmiller, J., Zheng, N.Y., et al. (2019). Antibiotics-Driven Gut Microbiome Perturbation Alters Immunity to Vaccines in Humans. Cell 178, 1313-1328 e1313.
Hagan, T., and Pulendran, B. (2018). Will Systems Biology Deliver Its Promise and Contribute to the Development of New or Improved Vaccines? From Data to Understanding through Systems Biology. Cold Spring Harb Perspect Biol 10.
Hammarlund, E., Lewis, M. W., Hansen, S. G., Strelow, L. I., Nelson, J. A., Sexton, G. J., Hanifin, J. M., and Slifka, M. K. (2003). Duration of antiviral immunity after smallpox vaccination. Nat Med 9, 1131-1137.
Hartman, M. G., Lu, D., Kim, M. L., Kociba, G. J., Shukri, T., Buteau, J., Wang, X., Frankel, W. L., Guttridge, D., Prentki, M., et al. (2004). Role for activating transcription factor 3 in stress-induced beta-cell apoptosis. Mol Cell Biol 24, 5721-5732.
He, J., Tsai, L. M., Leong, Y. A., Hu, X., Ma, C. S., Chevalier, N., Sun, X., Vandenberg, K., Rockman, S., Ding, Y., et al. (2013). Circulating precursor CCR7 (lo) PD-1 (hi) CXCR5 (+) CD4 (+) T cells indicate Tfh cell activity and promote antibody responses upon antigen reexposure. Immunity 39, 770-781.
Howard, L. M., Hoek, K. L., Goll, J. B., Samir, P., Galassie, A., Allos, T. M., Niu, X., Gordy, L. E., Creech, C. B., Prasad, N., et al. (2017). Cell-Based Systems Biology Analysis of Human AS03-Adjuvanted H5N1 Avian Influenza Vaccine Responses: A Phase I Randomized Controlled Trial. PLOS One 12, e0167488.
Hu, X., Yang, D., Zimmerman, M., Liu, F., Yang, J., Kannan, S., Burchert, A., Szulc, Z., Bielawska, A., Ozato, K., et al. (2011). IRF8 regulates acid ceramidase expression to mediate apoptosis and suppresses myelogeneous leukemia. Cancer Res 71, 2882-2891.
Jackson, L. A., Campbell, J. D., Frey, S. E., Edwards, K. M., Keitel, W. A., Kotloff, K. L., Berry, A. A., Graham, I., Atmar, R. L., Creech, C. B., et al. (2015). Effect of Varying Doses of a Monovalent H7N9 Influenza Vaccine With and Without AS03 and MF59 Adjuvants on Immune Response: A Randomized Clinical Trial. JAMA 314, 237-246.
Kalvodova, L. (2010). Squalene-based oil-in-water emulsion adjuvants perturb metabolism of neutral lipids and enhance lipid droplet formation. Biochem Biophys Res Commun 393, 350-355.
Karki, R., Lee, E., Place, D., Samir, P., Mavuluri, J., Sharma, B. R., Balakrishnan, A., Malireddi, R. K. S., Geiger, R., Zhu, Q., et al. (2018). IRF8 Regulates Transcription of Naips for NLRC4 Inflammasome Activation. Cell 173, 920-933 e913.
Karki, R., Lee, E., Sharma, B. R., Banoth, B., and Kanneganti, T. D. (2020). IRF8 Regulates Gram-Negative Bacteria-Mediated NLRP3 Inflammasome Activation and Cell Death. J Immunol 204, 2514-2522.
Kasturi, S. P., Skountzou, I., Albrecht, R. A., Koutsonanos, D., Hua, T., Nakaya, H. I., Ravindran, R., Stewart, S., Alam, M., Kwissa, M., et al. (2011). Programming the magnitude and persistence of antibody responses with innate immunity. Nature 470, 543-547.
Khurana, S., Coyle, E. M., Manischewitz, J., King, L. R., Gao, J., Germain, R. N., Schwartzberg, P. L., Tsang, J. S., Golding, H., and and the, C. H. I. C. (2018). AS03-adjuvanted H5N1 vaccine promotes antibody diversity and affinity maturation, NAI titers, cross-clade H5N1 neutralization, but not H1N1 cross-subtype neutralization. NPJ Vaccines 3, 40.
Kim, E. H., Woodruff, M. C., Grigoryan, L., Maier, B., Lee, S. H., Mandal, P., Cortese, M., Natrajan, M. S., Ravindran, R., Ma, H., et al. (2020). Squalene emulsion-based vaccine adjuvants stimulate CD8 T cell, but not antibody responses, through a RIPK3-dependent pathway. Elife 9.
Krammer, F. (2019). The human antibody response to influenza A virus infection and vaccination. Nat Rev Immunol 19, 383-397.
Kurotaki, D., Nakabayashi, J., Nishiyama, A., Sasaki, H., Kawase, W., Kaneko, N., Ochiai, K., Igarashi, K., Ozato, K., Suzuki, Y., et al. (2018). Transcription Factor IRF8 Governs Enhancer Landscape Dynamics in Mononuclear Phagocyte Progenitors. Cell Rep 22, 2628-2641.
Kurotaki, D., Osato, N., Nishiyama, A., Yamamoto, M., Ban, T., Sato, H., Nakabayashi, J., Umehara, M., Miyake, N., Matsumoto, N., et al. (2013). Essential role of the IRF8-KLF4 transcription factor cascade in murine monocyte differentiation. Blood 121, 1839-1849.
Langley, J. M., Frenette, L., Ferguson, L., Riff, D., Sheldon, E., Risi, G., Johnson, C., Li, P., Kenney, R., Innis, B., et al. (2010). Safety and cross-reactive immunogenicity of candidate AS03-adjuvanted prepandemic H5N1 influenza vaccines: a randomized controlled phase 1/2 trial in adults. J Infect Dis 201, 1644-1653.
Leroux-Roels, I., Roman, F., Forgus, S., Maes, C., De Boever, F., Drame, M., Gillard, P., van der Most, R., Van Mechelen, M., Hanon, E., et al. (2010). Priming with AS03 A-adjuvanted H5N1 influenza vaccine improves the kinetics, magnitude and durability of the immune response after a heterologous booster vaccination: an open non-randomised extension of a double-blind randomised primary study. Vaccine 28, 849-857.
Li, S., Park, Y., Duraisingham, S., Strobel, F. H., Khan, N., Soltow, Q. A., Jones, D. P., and Pulendran, B. (2013). Predicting network activity from high throughput metabolomics. PLOS Comput Biol 9, e1003123.
Locci, M., Havenar-Daughton, C., Landais, E., Wu, J., Kroenke, M. A., Arlehamn, C. L., Su, L. F., Cubas, R., Davis, M. M., Sette, A., et al. (2013). Human circulating PD-1+CXCR3-CXCR5+ memory Tfh cells are highly functional and correlate with broadly neutralizing HIV antibody responses. Immunity 39, 758-769.
Mao, B., Wu, W., Davidson, G., Marhold, J., Li, M., Mechler, B. M., Delius, H., Hoppe, D., Stannek, P., Walter, C., et al. (2002). Kremen proteins are Dickkopf receptors that regulate Wnt/beta-catenin signalling. Nature 417, 664-667. Marazzi, S., Blum, S., Hartmann, R., Gundersen, D., Schreyer, M., Argraves, S., von Fliedner, V., Pytela, R., and Ruegg, C. (1998). Characterization of human fibroleukin, a fibrinogen-like protein secreted by T lymphocytes. J Immunol 161, 138-147.
Matic, I., Sacchi, A., Rinaldi, A., Melino, G., Khosla, C., Falasca, L., and Piacentini, M. (2010). Characterization of transglutaminase type II role in dendritic cell differentiation and function. J Leukoc Biol 88, 181-188.
Mills, E. W., Green, R., and Ingolia, N. T. (2017). Slowed decay of mRNAs enhances platelet specific translation. Blood 129, e38-e48.
Monath, T. P. (2005). Yellow fever vaccine. Expert Rev Vaccines 4, 553-574.
Moris, P., van der Most, R., Leroux-Roels, I., Clement, F., Drame, M., Hanon, E., Leroux-Roels, G. G., and Van Mechelen, M. (2011). H5N1 influenza vaccine formulated with AS03 A induces strong cross-reactive and polyfunctional CD4 T-cell responses. J Clin Immunol 31, 443-454.
Mortensen, M. B., Kjolby, M., Gunnersen, S., Larsen, J. V., Palmfeldt, J., Falk, E., Nykjaer, A., and Bentzon, J. F. (2014). Targeting sortilin in immune cells reduces proinflammatory cytokines and atherosclerosis. J Clin Invest 124, 5317-5322.
Myneni, V. D., Melino, G., and Kaartinen, M. T. (2015). Transglutaminase 2—a novel inhibitor of adipogenesis. Cell Death Dis 6, e1868.
Nakaya, H. I., Hagan, T., Duraisingham, S. S., Lee, E. K., Kwissa, M., Rouphael, N., Frasca, D., Gersten, M., Mehta, A. K., Gaujoux, R., et al. (2015). Systems Analysis of Immunity to Influenza Vaccination across Multiple Years and in Diverse Populations Reveals Shared Molecular Signatures. Immunity 43, 1186-1198.
Nakaya, H. I., Wrammert, J., Lee, E. K., Racioppi, L., Marie-Kunze, S., Haining, W. N., Means, A. R., Kasturi, S. P., Khan, N., Li, G. M., et al. (2011). Systems biology of vaccination for seasonal influenza in humans. Nat Immunol 12, 786-795.
Netea, M. G., Joosten, L. A., Latz, E., Mills, K. H., Natoli, G., Stunnenberg, H. G., O'Neill, L. A., and Xavier, R. J. (2016). Trained immunity: A program of innate immune memory in health and disease. Science 352, aaf1098.
Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nat Methods 12, 453-457.
Newman, A. M., Steen, C. B., Liu, C. L., Gentles, A. J., Chaudhuri, A. A., Scherer, F., Khodadoust, M. S., Esfahani, M. S., Luca, B. A., Steiner, D., et al. (2019). Determining cell type abundance and expression from bulk tissues with digital cytometry. Nat Biotechnol 37, 773-782.
Obermoser, G., Presnell, S., Domico, K., Xu, H., Wang, Y., Anguiano, E., Thompson-Snipes, L., Ranganathan, R., Zeitner, B., Bjork, A., et al. (2013). Systems scale interactive exploration reveals quantitative and qualitative differences in response to influenza and pneumococcal vaccines. Immunity 38, 831-844.
Ogawa, K., Tanaka, K., Ishii, A., Nakamura, Y., Kondo, S., Sugamura, K., Takano, S., Nakamura, M., and Nagata, K. (2001). A novel serum protein that is selectively produced by cytotoxic lymphocytes. J Immunol 166, 6404-6412.
Pan, T., Liu, J., Xu, S., Yu, Q., Wang, H., Sun, H., Wu, J., Zhu, Y., Zhou, J., and Zhu, Y. (2020). ANKRD22, a novel tumor microenvironment-induced mitochondrial protein promotes metabolic reprogramming of colorectal cancer cells. Theranostics 10, 516-536.
Price, M. J., Patterson, D. G., Scharer, C. D., and Boss, J. M. (2018). Progressive Upregulation of Oxidative Metabolism Facilitates Plasmablast Differentiation to a T-Independent Antigen. Cell Rep 23, 3152-3159.
Pulendran, B. (2014). Systems vaccinology: probing humanity's diverse immune systems with vaccines. Proc Natl Acad Sci USA 111, 12300-12306.
Qiu, Y., Yang, S., Pan, T., Yu, L., Liu, J., Zhu, Y., and Wang, H. (2019). ANKRD22 is involved in the progression of prostate cancer. Oncol Lett 18, 4106-4113.
Raab, M., Strebhardt, K., and Rudd, C. E. (2019). Immune adaptor SKAP1 acts a scaffold for Polo-like kinase 1 (PLK1) for the optimal cell cycling of T-cells. Sci Rep 9, 10462.
Risitano, A., Beaulieu, L. M., Vitseva, O., and Freedman, J. E. (2012). Platelets and platelet-like particles mediate intercellular RNA transfer. Blood 119, 6288-6295.
Schmitt, N., Bentebibel, S. E., and Ueno, H. (2014). Phenotype and functions of memory Tfh cells in human blood. Trends Immunol 35, 436-442.
Schwartz, K. L., Kwong, J. C., Deeks, S. L., Campitelli, M. A., Jamieson, F. B., Marchand-Austin, A., Stukel, T. A., Rosella, L., Daneman, N., Bolotin, S., et al. (2016). Effectiveness of pertussis vaccination and duration of immunity. CMAJ 188, E399-E406.
Seifert, U., Bialy, L. P., Ebstein, F., Bech-Otschir, D., Voigt, A., Schroter, F., Prozorovski, T., Lange, N., Steffen, J., Rieger, M., et al. (2010). Immunoproteasomes preserve protein homeostasis upon interferon-induced oxidative stress. Cell 142, 613-624.
Seubert, A., Calabro, S., Santini, L., Galli, B., Genovese, A., Valentini, S., Aprea, S., Colaprico, A., D'Oro, U., Giuliani, M. M., et al. (2011). Adjuvanticity of the oil-in-water emulsion MF59 is independent of Nlrp3 inflammasome but requires the adaptor protein MyD88. Proc Natl Acad Sci USA 108, 11169-11174.
Slifka, M. K., and Ahmed, R. (1998). Long-lived plasma cells: a mechanism for maintaining persistent antibody production. Curr Opin Immunol 10, 252-258.
Slifka, M. K., Antia, R., Whitmire, J. K., and Ahmed, R. (1998). Humoral immunity due to long-lived plasma cells. Immunity 8, 363-372.
Sobolev, O., Binda, E., O'Farrell, S., Lorenc, A., Pradines, J., Huang, Y., Duffner, J., Schulz, R., Cason, J., Zambon, M., et al. (2016). Adjuvanted influenza-H1N1 vaccination reveals lymphoid signatures of age-dependent early responses and of clinical adverse events. Nat Immunol 17, 204-213.
Stoeckius, M., Hafemeister, C., Stephenson, W., Houck-Loomis, B., Chattopadhyay, P. K., Swerdlow, H., Satija, R., and Smibert, P. (2017). Simultaneous epitope and transcriptome measurement in single cells. Nat Methods 14, 865-868.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Suganami, T., Yuan, X., Shimoda, Y., Uchio-Yamada, K., Nakagawa, N., Shirakawa, I., Usami, T., Tsukahara, T., Nakayama, K., Miyamoto, Y., et al. (2009). Activating transcription factor 3 constitutes a negative feedback mechanism that attenuates saturated Fatty acid/toll-like receptor 4 signaling and macrophage activation in obese adipose tissue. Circ Res 105, 25-32.
Ta, M. T., Kapterian, T. S., Fei, W., Du, X., Brown, A. J., Dawes, I. W., and Yang, H. (2012). Accumulation of squalene is associated with the clustering of lipid droplets. FEBS J 279, 4231-4244.
Tatsukawa, H., Furutani, Y., Hitomi, K., and Kojima, S. (2016). Transglutaminase 2 has opposing roles in the regulation of cellular functions as well as cell growth and death. Cell Death Dis 7, e2244.
Thanh Le, T., Andreadakis, Z., Kumar, A., Gomez Roman, R., Tollefsen, S., Saville, M., and Mayhew, S. (2020). The COVID-19 vaccine development landscape. Nat Rev Drug Discov 19, 305-306.
Vono, M., Taccone, M., Caccin, P., Gallotta, M., Donvito, G., Falzoni, S., Palmieri, E., Pallaoro, M., Rappuoli, R., Di Virgilio, F., et al. (2013). The adjuvant MF59 induces ATP release from muscle that potentiates response to vaccination. Proc Natl Acad Sci USA 110, 21095-21100.
Wang, C. Y., Mayo, M. W., Korneluk, R. G., Goeddel, D. V., and Baldwin, A. S., Jr. (1998). NF-kappaB antiapoptosis: induction of TRAF1 and TRAF2 and c-IAP1 and c-IAP2 to suppress caspase-8 activation. Science 281, 1680-1683.
Wanka, C., Brucker, D. P., Bahr, O., Ronellenfitsch, M., Weller, M., Steinbach, J. P., and Rieger, J. (2012). Synthesis of cytochrome C oxidase 2: a p53-dependent metabolic regulator that promotes respiratory function and protects glioma and colon cancer cells from hypoxia-induced cell death. Oncogene 31, 3764-3776.
Waters, L. R., Ahsan, F. M., Wolf, D. M., Shirihai, O., and Teitell, M. A. (2018). Initial B Cell Activation Induces Metabolic Reprogramming and Mitochondrial Remodeling. iScience 5, 99-109.
Winter, O., Moser, K., Mohr, E., Zotos, D., Kaminski, H., Szyska, M., Roth, K., Wong, D. M., Dame, C., Tarlinton, D. M., et al. (2010). Megakaryocytes constitute a functional component of a plasma cell niche in the bone marrow. Blood 116, 1867-1875.
Yang, P., Yu, D., Zhou, J., Zhuang, S., and Jiang, T. (2019). TGM2 interference regulates the angiogenesis and apoptosis of colorectal cancer via Wnt/beta-catenin pathway. Cell Cycle 18, 1122-1134.
Yin, J., Fu, W., Dai, L., Jiang, Z., Liao, H., Chen, W., Pan, L., and Zhao, J. (2017). ANKRD22 promotes progression of non-small cell lung cancer through transcriptional up-regulation of E2F1. Sci Rep 7, 4430.
Zhang, Z., Han, N., and Shen, Y. (2020). S100A12 promotes inflammation and cell apoptosis in sepsis-induced ARDS via activation of NLRP3 in fl ammasome signaling. Mol Immunol 122, 38-48.
Zhao, F., Hoechst, B., Duffy, A., Gamrekelashvili, J., Fioravanti, S., Manns, M. P., Greten, T. F., and Korangy, F. (2012). S100A9 a new marker for monocytic human myeloid-derived suppressor cells. Immunology 136, 176-183.
Zhou, D., Hayashi, T., Jean, M., Kong, W., Fiches, G., Biswas, A., Liu, S., Yosief, H. O., Zhang, X., Bradner, J., et al. (2020). Inhibition of Polo-like kinase 1 (PLK1) facilitates the elimination of HIV-1 viral reservoirs in CD4 (+) T cells ex vivo. Sci Adv 6, eaba1941.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for determining whether a candidate adjuvant provides for a core response substantially similar to a high performing reference adjuvant, the method comprising:
administering a vaccine with the candidate adjuvant, to a mammal;
determining an a core response signature from immune cells; and
predicting whether the candidate adjuvant induces the core response by day 1 changes in expression.

2. The method of claim 1, wherein the core response comprises expression in one or more of TGM2, ANKRD22, and KREMEN1.

3. The method of claim 1, further comprising selecting a candidate adjuvant for clinical use or development, wherein the candidate adjuvant substantially similar to a high performing reference adjuvant.

4. The method of claim 1, wherein the mammal is a mouse, non-human primate, or human.

5. A method is for predicting the durability of an immune response to a candidate vaccine, the method comprising:
administering the candidate vaccine to a mammal;
determining a signature response from a sample comprising immune cells in the mammal, and
predicting durability of response from the signature response.

6. The method of claim 5, wherein the candidate vaccine comprises an adjuvant.

7. The method of claim 5, wherein the vaccine is an mRNA vaccine.

8. The method of claim 5, wherein the vaccine is a viral vector vaccine.

9. The method of claim 5, wherein the vaccine is a live or inactivated virus vaccine.

10. The method of claim 5, wherein the sample comprising immune cells is obtained from 7-10 days following administration of the vaccine; and comparing the signature response to a baseline pre-administration value.

11. The method of claim 5, wherein the sample comprising immune cells is a peripheral blood mononuclear cell sample (PBMCs) comprising platelets.

12. The method of claim 5, wherein the sample comprising immune cells is a platelet rich plasma sample.

13. The method of claim 5, wherein determining a signature response comprises a one-step flow cytometry analysis of platelet RNA content, wherein increased platelet RNA predicts a durable response.

14. The method of claim 5, wherein determining a signature response comprises:
(a) labeling cells present in the sample with reagents that distinguish platelets from other cells in the sample;
(b) labeling cells present in the sample with an RNA selective stain;
(c) analyzing the sample by flow cytometry gated on platelets, to determine RNA content.

15. The method of claim 14, wherein the reagents that distinguish platelets from other cells in the sample comprise an antibody specific for CD41 and an antibody specific for CD61, wherein platelets are CD41⁺CD61⁺.

16. The method of claim 15, wherein the reagents that distinguish platelets from other cells in the sample further comprise one or more of anti-TER119, anti-CD3, anti-CD8, anti-CD19, anti-CD20, anti-CD14, and anti-CD56 antibodies.

17. The method of any of claims 13-16 claim 13, wherein a durable immune response is associated with an increase of at least about 5-fold RNA content relative to baseline.

18. The method of claim 5, wherein the signature response is comprised of expression level data from one or more genes selected from: GPR15, EPS8L1, SLC38A1, GXMM, MGLL, CTTN, XK, PF4, SELP, CDHR5, MYLK, CALD1, CXCL9, SDPR, SPTB, PROS1, PRKAR2B, PPBP, CXCL5, HEMGN, EGF.

19. The method of claim 5, wherein the signature response is determined from a sample obtained about 1 to 7 days or about 10 days following secondary or primary immunization.

20. The method of claim 5, further comprising selecting a candidate adjuvant for clinical use or development, wherein the candidate adjuvant elicits a signature indicative of durable antibody response.

21. (canceled)
